(19) United States
(12) Patent Application Publication
(10) Pub. No.: US 2015/0004178 A1
(43) Pub. Date: Jan. 1, 2015

(54) ANTI-HUMAN SEMA4A ANTIBODIES USEFUL TO TREAT DISEASE

(71) Applicant: BOARD OF REGENTS, THE UNIVERSITY OF TEXAS SYSTEM, Austin, TX (US)

(72) Inventors: Yong-Jun Liu, Pearland, TX (US); Ning Lu, Houston, TX (US); Laura Bover, Pearland, TX (US); Naoya Tsurushita, Palo Alto, CA (US); J. Yun Tso, Menlo Park, CA (US); Shankar Kumar, Pleasanton, CA (US)

(73) Assignee: BOARD OF REGENTS, THE UNIVERSITY OF TEXAS SYSTEM, Austin, TX (US)

(21) Appl. No.: 14/349,836

(22) PCT Filed: Oct. 5, 2012
(86) PCT No.: PCT/US2012/058910

§ 371 Date: Apr. 4, 2014

Related U.S. Application Data
(60) Provisional application No. 61/543,877, filed on Oct. 6, 2011.

(51) Int. Cl. C07K 16/18

(52) U.S. Cl. CPC C07K 16/18; C07K 2317/76
USPC 424/172.1; 530/389.8; 530/387.3

(57) ABSTRACT

Anti-human sema4A antibodies useful in treating autoimmune diseases, cancers and other disease are provided herein. Anti-human sema4A antibodies can inhibit T cell proliferation and Th2 differentiation induced by IL-4, anti-CD3, anti-CD28 and recombinant sema4A.

### CROSS-REFERENCE TO RELATED APPLICATIONS

This application claims the benefit of and the priority in U.S. provisional patent application No. 61/543,877 filed Oct. 6, 2011, incorporated herein by reference.

### STATEMENT REGARDING FEDERALLY SPONSORED RESEARCH OR DEVELOPMENT

This invention was made with government support under R01 AI062888-01 awarded by the National Institute of Health. The government has certain rights in the invention.

### FIELD OF INVENTION

This invention relates generally to modulation of human sema4A, and more particularly to inhibiting human sema4A to block the proliferation and Th2 differentiation induced by anti-CD3, anti-CD-28, IL-4, recombinant sema4A and dendritic cells and treat disease.

### THE NAMES OF PARTIES TO A JOINT RESEARCH AGREEMENT

None.

### REFERENCE TO SEQUENCE LISTING

This disclosure includes a sequence listing submitted as a text file pursuant to 37 C.F.R. §1.52(e)(v) named sequence listing.txt, created on Oct. 5, 2012, with a size of 46,124 bytes, which is incorporated herein by reference. The attached sequence descriptions and Sequence Listing comply with the rules governing nucleotide and/or amino acid sequence disclosures in patent applications as set forth in 37 C.F.R. §§1.821-1.825. The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219 (No. 2):345-373 (1984). The symbols and format used for nucleotide and amino acid sequence data comply with the rules set forth in 37 C.F.R. §1.822.

### BACKGROUND OF THE INVENTION

Semaphorins are a large, conserved family of secreted or membrane-associated ligands that function as either repulsive or attractive cues for growth cones. The semaphorin family is divided into subclasses based on functional domains and sequence similarity. Rice, D., et al., Severe Retinal Degeneration Associated with Disruption of Semaphorin 4A, Investigative Ophthalmology & Visual Science, Vol. 45, No. 8 (2004). Sema4A is a class IV semaphorin. In mice, there is evidence to show that sema4A is expressed by bone-marrow derived and splenic DCs as well as by B cells and activated T cells. Sema4A can stimulate T cell proliferation through its receptor TIM2, which induces IL-2 production and is reportedly up-regulated following B cell stimulation with anti-CD40 monoclonal antibodies. Kumanogoh, A., et al. Nature, 419:629-33, 2002. Sema4A-deficient mice have been shown to exhibit a defective Th1 response. Sema4A seems to be involved in T cell priming and in the regulation of Th1/Th2 responses. Kumanogoh, A. et al. Immunity, 22:305-16, 2005. However, to date, the biological function of sema4A in the human immune system has not been studied.

### BRIEF SUMMARY OF THE INVENTION

Human sema4A protein is highly expressed by antigen presenting cells, such as dendritic cells and activated germinal center B cell, and CD4+Th2 cells. We have discovered that recombinant sema4A protein strongly promotes human CD4+ T cell proliferation and Th2 differentiation induced by IL-4, anti-CD3, anti-CD28 and recombinant sema4A. At the same time, neutralizing mAb can block the biological function. As such, human sema4A (also referred to herein as “hsema4A” or more generally as “sema4A” or “Sema4A”) presents an important and advantageous therapeutic target.

Provided herein are novel monoclonal and humanized antibodies which bind to human sema4A. These antibodies are sometimes referred to herein as “anti-human sema4A antibody” or “anti-human sema4A antibodies,” but may also be referred to as an isolated antibody, a monoclonal antibody, a chimeric antibody, humanized antibody or simply antibody, as presented in the singular or plural.

The anti-human sema4A antibodies taught are useful in the treatment or prevention of acute or chronic diseases and conditions. In one aspect, the isolated antibody, or an antigen-binding portion thereof that binds to human sema4A, is described and is effective as a cancer treatment. The anti-human sema4A antibodies may also be useful in treating human autoimmune disease, allergic disease, inflammatory disease, graft versus host disease and graft rejection.

Further provided are compositions, methods, kits and articles of manufacture based on these antibody binding agents to human sema4A. These anti-human sema4A antibodies can be therapeutic and/or diagnostic agents and are useful in targeting pathological conditions associated with expression and/or activity of human sema4A and its receptors human immunoglobulin-like transcript-4 (hILT-4), human T-cell immunoglobulin domain and human mucin domain 3 (hTIM-3) and human immunoglobulin-like transcript-2 (hILT-2) signaling pathways.

In addition, the isolated antibodies as described herein bind to human sema4A, and may bind to human sema4A encoded from the following genes: NCBI Accession Number HGNC: 10729, Genpept Accession Number 64218, or genes having 90 percent homology thereto. The isolated antibody provided herein may further bind to the human sema4A receptor having one of the following GenBank Accession Numbers: 10288, 84868 and 10859.

As provided herein, exemplary is an isolated antibody which binds to human sema4A comprising: (a) a heavy chain variable region CDR1 comprising the amino acid sequence of SEQ ID NO: 36; (b) a heavy chain variable region CDR2 comprising the amino acid sequence of SEQ ID NO: 37; (c) a heavy chain variable region CDR3 comprising the amino acid sequence of SEQ ID NO. 38; (d) a light chain variable region CDR1 comprising the amino acid sequence of SEQ ID NO. 48; (e) a light chain variable region CDR2 comprising the amino acid sequence of SEQ ID NO. 49; and (f) a light chain variable region CDR3 comprising the amino acid sequence of SEQ ID NO. 50.

Furthermore, another example is an isolated antibody which binds to human sema4A comprising: (a) a heavy chain variable region CDR1 comprising the amino acid sequence of SEQ ID NO: 54; (b) a heavy chain variable region CDR2 comprising the amino acid sequence of SEQ ID NO: 55; (c) a heavy chain variable region CDR3 comprising the amino acid sequence of SEQ ID NO. 56; (d) a light chain variable region CDR1 comprising the amino acid sequence of SEQ ID NO. 66; (e) a light chain variable region CDR2 comprising the amino acid sequence of SEQ ID NO. 67; and (f) a light chain variable region CDR3 comprising the amino acid sequence of SEQ ID NO. 68.

Alternatively, an isolated antibody may have a heavy chain variable region CDR1 comprising the amino acid sequence of SEQ ID NO: 36 or 54; a heavy chain variable region CDR2 comprising the amino acid sequence of SEQ ID NO: 37 or 55; and/or a heavy chain variable region CDR3 comprising the amino acid sequence of SEQ ID NO: 38 or 56, or a heavy chain variable region CDR having 90 percent homology thereto.

Further, an isolated antibody may have a light chain variable region CDR1 comprising the amino acid sequence of SEQ ID NO: 48 or 66; a light chain variable region CDR2 comprising the amino acid sequence of SEQ ID NO: 49 or 67 and/or a light chain variable region CDR3 comprising the amino acid sequence of SEQ ID NO: 50 or 68, or a heavy chain variable region having 90 percent homology thereto.

The isolated antibody may have a light chain variable region (“VL”) comprising the amino acid sequence of SEQ ID NO: 51 or 69, or an amino acid sequence with at least 90 percent identity to the amino acid sequences of SEQ ID NO: 51 or 69. The isolated antibody may have a heavy chain variable region (“VH”) comprising the amino acid sequence of SEQ ID NO: 39, 45, 57 or 63, or an amino acid sequence with at least 90 percent identity to the amino acid sequences of SEQ ID NO: 39, 45, 57 or 63. As such, as an example, the isolated antibody may comprise a variable heavy sequence of SEQ ID NO: 39, 45, 57 or 63 and a variable light sequence of SEQ ID NO: 51, or a sequence having 90 percent homology thereto. Similarly, the isolated antibody can have a variable heavy sequence of SEQ ID NO: 39, 45, 57 or 63 and a variable light sequence of SEQ ID NO: 69 or a sequence having 90 percent homology thereto.

The isolated antibody may have variable light chain encoded by the nucleic acid sequence of SEQ ID NO: 52 or 70, or a nucleic acid sequence with at least 90 percent identity to the nucleotide sequences of SEQ ID NO: 52 or 70. The isolated antibody may have variable heavy chain encoded by a nucleic acid sequence of SEQ ID NO: 40, 46, 58 or 64, or a nucleic acid sequence with at least 90 percent identity to nucleotide sequences of SEQ ID NO: 40, 46, 58 or 64.

Also provided herein are monoclonal antibodies. The monoclonal antibodies may have a variable light chain comprising the amino acid sequence of SEQ ID NO: 73, 75 or 77, or an amino acid sequence with at least 90 percent identity to the amino acid sequences of SEQ ID NO: 73, 75, or 77. Further provided are monoclonal antibodies having a variable heavy chain comprising the amino acid sequence of SEQ ID NO: 72, 74 or 76, or an amino acid sequence with at least 90 percent identity to the amino acid sequences of SEQ ID NO: 72, 74 or 76.

Other antibodies may have any one or more of the following components: a variable light chain; a variable heavy chain; or heavy or light chain variable region CDR1, CDR2, or CDR3, each component comprising an amino acid or a nucleic acid sequence of SEQ ID NO: 1 through 77 as described herein and identified in the sequence listing, or comprising an amino acid or a nucleic acid with at least 90 percent identity to one of the sequences of SEQ ID NO: 1 through 77.

Also provided herein is isolated nucleic acid encoding any of the anti-human sema4A antibodies taught herein. Further provided herein are host cells which comprise the nucleic acid encoding any of the anti-human sema4A antibodies described herein. Methods of producing an antibody (such as the host cell comprising nucleic acid encoding any of the anti-sema4A antibodies described herein) comprising culturing the host cell so that the antibody is produced, and/or recovering the antibody from the host cell, are further provided.

### BRIEF DESCRIPTION OF THE DRAWINGS

So that the manner in which the above-recited features, aspects and advantages of the invention, as well as others that will become apparent, are attained and can be understood in detail, more particular description of the invention briefly summarized above can be had by reference to the embodiments thereof that are illustrated in the drawings that form a part of this specification. It is to be noted, however, that the appended drawings illustrate some embodiments of the invention and are, therefore, not to be considered limiting of the invention's scope, for the invention can admit to other equally effective embodiments.

FIGS. 1A, 1A1, 1B and 1C shows sema4A expression on human mDCs

FIGS. 2A and 2B provides immunohistology staining of sema4A in human tonsil.

FIGS. 3A and 3B depict anti-human sema4A mAb blocks interaction between mDC and T cells.

FIGS. 4A, 4B, 4C, and 4D show that sema4A enhances CD4+ naïve T cells proliferation.

FIGS. 5A, 5B, 5C, 5D, and 5E show anti-human sema4A mAbs block sema4A mediated CD4+ T cell proliferation under TCR triggering.

FIGS. 6A, 6B, 6C and 6D show sema4A regulates Th2 cytokine secretion on Th2 primed CD4+ Th.

FIGS. 7A, 7B, 7C and 7D show sema4A up-regulates CRTH2+ memory Th2 cells producing Th2 cytokines and down regulates Th1 cytokine under both T cell activation and Th2 culture condition.

FIGS. 8A, 8B, 8C, 8D, 8E, 8F, 8G, 8H, and 81 show anti-sema4A monoclonal antibody suppresses Th2 cytokines (IL-4, IL-5 and IL-13) production induced by sema4A in CD4+ Th2 cells.

FIG. 9 shows the identification of anti-human sema4A monoclonal antibodies from the first batch.

FIGS. 10A, 10B, 10C, 10D, and 10E show the results from the functional assay of the anti-human sema4A monoclonal antibody clone numbers 126-65, 126-28 and 126-63.

FIGS. 11A, 11B, 11C, and 11D show the results from the functional assay of the anti-human sema4A monoclonal antibody clone numbers 126-25 and 126-31.

FIG. 12 shows the identification of anti-human sema4A monoclonal antibodies from the second batch.

FIG. 13 shows the results from the functional assay of anti-human sema4A monoclonal antibody clone numbers 161-70-1, 161-90, 161-96B, 161-110B, 161-117, 161-118A and 161-66.

FIG. 14 shows the results from the functional assay of anti-human sema4A monoclonal antibody clone numbers 161-15B, 161-18B, 161-33, 161-35, 161-44, 161-49 and 161-51.

FIG. 15 shows the results of an analysis of LB70 antibodies binding to Lisema4A.

FIGS. 16A and 16B show the results of an analysis of LB51 antibodies binding to L/sema4A.

FIGS. 17A and 17B show that positive clones of anti-human Sema4A mAbs detect the Rhesus monkey Sema4A expression on PBMC.

FIGS. 18A, 18B, 18C, and 18D show the results of the analysis of two strains of humanized mAbs against hSema4A.

FIGS. 19A and 19B is show sema4A receptors cloning with human T cell expressing cDNA library screening.

FIGS. 20A and 20B show that sema4A receptors bind to sema4A transfected cell line.

FIGS. 21A and 21B show receptor-Fc fusion protein blocks human CD4+ T cells proliferation mediated by rhSema4A.

FIGS. 22A, 22B, and 22C show sema4A is specifically over-expressed in human Asthma lung tissue.

FIG. 23 shows the nucleotide sequence of mouse 161-51-1 VH cDNA along with the deduced amino acid sequence. This figure also provides amino acid residues shown in single letter code. The signal peptide sequence is in italic. The N-terminal amino acid residue (Q) of the mature VH is double-underlined and bold. CDR sequences according to the definition of Kabat et al. (1991) are underlined.

FIG. 24 shows the nucleotide sequence of mouse 161-51-1 VL cDNA along with the deduced amino acid sequence. This figure also provides amino acid residues shown in single letter code. The signal peptide sequence is in italic. The N-terminal amino acid residue (E) of the mature VL is double-underlined and bold. CDR sequences according to the definition of Kabat et al. (1991) are underlined.

FIG. 25 shows the nucleotide sequence of mouse 161-70-1 VH cDNA along with the deduced amino acid sequence. This figure also provides amino acid residues shown in single letter code. The signal peptide sequence is in italic. The N-terminal amino acid residue (E) of the mature VH is double-underlined and bold. CDR sequences according to the definition of Kabat et al. (1991) are underlined.

FIG. 26 shows the nucleotide sequence of mouse 161-70-1 VL cDNA along with the deduced amino acid sequence. Amino acid residues are shown in single letter code. The signal peptide sequence is in italic. The N-terminal amino acid residue (D) of the mature VL is double-underlined and bold. CDR sequences according to the definition of Kabat et al. (1991) are underlined.

FIG. 27 shows the nucleotide sequence of mouse 161-15B-1 VH cDNA along with the deduced amino acid sequence. Amino acid residues are shown in single letter code. The signal peptide sequence is in italic. The N-terminal amino acid residue (Q) of the mature VH is double-underlined and bold. CDR sequences according to the definition of Kabat et al. (1991) are underlined.

FIG. 28 shows the nucleotide sequence of mouse 161-15B-1 VL1 cDNA along with the deduced amino acid sequence. Amino acid residues are shown in single letter code. The signal peptide sequence is in italic. The N-terminal amino acid residue (N) of the mature VL is double-underlined and bold. CDR sequences according to the definition of Kabat et al. (1991) are underlined.

FIG. 29 shows the nucleotide sequence of mouse 161-15B-1 VL2 cDNA along with the deduced amino acid sequence. Amino acid residues are shown in single letter code. The signal peptide sequence is in italic. The N-terminal amino acid residue (A) of the mature VL is double-underlined and bold. CDR sequences according to the definition of Kabat et al. (1991) are underlined.

FIG. 30 provides the nucleotide and amino acid sequences of a variable heavy region of the anti-human sema4A humanized antibody, HuLB51. The HuLB51 VH1 gene is flanked by SpeI and HindIII sites (underlined) and shown along with the deduced amino acid sequence. Amino acid residues are shown in single letter code. The signal peptide sequence is in italic. The N-terminal amino acid residue (Q) of the mature VH is double-underlined. CDR sequences according to the definition of Kabat et al. (Sequences of Proteins of Immunological Interests, Fifth edition, NIH Publication No. 91-3242, U.S. Department of Health and Human Services, 1991) are underlined. The intron sequence is shown in italics.

FIG. 31 provides the nucleotide and amino acid sequences of a variable heavy region of the anti-human sema4A humanized antibody, HuLB51. The nucleotide sequence of the HuLB51 VH2 gene flanked by SpeI and HindIII sites (underlined) is shown along with the deduced amino acid sequence. Amino acid residues are shown in single letter code. The signal peptide sequence is in italic. The N-terminal amino acid residue (Q) of the mature VH is double-underlined. CDR sequences according to the definition of Kabat et al. (1991) are underlined. The intron sequence is in italic.

FIG. 32 provides the nucleotide and amino acid sequences of a variable light region of the anti-human sema4A humanized antibody, HuLB51. The nucleotide sequence of the HuLB51 VL gene flanked by NheI and EcoRI sites (underlined) is shown along with the deduced amino acid sequence. Amino acid residues are shown in single letter code. The signal peptide sequence is in italic. The N-terminal amino acid residue (D) of the mature VL is double-underlined. CDR sequences according to the definition of Kabat et al. (1991) are underlined. The intron sequence is in italic.

FIG. 33 provides the nucleotide and amino acid sequences of a variable heavy region of the anti-human sema4A humanized antibody, HuLB70 VH1. Nucleotide sequence of the HuLB70 VH 1 gene flanked by SpeI and HindIII sites (underlined) is shown along with the deduced amino acid sequence. Amino acid residues are shown in single letter code. The signal peptide sequence is in italic. The N-terminal amino acid residue (Q) of the mature VH is double-underlined. CDR sequences according to the definition of Kabat et al. (1991) are underlined. The intron sequence is in italic.

FIG. 34 provides the nucleotide and amino acid sequences of a variable heavy region of the anti-human sema4A humanized antibody, HuLB70 VH2. Nucleotide sequence of the HuLB70 VH2 gene flanked by SpeI and HindIII sites (underlined) is shown along with the deduced amino acid sequence. Amino acid residues are shown in single letter code. The signal peptide sequence is in italic. The N-terminal amino acid residue (Q) of the mature VH is double-underlined. CDR sequences according to the definition of Kabat et al. (1991) are underlined. The intron sequence is in italic.

FIG. 35 provides the nucleotide and amino acid sequences of a variable light region of the anti-human sema4A humanized antibody, HuLB70 VL. Nucleotide sequence of the HuLB70 VL gene flanked by NheI and EcoRI sites (underlined) is shown along with the deduced amino acid sequence. Amino acid residues are shown in single letter code. The signal peptide sequence is in italic. The N-terminal amino acid residue (D) of the mature VL is double-underlined. CDR sequences according to the definition of Kabat et al. (1991) are underlined. The intron sequence is in italic.

### DETAILED DESCRIPTION OF THE INVENTION

Monoclonal, chimeric and humanized antibodies (sometimes referred to herein as an “anti-human sema4A antibody” and/or other variations of the same) that bind human sema4A receptor are provided herein. These antibodies are useful in the treatment or prevention of acute or chronic diseases or conditions whose pathology involves human sema4A. In one aspect, an isolated human antibody, or an antigen-binding portion thereof, that binds to human sema4A and is effective as a cancer treatment or treatment against an autoimmune disease is described. Any of the anti-human sema4A antibodies disclosed herein may be used as a medicament. Any one or more of the anti-human sema4A antibodies may be used to treat one or more a variety of cancers or autoimmune disease described herein.

Anti-human sema4 antibodies provided herein block CD4+ T cell proliferation and Th2 differentiation. As described below, the anti-human sema4A antibody can be a monoclonal or polyclonal antibody that binds to human sema4A. The origin of the monoclonal and/or polyclonal antibody can be rabbit, rat and. mouse. Further, the anti-human sema4A antibody can be a chimeric antibody, an affinity matured antibody, a humanized antibody, or a human antibody. Moreover, the anti-human sema4A antibody can be an antibody fragment, or a Fab, Fab′, Fab′-SH, F(ab′)₂, or scFv.

As presented herein, the chimeric anti-human sema4 antibody comprises antigen binding sequences from a non-human donor grafted to a heterologous non-human, human or humanized sequence (e.g., framework and/or constant domain sequences). The non-human donor could be a mouse, rat or rabbit. Also, the antigen binding sequence could be synthetic, e.g. obtained by mutagenesis (e.g., phage display screening, etc.). A chimeric antibody has murine V regions and human C region. The murine light chain V region may be fused to a human kappa light chain.

The murine heavy chain V region may be fused to a human IgG1 C region.

The isolated antibodies as described herein bind to human sema4A, and may bind to human sema4A encoded from the following genes: NCBI Accession Number HGNC:10729, Genpept Accession Number 64218, or genes having 90 percent homology thereto. The isolated antibody provided herein may further bind to the human sema4A receptor having one of the following GenBank Accession Numbers: 10288, 84868 and 10859.

As taught herein, exemplary is an isolated antibody which binds to human sema4A comprising: (a) a heavy chain variable region CDR1 comprising the amino acid sequence of SEQ ID NO: 36; (b) a heavy chain variable region CDR2 comprising the amino acid sequence of SEQ ID NO: 37; (c) a heavy chain variable region CDR3 comprising the amino acid sequence of SEQ ID NO. 38; (d) a light chain variable region CDR1 comprising the amino acid sequence of SEQ ID NO. 48; (e) a light chain variable region CDR2 comprising the amino acid sequence of SEQ ID NO. 49; and (f) a light chain variable region CDR3 comprising the amino acid sequence of SEQ ID NO. 50.

Furthermore, another example is an isolated antibody which binds to human sema4A comprising: (a) a heavy chain variable region CDR1 comprising the amino acid sequence of SEQ ID NO: 54; (b) a heavy chain variable region CDR2 comprising the amino acid sequence of SEQ ID NO: 55; (c) a heavy chain variable region CDR3 comprising the amino acid sequence of SEQ ID NO. 56; (d) a light chain variable region CDR1 comprising the amino acid sequence of SEQ ID NO. 66; (e) a light chain variable region CDR2 comprising the amino acid sequence of SEQ ID NO. 67; and (f) a light chain variable region CDR3 comprising the amino acid sequence of SEQ ID NO. 68.

Alternatively, an isolated antibody may have a heavy chain variable region CDR1 comprising the amino acid sequence of SEQ ID NO: 36 or 54; a heavy chain variable region CDR2 comprising the amino acid sequence of SEQ ID NO: 37 or 55; and/or a heavy chain variable region CDR3 comprising the amino acid sequence of SEQ ID NO: 38 or 56, or a heavy chain variable region CDR having 90 percent homology thereto.

Further, an isolated antibody may have a light chain variable region CDR1 comprising the amino acid sequence of SEQ ID NO: 48 or 66; a light chain variable region CDR2 comprising the amino acid sequence of SEQ ID NO: 49 or 67 and/or a light chain variable region CDR3 comprising the amino acid sequence of SEQ ID NO: 50 or 68, or a heavy chain variable region having 90 percent homology thereto.

The isolated antibody may have a light chain variable region (“VL”) comprising the amino acid sequence of SEQ ID NO: 51 or 69, or an amino acid sequence with at least 90 percent identity to the amino acid sequences of SEQ ID NO: 51 or 69. The isolated antibody may have a heavy chain variable region (“VH”) comprising the amino acid sequence of SEQ ID NO: 39, 45, 57 or 63, or an amino acid sequence with at least 90 percent identity to the amino acid sequences of SEQ ID NO: 39, 45, 57 or 63. As such, as an example, the isolated antibody may comprise a variable heavy sequence of SEQ ID NO: 39, 45, 57 or 63 and a variable light sequence of SEQ ID NO: 51, or a sequence having 90 percent homology thereto. Similarly, the isolated antibody can have a variable heavy sequence of SEQ ID NO: 39, 45, 57 or 63 and a variable light sequence of SEQ ID NO: 69 or a sequence having 90 percent homology thereto.

The isolated antibody may have variable light chain encoded by the nucleic acid sequence of SEQ ID NO: 52 or 70, or a nucleic acid sequence with at least 90 percent identity to the nucleotide sequences of SEQ ID NO: 52 or 70. The isolated antibody may have variable heavy chain encoded by a nucleic acid sequence of SEQ ID NO: 40, 46, 58 or 64, or a nucleic acid sequence with at least 90 percent identity to nucleotide sequences of SEQ ID NO: 40, 46, 58 or 64.

Also provided herein are monoclonal and chimeric antibodies. These antibodies may have a variable light chain comprising the amino acid sequence of SEQ ID NO: 73, 75 or 77, or an amino acid sequence with at least 90 percent identity to the amino acid sequences of SEQ ID NO: 73, 75, or 77. Further provided are monoclonal antibodies having a variable heavy chain comprising the amino acid sequence of SEQ ID NO: 72, 74 or 76, or an amino acid sequence with at least 90 percent identity to the amino acid sequences of SEQ ID NO: 72, 74 or 76.

Other antibodies may have any one or more of the following components: a variable light chain; a variable heavy chain; or heavy or light chain variable region CDR1, CDR2, or CDR3, each component comprising an amino acid or a nucleic acid sequence of SEQ ID NO: 1 through 77 as described herein and identified in the sequence listing, or comprising an amino acid or a nucleic acid with at least 90 percent identity to one of the sequences of SEQ ID NO: 1 through 77.

As taught herein, exemplary is yet another isolated antibody which binds to human sema4A comprising: (a) a heavy chain variable region CDR1 comprising the amino acid sequence of SEQ ID NO: 1; (b) a heavy chain variable region CDR2 comprising the amino acid sequence of SEQ ID NO: 2; (c) a heavy chain variable region CDR3 comprising the amino acid sequence of SEQ ID NO: 3; (d) a light chain variable region CDR1 comprising the amino acid sequence of SEQ ID NO: 6; (e) a light chain variable region CDR2 comprising the amino acid sequence of SEQ ID NO: 7; and (f) a light chain variable region CDR3 comprising the amino acid sequence of SEQ ID NO: 8.

Furthermore, another example is an isolated antibody which binds to human sema4A comprising: (a) a heavy chain variable region CDR1 comprising the amino acid sequence of SEQ ID NO: 11; (b) a heavy chain variable region CDR2 comprising the amino acid sequence of SEQ ID NO: 12; (c) a heavy chain variable region CDR3 comprising the amino acid sequence of SEQ ID NO. 13; (d) a light chain variable region CDR1 comprising the amino acid sequence of SEQ ID NO. 16; (e) a light chain variable region CDR2 comprising the amino acid sequence of SEQ ID NO. 17; and (f) a light chain variable region CDR3 comprising the amino acid sequence of SEQ ID NO. 18.

Furthermore, another example is an isolated antibody which binds to human sema4A comprising: (a) a heavy chain variable region CDR1 comprising the amino acid sequence of SEQ ID NO: 21; (b) a heavy chain variable region CDR2 comprising the amino acid sequence of SEQ ID NO: 22; (c) a heavy chain variable region CDR3 comprising the amino acid sequence of SEQ ID NO. 23; (d) a light chain variable region CDR1 comprising the amino acid sequence of SEQ ID NO. 26; (e) a light chain variable region CDR2 comprising the amino acid sequence of SEQ ID NO. 27; and (f) a light chain variable region CDR3 comprising the amino acid sequence of SEQ ID NO. 28.

Furthermore, another example is an isolated antibody which binds to human sema4A comprising: (a) a heavy chain variable region CDR1 comprising the amino acid sequence of SEQ ID NO: 21; (b) a heavy chain variable region CDR2 comprising the amino acid sequence of SEQ ID NO: 22; (c) a heavy chain variable region CDR3 comprising the amino acid sequence of SEQ ID NO. 23; (d) a light chain variable region CDR1 comprising the amino acid sequence of SEQ ID NO. 26; (e) a light chain variable region CDR2 comprising the amino acid sequence of SEQ ID NO. 27; and (f) a light chain variable region CDR3 comprising the amino acid sequence of SEQ ID NO. 28.

Furthermore, another example is an isolated antibody which binds to human sema4A comprising: (a) a heavy chain variable region CDR1 comprising the amino acid sequence of SEQ ID NO: 21; (b) a heavy chain variable region CDR2 comprising the amino acid sequence of SEQ ID NO: 22; (c) a heavy chain variable region CDR3 comprising the amino acid sequence of SEQ ID NO. 23; (d) a light chain variable region CDR1 comprising the amino acid sequence of SEQ ID NO. 31; (e) a light chain variable region CDR2 comprising the amino acid sequence of SEQ ID NO. 32; and (f) a light chain variable region CDR3 comprising the amino acid sequence of SEQ ID NO. 33.

Alternatively, an isolated antibody may have a heavy chain variable region CDR1 comprising the amino acid sequence of SEQ ID NO: 1, 11 or 21; a heavy chain variable region CDR2 comprising the amino acid sequence of SEQ ID NO: 2, 12 or 22; and/or a heavy chain variable region CDR3 comprising the amino acid sequence of SEQ ID NO: 3, 13 or 23, or a heavy chain variable region CDR having 90 percent homology thereto.

Further, an isolated antibody may have a light chain variable region CDR1 comprising the amino acid sequence of SEQ ID NO: 6, 16, 26, or 31; a light chain variable region CDR2 comprising the amino acid sequence of SEQ ID NO: 7, 17, 27, or 32 and/or a light chain variable region CDR3 comprising the amino acid sequence of SEQ ID NO: 8, 18, 28, or 33, or a heavy chain variable region having 90 percent homology thereto.

Also provided herein is isolated nucleic acid encoding any of the anti-human sema4A antibodies taught herein. Further provided herein are host cells which comprise the nucleic acid encoding any of the anti-human sema4A antibodies described herein. Methods of producing an antibody (such as the host cell comprising nucleic acid encoding any of the anti-sema4A antibodies described herein) comprising culturing the host cell so that the antibody is produced, and/or recovering the antibody from the host cell, are further provided.

Humanized antibodies can be generated from the monoclonal antibody sequences and include those antibodies that have amino acid substitutions in the FR and affinity maturation variants with changes in the grafted CDRs. The substituted amino acids in the CDR or FR are not limited to those present in the donor or recipient antibody. The antibodies provided herein may further comprise changes in amino acid residues in the Fc region that lead to improved effector function including enhanced CDC and/or ADCC function and B-cell killing. Other antibodies may include those having specific changes that improve stability.

### DEFINITIONS

The term “antibody” includes an immunoglobulin molecule comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

Generally, the terms “cancer” and “cancerous” refer to or describe the physiological condition in mammals that is typically characterized by unregulated cell growth. More specifically, cancers which can be treated or prevented using any one or more of the antibodies described herein or a variant thereof, include, but are not limited to, carcinoma, lymphoma, blastoma, sarcoma, and leukemia. More particular examples of such cancers include, but are not limited to, squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer and gastrointestinal stromal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, melanoma, superficial spreading melanoma, lentigo maligna melanoma, acral lentiginous melanomas, nodular melanomas, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. The antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. However, the isolated antibody can be prepared by at least one purification step.

An “isolated” nucleic acid molecule is a nucleic acid molecule that is identified and separated from at least one contaminant nucleic acid molecule with which it is ordinarily associated in the natural source of the antibody nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the antibody where, for example, the nucleic acid molecule is in a chromosomal location different from that of natural cells.

The terms “Kabat numbering”, “Kabat definitions” and “Kabat labeling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242)

The term “variable domain residue numbering as in Kabat” or “amino acid position numbering as in Kabat”, and variations thereof, refers to the numbering system used for heavy chain variable domains or light chain variable domains of the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence may contain fewer or additional amino acids corresponding to a shortening of, or insertion into, a FR or CDR of the variable domain. The Kabat numbering of residues may be determined for a given antibody by alignment at regions of homology of the sequence of the antibody with a “standard” Kabat numbered sequence.

“Binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention.

The term “vector,” as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “recombinant vectors”). In general, expression vectors of utility in recombinant DNA techniques are often in the form of plasmids. In the present specification, “plasmid” and “vector” may be used interchangeably as the plasmid is the most commonly used form of vector.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Not all linkages in a polynucleotide need be identical. The preceding description applies to all polynucleotides referred to herein, including RNA and DNA.

“Oligonucleotide,” as used herein, generally refers to short, generally single stranded, generally synthetic polynucleotides that are generally, but not necessarily, less than about 200 nucleotides in length. The terms “oligonucleotide” and “polynucleotide” are not mutually exclusive. The description above for polynucleotides is equally and fully applicable to oligonucleotides.

The terms “antibody” and “immunoglobulin” are used interchangeably in the broadest sense and include monoclonal antibodies (for e.g., full length or intact monoclonal antibodies), polyclonal antibodies, multivalent antibodies, multispecific antibodies (e.g., bispecific antibodies so long as they exhibit the desired biological activity) and may also include certain antibody fragments. An antibody can be human, humanized and/or affinity matured.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a .beta.-sheet configuration, connected by three CDRs, which form loops connecting, and in some cases forming part of, the .beta.-sheet structure. The CDRs in each chain are held together in close proximity by the FR regions and, with the CDRs from the other chain, contribute to the formation of the antigen-binding site of antibodies (see Kabat et al., Sequences of Proteins of Immunological Interest, Fifth Edition, National Institute of Health. Bethesda, Md. (1991)). The constant domains are not involved directly in binding an antibody to an antigen, but exhibit various effector functions, such as participation of the antibody in antibody-dependent cellular toxicity.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Fv” is the minimum antibody fragment which contains a complete antigen-recognition and -binding site. In a two-chain Fv species, this region consists of a dimer of one heavy- and one light-chain variable domain in tight, non-covalent association. In a single-chain Fv species, one heavy- and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa (.kappa.) and lambda (.lamda.), based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG, and IgM, and several of these can be further divided into subclasses (isotypes), e.g., IgG₁, IgG₂, IgG₃, IgG₄, IgA₁, and IgA₂. The heavy-chain constant domains that correspond to the different classes of immunoglobulins are called .alpha., .delta., .epsilon., .gamma., and .mu., respectively. The subunit structures and three-dimensional configurations of different classes of immunoglobulins are well known.

“Antibody fragments” comprise only a portion of an intact antibody, wherein the portion preferably retains at least one, preferably most or all, of the functions normally associated with that portion when present in an intact antibody. Examples of antibody fragments include Fab, Fab′, F(ab′)₂, and Fv fragments; diabodies; linear antibodies; single-chain antibody molecules; and multispecific antibodies formed from antibody fragments. In one embodiment, an antibody fragment comprises an antigen binding site of the intact antibody and thus retains the ability to bind antigen. In another embodiment, an antibody fragment, for example one that comprises the Fc region, retains at least one of the biological functions normally associated with the Fc region when present in an intact antibody, such as FcRn binding, antibody half life modulation, ADCC function and complement binding. In one embodiment, an antibody fragment is a monovalent antibody that has an in vivo half life substantially similar to an intact antibody. For e.g., such an antibody fragment may comprise on antigen binding arm linked to an Fc sequence capable of conferring in vivo stability to the fragment.

The term “hypervariable region”, “HVR”, or “HV”, when used herein refers to the regions of an antibody variable domain which are hypervariable in sequence and/or form structurally defined loops. Generally, antibodies comprise six hypervariable regions; three in the VH (H1, H2, H3), and three in the VL (L1, L2, L3). A number of hypervariable region delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM hypervariable regions represent a compromise between the Kabat CDRs and Chothia structural loops, and are used by Oxford Molecular's AbM antibody modeling software. The “contact” hypervariable regions are based on an analysis of the available complex crystal structures.

Hypervariable regions may comprise “extended hypervariable regions” as follows: 24-36 (L1), 46-56 (L2) and 89-97 (L3) in the VL and 26-35 (H1), 47-66 or 49-66 or 50 to 66 (H2) and 93-101 or 93-102 (H3) in the VH. The variable domain residues are numbered according to Kabat et al., supra for each of these definitions.

“Framework” or “FR” residues are those variable domain residues other than the hypervariable region residues as herein defined.

“Humanized” forms of non-human (e.g., murine) antibodies are chimeric antibodies that contain minimal sequence derived from non-human immunoglobulin. For the most part, humanized antibodies are human immunoglobulins in which residues from a hypervariable region of the recipient are replaced by residues from a hypervariable region of a non-human species such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all or at least one, and typically two, variable domains, in which all or substantially all of the hypervariable loops correspond to those of a non-human immunoglobulin and all, or substantially all, FRs are those of a human immunoglobulin sequence. The humanized antibody optionally will also comprise at least a portion of an immunoglobulin constant region (Fc), typically that of a human immunoglobulin.

“Chimeric” antibodies (immunoglobulins) have the total or portion of the heavy and/or light chain identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. Humanized antibody as used herein is a subset of chimeric antibodies.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding.

An “antigen” is a predetermined antigen to which an antibody can selectively bind. The target antigen may be polypeptide, carbohydrate, nucleic acid, lipid, hapten or other naturally occurring or synthetic compound.

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues.

An “affinity matured” antibody is one with one or more alterations in one or more CDRs thereof which result in an improvement in the affinity of the antibody for antigen, compared to a parent antibody which does not possess those alteration(s).

A “blocking” antibody or an “antagonist” antibody is one which inhibits or reduces biological activity of the antigen it binds. Preferred blocking antibodies or antagonist antibodies substantially or completely inhibit the biological activity of the antigen. In a similar manner, the term “agonist” is used herein to include any molecule which promotes, enhances or stimulates the biological activity of the antigen it binds.

A “disorder” or “disease” is any condition that would benefit from treatment with a substance/molecule or method of the invention. This includes chronic and acute disorders or diseases including those pathological conditions which predispose the mammal to the disorder in question. Non-limiting examples of disorders linked to T cell activation and proliferation and to be treated include human autoimmune and inflammatory diseases as well as graft versus host diseases and graft rejection. These diseases include but are not limited to rheumatoid arthritis, lupus erythematosus, autoimmune diabetes, transplantations, multiple sclerosis, osteoarthritis, Crohn's disease, ulcerative colitis, and auto-immune diseases such as lupus and mixed auto-immune disease, insulin-dependent diabetes mellitus (IDDM), diabetes mellitus, experimental autoimmune encephalomyelitis, acute disseminated encephalomyelitis, arthritis, rheumatoid arthritis, experimental autoimmune arthritis, myasthenia gravis, thyroiditis, Hashimoto's disease, primary myxedema, thyrotoxicosis, pernicious anemia, autoimmune atrophic gastritis, Addison's disease, premature menopause, male infertility, juvenile diabetes, Goodpasture's syndrome, pemphigus vulgaris, pemphigoid, sympathetic ophthalmia, phacogenic uveitis, autoimmune haemolyticanaemia, idiopathic leucophenia, primary biliary cirrhosis, active chronic hepatitis Hbₛ₋ᵥₑ, cryptogenic cirrhosis, ulcerative colitis, Sjogren's syndrome, scleroderma, Wegener's granulomatosis, Poly/Dermatomyositis, discoid LE, psoriasis, Ankylosingspondylitisis, Antiphospholipid antibody syndrome, Aplastic anemia, Autoimmune hepatitis, Cocliac disease, Graves' disease, Guillain-Barre syndrome (GBS), Idiopathic thrombocytopenic purpura, Opsoclonus myoclonus syndrome (OMS), Optic neuritis, ORd's thyroiditis, Pemphigus, Polyarthritis, Primary biliary cirrhosis, Reiter's syndrome, Takayasu's, Temporal arteritis, Warm autoimmune hemolytic anemia, Wegener's granulomatosis, Alopecia universalis, Behcet's disease, Chagas' disease, Chronic fatigue syndrome, Dysautonomia, Endometriosis, Hidradenitis suppurativa, Interstitial cystitis, Neuromyotonia, Sarcoidosis, Scleroderma, Ulcerative colitis, Vitiligo, Vulvodynia, inflammatory skin diseases, allergic contact dermatitis, H. pylory gastritis, chronic nasal inflammatory disease, arteriosclerosis and graft versus host disease.

An “autoimmune disease” herein is a disease or disorder arising from and directed against an individual's own tissues or organs or a co-segregate or manifestation thereof or resulting condition therefrom. In many of these autoimmune and inflammatory disorders, a number of clinical and laboratory markers may exist, including, but not limited to, hypergammaglobulinemia, high levels of autoantibodies, antigen-antibody complex deposits in tissues, benefit from corticosteroid or immunosuppressive treatments, and lymphoid cell aggregates in affected tissues.

As used herein, “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, decreasing the rate of disease progression, amelioration or palliation of the disease state, and remission or improved prognosis.

An “individual” is a vertebrate such as a mammal or a human. Mammals include, but are not limited to, farm animals (such as cows), sport animals, pets (such as cats, dogs and horses), primates, mice and rats.

An “effective amount” refers to an amount effective, at dosages and for periods of time necessary, to achieve the desired therapeutic or prophylactic result.

A “therapeutically effective amount” of a substance/molecule of the invention, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

As noted above, the biological function of sema4A in the human immune system has not been studied. In a previous study, we showed that CRTH2+ memory Th2 cells play an important role in maintaining Th2 responses that relate to allergic disease. Specifically, we found that CRTH2+ memory Th2 cells can rapidly produce high levels of Th2 cytokines Il-4, IL-5, and IL-13 immediately upon activation. Furthermore, we found that TSLP-activated DCs induce robust expansion and further Th2 progression of CD4+ Th2 memory cells (Wang, Y H, et al. Immunity. 24(6):827-38, 2006). Through extensive microarray expression analyses, we found that human CRTH2+ Th2 memory cells selectively express two surface receptors including IL-25 receptor and Sema4A. (Want, Y H et al. J Exp Med. 204(8):1837-47, 2007).

For the first time, we demonstrate that Sema4A has a unique function in co-stimulating T cell proliferation and regulating the Th2 response in humans. To explore the mechanism of Sema4A, we generated several batches of monoclonal antibodies against human Sema4A and performed a functional assay of the antibodies. Then, we obtained 16 total clones of monoclonal antibodies which can block recombinant human Sema4A-mediated T cell proliferation and Th2 differentiation.

### Compositions of the Invention and Methods of Making Same

This invention encompasses compositions, including pharmaceutical compositions, comprising an anti-human sema4a antibody, and polynucleotides comprising sequences encoding an anti-human sema4a antibody. As used herein, compositions comprise one or more antibodies that bind to human sema4a, and/or one or more polynucleotides comprising sequences encoding one or more antibodies that bind to human sema4A. These compositions may further comprise suitable carriers, such as pharmaceutically acceptable excipients including buffers, which are well known in the art.

The anti-human sema4A antibodies are monoclonal. Also encompassed within the scope of the invention are Fab, Fab′, Fab′-SH and F(ab′)₂ fragments of the anti-human sema4A antibodies provided herein. These antibody fragments can be created by traditional means, such as enzymatic digestion, or may be generated by recombinant techniques. Such antibody fragments may be chimeric or humanized. These fragments are useful for the diagnostic and therapeutic purposes set forth below.

Monoclonal antibodies are obtained from a population of substantially homogeneous antibodies, i.e., the individual antibodies comprising the population are identical except for possible naturally occurring mutations that may be present in minor amounts.

The anti-sema4A antibodies presented herein may be made by using combinatorial libraries to screen for synthetic antibody clones with the desired activity or activities. In principle, synthetic antibody clones are selected by screening phage libraries containing phage that display various fragments of antibody variable region (Fv) fused to phage coat protein. Clones expressing Fv fragments capable of binding to the desired antigen are adsorbed to the antigen and thus separated from the non-binding clones in the library. The binding clones are then eluted from the antigen, and can be further enriched by additional cycles of antigen adsorption/elution.

### Anti-Human Sema4A Monoclonal Antibodies

Generation of anti-human sema4A monoclonal antibodies can be performed, for example, by immunizing mice with a mouse cell line transfected with human-sema4A following established protocols.

We design an exhaustive screening to detect those clones that trigger sema4A signaling (i.e., agonists antibodies) by inhibiting the generation and function of Tr1 cells. Those clones were further purified. Agonist antibodies against sema4A may be humanized and use in clinical protocols for human anti tumor therapy, either alone or in combination with anti tumor vaccination and other adjuvants. Several different tumor types could be the target of these antibodies, including melanoma, lymphoma and breast cancer.

BALB/c female mice can also be used for footpad or subcutaneous immunization. Each mouse should be injected with murine L cells transfected with human-sema4A (L-sema4A). Mice must be sacrificed several days after injection and popliteal lymph nodes (from footpad immunization) or spleen (from subcut immunization) removed. Cells are then fused with SP2.0 myeloma cells at a ratio of 1 to 1 to generate hybridoma clones using established protocols. Hybridoma clones secreting monoclonal antibody can then be screened for their binding specificity to L-sema4A cells by ELISA assays. Hybridoma supernatants which bind to L-sema4A cells and not L parental cells were further tested for binding on L-sema4A and SUPM2-sema4A cells by flow cytometry analysis.

### Chimeric and Humanized Antibodies

Humanization (also sometimes referred to as “reshaping” or “CDR-grafting) is an established technique for reducing the immunogenicity of monoclonal antibodies from xenogeneic sources (including but not limited to rodents) and for improving their activation of the human immune system. Although the mechanics of producing the engineered monoclonal antibody using the techniques of molecular biology are known, simple grafting of the rodent complementary-determining regions (“CDRs”) into human frameworks does not always reconstitute the binding affinity and specificity of the original monoclonal antibody.

In order to humanize an antibody, the design of the humanized antibody becomes the critical step in reproducing the function of the original molecule. This design includes various choices: the extents of the CDRs, the human frameworks to use and the substitution of residues from the rodent monoclonal antibody into the human framework regions (backmutations). The positions of these backmutations have been identified principally by sequence/structural analysis or by analysis of a homology model of the variable regions' 3D structure.

Recently, phage libraries have been used to vary the amino acids at chosen positions. Similarly, many approaches have been used to choose the most appropriate human frameworks in which to graft the rodent CDRs. Early experiments used a limited subset of well-characterized human monoclonal antibodies (often but not always where the structure was available), irrespective of the sequence identity to the rodent monoclonal antibody (the so-called fixed frameworks approach). Some groups use variable regions with high amino acid sequence identity to the rodent variable regions (homology matching or best-fit); others use consensus or germline sequences while still others select fragments of the framework sequences within each light or heavy chain variable region from several different human monoclonal antibodies. There are also approaches to humanization developed which replace the surface rodent residues with the most common residues found in human monoclonal antibodies (“resurfacing” or “veneering”) and those which use differing definitions of the extents of the CDRs.

### Expression of Humanized Anti-sema4A Antibodies

An antibody, or antibody portion, of the invention can be prepared by recombinant expression of immunoglobulin light and heavy chain genes in a host cell. To express an antibody recombinantly, a host cell is transfected with one or more recombinant expression vectors carrying DNA fragments encoding the immunoglobulin light and heavy chains of the antibody such that the light and heavy chains are expressed in the host cell and, preferably, secreted into the medium in which the host cells are cultured, from which medium the antibodies can be recovered. Standard recombinant DNA methodologies are used to obtain antibody heavy and light chain genes, incorporate these genes into recombinant expression vectors and introduce the vectors into host cells, such as those described in Sambrook, Fritsch and Maniatis (eds), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989) and in U.S. Pat. No. 4,816,397 by Boss et al

Antibodies and antibody fragments and variants can be produced from a variety of animal cells, preferably from mammalian cells, with murine and human cells being particularly preferred. Also, recombinant DNA expression systems could include those that utilize host cells and expression constructs that have been engineered to produce high levels of a particular protein. Such host cells and expression constructs may include Escherichia coli; harboring expression constructs derived from plasmids or viruses (bacteriophage); yeast such as Sacharomyces cerevisieae or Fichia pastoras harboring episomal or chromosomally integrated expression constructs; insect cells and viruses such as Sf9 cells and baculovirus; and mammalian cells harboring episomal or chromosomally integrated (including but not limited to, retroviral) expression constructs (such methods, for example, can be seen from the manuscript Verma et al., J. Immunol. Methods 216:165-181, 1998). Antibodies can also be produced in plants (such methods, for example, can be seen from U.S. Pat. No. 6,046,037; Ma et al., Science 268:716-719, 1995) or by phage display technology (such methods, for example, can be seen from Winter et al., Annu. Rev. Immunol. 12:433-455, 1994).

Human anti-sema4A antibodies that displayed a level of activity and binding specificity/affinity that are desirable can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

In another aspect, the isolated DNA encoding the VH region can be converted to a full-length heavy chain gene by operatively linking the VH-encoding DNA to another DNA molecule encoding heavy chain constant regions (CH1, CH2 and CH3). The sequences of human heavy chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG 1, IgG2, IgG3, IgG4, IgA, IgE, IgM or IgD constant region and any allotypic variant therein as described in Kabat (, Kabat, E. A., et al. (1991) Sequences of Proteins of Immunological Interest, Fifth Edition, U.S. Department of Health and Human Services, NIH Publication No. 91-3242), but most preferably is an IgG 1 or IgG4 constant region. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region.

To create a scFv gene, the VH- and VL-encoding DNA fragments are operatively linked to another fragment encoding a flexible linker, e.g., encoding the amino acid sequence (Gly.sub.4-Ser).sub.3, such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al., Nature (1990) 348:552-554.

Amino acid sequence modification(s) of the antibodies described herein are contemplated. For example, it may be desirable to improve the binding affinity and/or other biological properties of the antibody. Amino acid sequence variants of the antibody are prepared by introducing appropriate nucleotide changes into the antibody nucleic acid, or by peptide synthesis. Such modifications include, for example, deletions from, and/or insertions into and/or substitutions of, residues within the amino acid sequences of the antibody. Any combination of deletion, insertion, and substitution is made to arrive at the final construct, provided that the final construct possesses the desired characteristics. The amino acid alterations may be introduced in the subject antibody amino acid sequence at the time that sequence is made.

A useful method for identification of certain residues or regions of the antibody that are preferred locations for mutagenesis is called “alanine scanning mutagenesis” as described by Cunningham and Wells (1989) Science, 244:1081-1085. Here, a residue or group of target residues are identified (e.g., charged residues such as arg, asp, his, lys, and glu) and replaced by a neutral or negatively charged amino acid (most preferably alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed immunoglobulins are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of the antibody molecule include the fusion to the N- or C-terminus of the antibody to an enzyme (e.g. for ADEPT) or a polypeptide which increases the serum half-life of the antibody. Another type of amino acid variant of the antibody alters the original glycosylation pattern of the antibody. Such altering includes deleting one or more carbohydrate moieties found in the antibody, and/or adding one or more glycosylation sites that are not present in the antibody.

Another type of variant is an amino acid substitution variant. These variants have at least one amino acid residue in the antibody molecule replaced by a different residue. The sites of greatest interest for substitutional mutagenesis include the hypervariable regions, but FR alterations are also contemplated. Conservative substitutions are shown in Table 1 of U.S. Pat. No. 7,812,133, Col. 43, ls. 55 to Col. 44 l. 49, incorporated herein by reference, and under the heading of “preferred substitutions”. If such substitutions result in a change in biological activity, then more substantial changes, denominated “exemplary substitutions” in the Table 1, or as further described below in reference to amino acid classes, may be introduced and the products screened.

Furthermore, substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties: (1) hydrophobic: norleucine, met, ala, val, leu, ile; (2) neutral hydrophilic: Cys, Ser, Thr, Asn, Gln; (3) acidic: asp, glu; (4) basic: his, lys, arg; (5) residues that influence chain orientation: gly, pro; and (6) aromatic: trp, tyr, phe. Non-conversative substitutions will entail exchanging a member of one of these classes for another class.

To express the antibodies, or antibody portions described herein, DNAs encoding partial or full-length light and heavy chains, obtained as described above, are inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present).

The recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

As noted above, in addition to the antibody chain genes, the recombinant expression vectors of the invention carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel; Gene Expression Technology: Methods in Enzymology 185, Academic Press, San Diego, Calif. (1990). It will be appreciated that the design of the expression vector, including the selection of regulatory sequences may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al., U.S. Pat. No. 5,464,758 by Bujard et al. and U.S. Pat. No. 5,654,168 by Bujard et al.

In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr.sup.-host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody. Mammalian host cells for expressing the recombinant antibodies described herein include Chinese Hamster Ovary (CHO cells) (such as dhfr-CHO cells, described in Urlaub and Chasin, (1980) Proc. Natl. Acad. Sci. USA 77:4216-4220, used with a DHFR selectable marker, e.g, as described in R. J. Kaufman and P. A. Sharp (1982) Mol. Biol. 159:601-621), NSO myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or, secretion of the antibody into the culture medium in which the host cells are grown. Antibodies can be recovered from the culture medium using standard protein purification methods.

Host cells can also be used to produce portions of intact antibodies, such as Fab fragments or scFv molecules. It will be understood that variations on the above procedure are within the scope of the present invention. For example, it may be desirable to transfect a host cell with DNA encoding either the light chain or the heavy chain (but not both) of an antibody of this invention. Recombinant DNA technology may also be used to remove some or all of the DNA encoding either or both of the light and heavy chains that is not necessary for binding to sema4A The molecules expressed from such truncated DNA molecules are also encompassed by the antibodies of the invention. In addition, bifunctional antibodies may be produced in which one heavy and one light chain are an antibody of the invention and the other heavy and light chain are specific for an antigen other than sema4A by crosslinking an antibody of the invention to a second antibody by standard chemical crosslinking methods.

Any of the anti-human sema4A antibodies taught herein can be obtained by designing a suitable antigen screening procedure to select for the phage clone of interest followed by construction of a full length anti-human sema4A antibody clone using the Fv sequences from the phage clone of interest and suitable constant region (Fc) sequences described in Kabat et al., Sequences of Proteins of Immunological Interest, Fifth Edition, NIH Publication 91-3242, Bethesda Md. (1991), vols. 1-3.

In this case, the antigen-binding domain of an antibody is formed from two variable (V) regions of about 110 amino acids, one each from the light (VL) and heavy (VH) chains, that both present three hypervariable loops or complementarity-determining regions (CDRs). Variable domains can be displayed functionally on phage, either as single-chain Fv (scFv) fragments, in which VH and VL are covalently linked through a short, flexible peptide, or as Fab fragments, in which they are each fused to a constant domain and interact non-covalently. As used herein, scFv encoding phage clones and Fab encoding phage clones are collectively referred to as “Fv phage clones” or “Fv clones”.

Repertoires of VH and VL genes can be separately cloned by polymerase chain reaction (PCR) and recombined randomly in phage libraries, which can then be searched for antigen-binding clones. Libraries from immunized sources provide high-affinity antibodies to the immunogen without the requirement of constructing hybridomas. Alternatively, the naive repertoire can be cloned to provide a single source of human antibodies to a wide range of non-self and also self antigens without any immunization. Finally, naive libraries can also be made synthetically by cloning the unrearranged V-gene segments from stem cells, and using PCR primers containing random sequence to encode the highly variable CDR3 regions and to accomplish rearrangement in vitro.

Filamentous phage can be used to display antibody fragments by fusion to the minor coat protein pill. The antibody fragments can be displayed as single chain Fv fragments, in which VH and VL domains are connected on the same polypeptide chain by a flexible polypeptide spacer, or as Fab fragments, in which one chain is fused to pIII and the other is secreted into the bacterial host cell periplasm where assembly of a Fab-coat protein structure which becomes displayed on the phage surface by displacing some of the wild type coat proteins.

In general, nucleic acids encoding antibody gene fragments are obtained from immune cells harvested from humans or animals. If a library biased in favor of anti-human sema4A clones is desired, the subject is immunized with sema4A to generate an antibody response, and spleen cells and/or circulating B cells other peripheral blood lymphocytes (PBLs) are recovered for library construction.

A human antibody gene fragment library biased in favor of anti-human sema4A clones is obtained by generating an anti-human sema4A antibody response in transgenic mice carrying a functional human immunoglobulin gene array (and lacking a functional endogenous antibody production system) such that sema4A immunization gives rise to B cells producing human antibodies against sema4A.

Alternatively, the use of spleen cells and/or B cells or other PBLs from an unimmunized donor provides a better representation of the possible antibody repertoire, and also permits the construction of an antibody library using any animal (human or non-human) species in which sema4A is not antigenic.

For libraries incorporating in vitro antibody gene construction, stem cells are harvested from the subject to provide nucleic acids encoding un-rearranged antibody gene segments. The immune cells of interest can be obtained from a variety of animal species, such as human, mouse, rat, lagomorpha, luprine, canine, feline, porcine, bovine, equine, and avian species, etc.

Nucleic acid encoding antibody variable gene segments (including VH and VL segments) are recovered from the cells of interest and amplified. In the case of rearranged VH and VL gene libraries, the desired DNA can be obtained by isolating genomic DNA or mRNA from lymphocytes followed by polymerase chain reaction (PCR) with primers matching the 5′ and 3′ ends of rearranged VH and VL genes, thereby making diverse V gene repertoires for expression.

Repertoires of antibody fragments can be constructed by combining VH and VL gene repertoires together in several ways. Each repertoire can be created in different vectors, and the vectors recombined in vitro, or in vivo by combinatorial infection, e.g., the loxP system. The in vivo recombination approach exploits the two-chain nature of Fab fragments to overcome the limit on library size imposed by E. coli transformation efficiency. Naive VH and VL repertoires are cloned separately, one into a phagemid and the other into a phage vector. The two libraries are then combined by phage infection of phagemid-containing bacteria so that each cell contains a different combination and the library size is limited only by the number of cells present (about 10¹² clones). Both vectors contain in vivo recombination signals so that the VH and VL genes are recombined onto a single replicon and are co-packaged into phage virions. These huge libraries provide large numbers of diverse antibodies of good affinity (K_{d}⁻¹ of about 10⁻⁸ M).

Alternatively, the repertoires may be cloned sequentially into the same vector, or assembled together by PCR and then cloned. PCR assembly can also be used to join VH and VL DNAs with DNA encoding a flexible peptide spacer to form single chain Fv (scFv) repertoires. In yet another technique, “in cell PCR assembly” is used to combine VH and VL genes within lymphocytes by PCR and then clone repertoires of linked genes.

The antibodies produced by naive libraries (either natural or synthetic) can be of moderate affinity (K_{d}⁻¹ of about 10⁶ to 10⁷ M¹), but affinity maturation can also be mimicked in vitro by constructing and reselecting from secondary libraries.

Additionally, affinity maturation can be performed by randomly mutating one or more CDRs, e.g. using PCR with primers carrying random sequence spanning the CDR of interest, in selected individual Fv clones and screening for higher affinity clones. WO 9607754 (published 14 Mar. 1996) described a method for inducing mutagenesis in a complementarity determining region of an immunoglobulin light chain to create a library of light chain genes. Another effective approach is to recombine the VH or VL domains selected by phage display with repertoires of naturally occurring V domain variants obtained from unimmunized donors and screen for higher affinity in several rounds of chain reshuffling. This technique allows the production of antibodies and antibody fragments with affinities in the 10⁻⁹ M range.

### Making an Anti-Human Sema4A Monoclonal Antibody and Subsequent Antibody Production of the Same

The anti-human sema4A monoclonal antibodies of the invention can be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. Preferred myeloma cells are those that fuse efficiently, support stable high-level production of antibody by the selected antibody-producing cells, and are sensitive to a medium.

Culture medium in which hybridoma cells are growing is assayed for production of monoclonal antibodies directed against human sema4A. The binding specificity of monoclonal antibodies can be produced by hybridoma cells can be determined by immunoprecipitation or by an in vitro binding assay, such as radioimmunoassay (RIA) or enzyme-linked immunoadsorbent assay (ELISA). The binding affinity of the monoclonal antibody can, for example, be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods. Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Specific monoclonal antibodies that bind to human sema4A are provided in Tables I through IV immediately below.

TABLE I
Monoclonal Antibodies Against Human Sema4A

| Clone | IG | FACS | | Histology |
|---|---|---|---|---|
| Number | isotype | staing | blocking | staining |
| 126-25B-1 | IgG1 | yes | yes | NO |
| 126-28-1 | IgG2a | yes | yes | yes |
| 126-31B-1 | IgG2a | yes | NO | NO |
| 126-63-1 | IgG1 | yes | yes | NO |
| 126-65B-1 | IgG2b + IgG1 | yes | yes | NO |
| 161-15B | IgG1 | yes | yes | NO |
| 161-18B | IgG1 | yes | yes | yes |
| 161-33 | IgG2B | yes | NO | NO |
| 161-35 | IgG2A | yes | yes | NO |
| 161-44 | IgG2B | yes | yes | yes |
| 161-49 | IgG2B | yes | yes | NO |
| 161-51 | IgG1 | yes | yes | NO |
| 161-66 | IgG2A | yes | yes | NO |
| 161-70 | IgG2A | yes | yes | yes |
| 161-90 | IgG2A | yes | yes | yes |
| 161-96B | IgG2B | yes | yes | NO |
| 161-110B | IgG2A | yes | yes | NO |
| 161-117 | IgG2A | yes | yes | NO |
| 161-118A | IgG2A | yes | NO | NO |

TABLE II
Monoclonal Antibody Clones that Work
on FACS Staining and Blocking

| Clone | | FACS | |
|---|---|---|---|
| Number | IG isotype | staining | Blocking |
| 126-25B-1 | IgG1 | yes | yes |
| 126-28-1 | IgG2a | yes | yes |
| 126-63-1 | IgG1 | yes | yes |
| 126-65B-1 | IgG2b + IgG1 | yes | yes |
| 161-15B | IgG1 | yes | yes |
| 161-18B | IgG1 | yes | yes |
| 161-35 | IgG2A | yes | yes |
| 161-44 | IgG2B | yes | yes |
| 161-49 | IgG2B | yes | yes |
| 161-51 | IgG1 | yes | yes |
| 161-66 | IgG2A | yes | yes |
| 161-70 | IgG2A | yes | yes |
| 161-90 | IgG2A | yes | yes |
| 161-96B | IgG2B | yes | yes |
| 161-110B | IgG2A | yes | yes |
| 161-117 | IgG2A | yes | yes |

TABLE III
Clones work on FACS staining, blocking and histology staining

| Clone | IG | FACS | | Histology |
|---|---|---|---|---|
| Number | Isotype | staining | Blocking | staining |
| 126-28-1 | IgG2a | yes | yes | yes |
| 161-18B | IgG1 | yes | yes | yes |
| 161-44 | IgG2B | yes | yes | yes |
| 161-70 | IgG2A | yes | yes | yes |
| 161-90 | IgG2A | yes | yes | yes |

TABLE IV
Clones that work on FACS staining only

| Clone | IG | FACS | | Histology |
|---|---|---|---|---|
| Number | isotype | staining | Blocking | Staining |
| 126-31B-1 | IgG2a | yes | NO | NO |
| 161-33 | IgG2B | yes | NO | NO |
| 161-118A | IgG2A | yes | NO | NO |

### Cloning and Sequencing of the Variable Region Genes of Mouse 161-51-1, 161-70-1 and 161-15B-1 Monoclonal Antibodies

Mouse 161-51-1, 161-70-1 and 161-15B1 hybridoma cells were grown in RPMI-1640 medium containing 10% fetal bovine serum (HyClone, Logan, Utah) and 1 mM sodium pyruvate at 37° C. in a 7.5% CO₂ incubator. Total RNA was extracted from approximately 8×10⁶ hybridoma cells using TRIzol reagent (Invitrogen, Carlsbad, Calif.) according to the supplier's protocol. Oligo dT-primed cDNA was synthesized using the SMARTer RACE cDNA Amplification Kit (Clontech, Mountain View, Calif.) following the supplier's protocol. The variable region cDNAs for 161-51-1, 161-70-1 and 161-15B-1 heavy and light chains were amplified by polymerase chain reaction (PCR) with Phusion DNA polymerase (New England Biolabs, Beverly, Mass.) using 3′ primers that anneal respectively to the mouse gamma and kappa chain constant regions, and a 5′-RACE primer (Universal Primer A Mix or Nested Universal Primer A) provided in the SMARTer RACE cDNA Amplification Kit. For PCR amplification of heavy chain variable region (VH) of 161-51-1 and 161-15B-1, the 3′ primer binding to the mouse gamma-1 gene has the sequence 5′-GCCAGTGGATAGACAGATGG-3′ (MCG1). For PCR amplification of 161-70-1 VH, the 3′ primer binding to the mouse gamm-2a gene has the sequence 5′-GCCAGTGGATAGACCGATGG-3′ (MCG2A). For PCR amplification of light chain variable region (VL) for all three antibodies, the 3′ primer has the sequence 5′-GATGGATACAGTTGGTGCAGC-3′ (MCK). The amplified VH and VL cDNAs were cloned into the pCR-Blunt II-TOPO vector (Invitrogen) for sequence determination. DNA sequencing of the variable regions was carried out at Tocore (Menlo Park, Calif.). Several heavy and light chain clones were sequenced for each antibody, and unique sequences homologous to typical mouse heavy and light chain variable regions were identified.

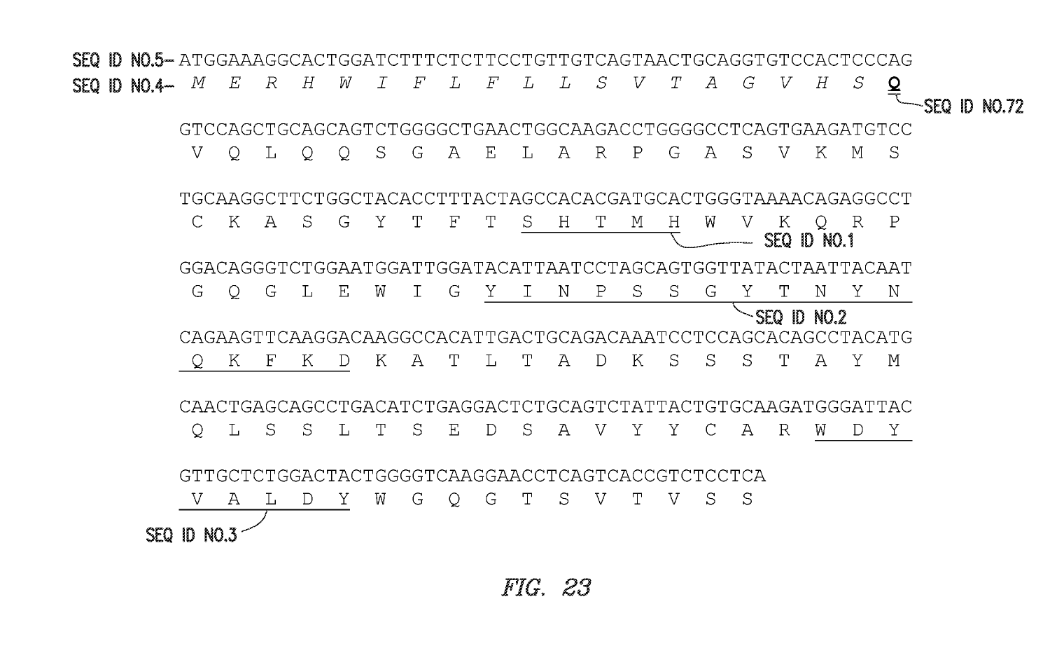

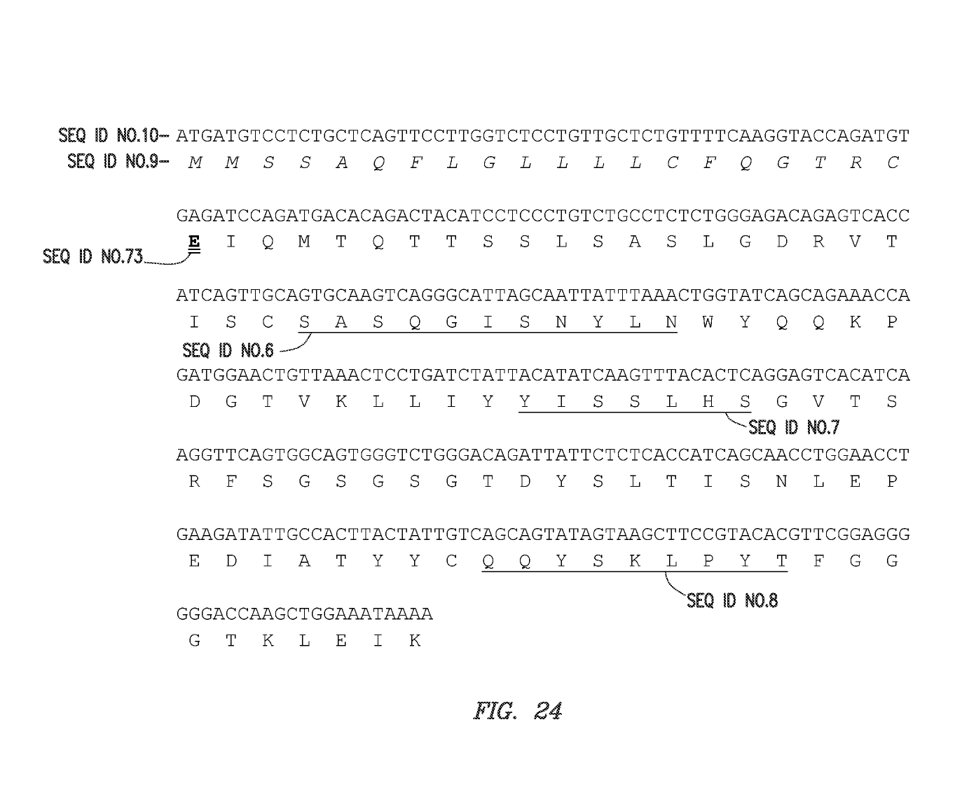

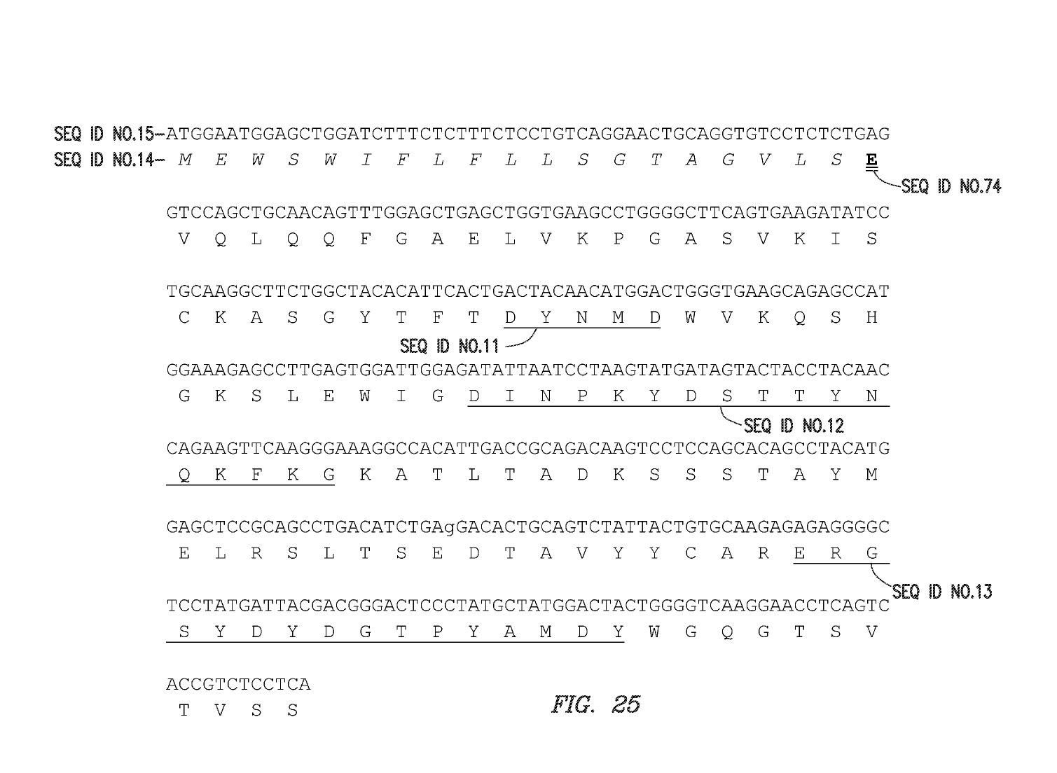

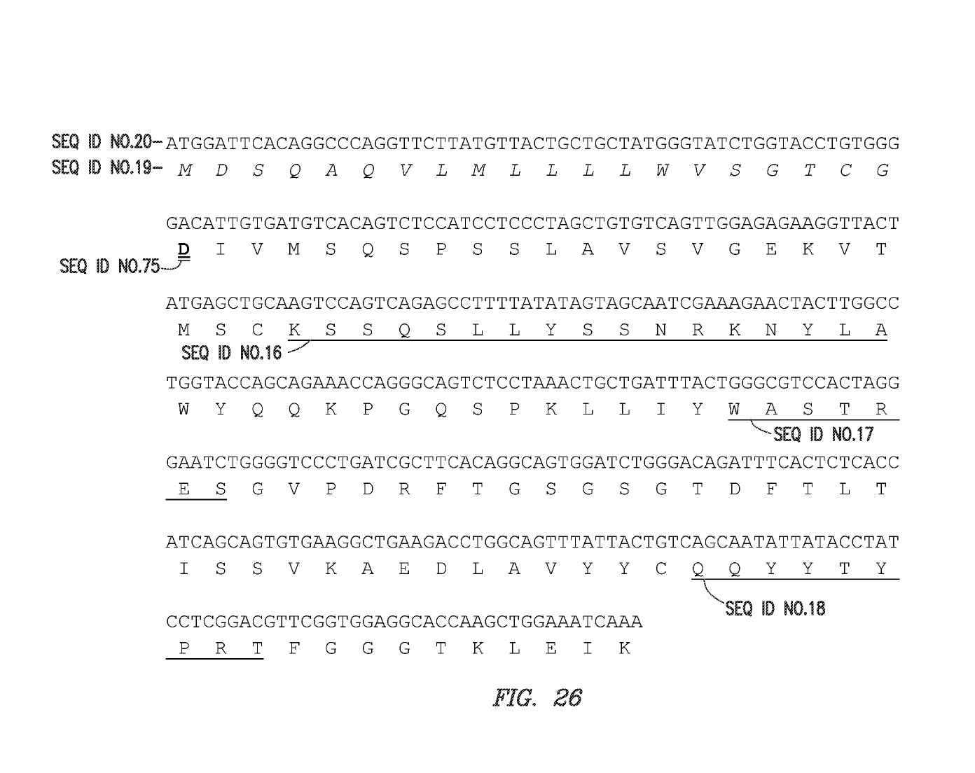

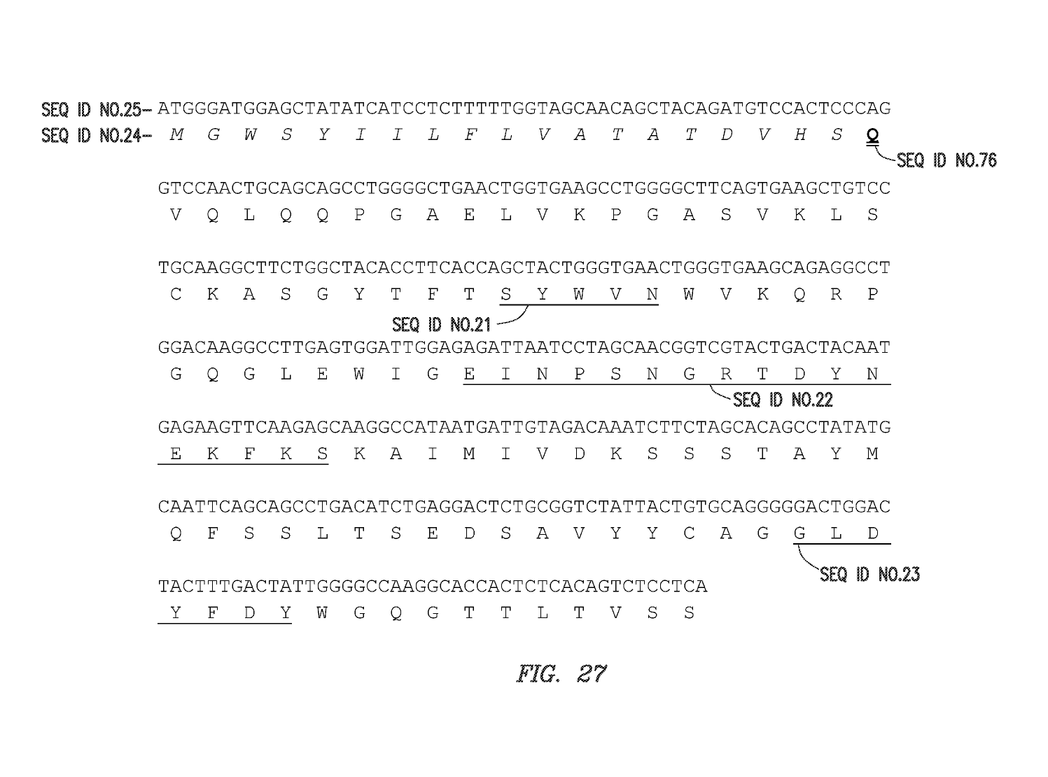

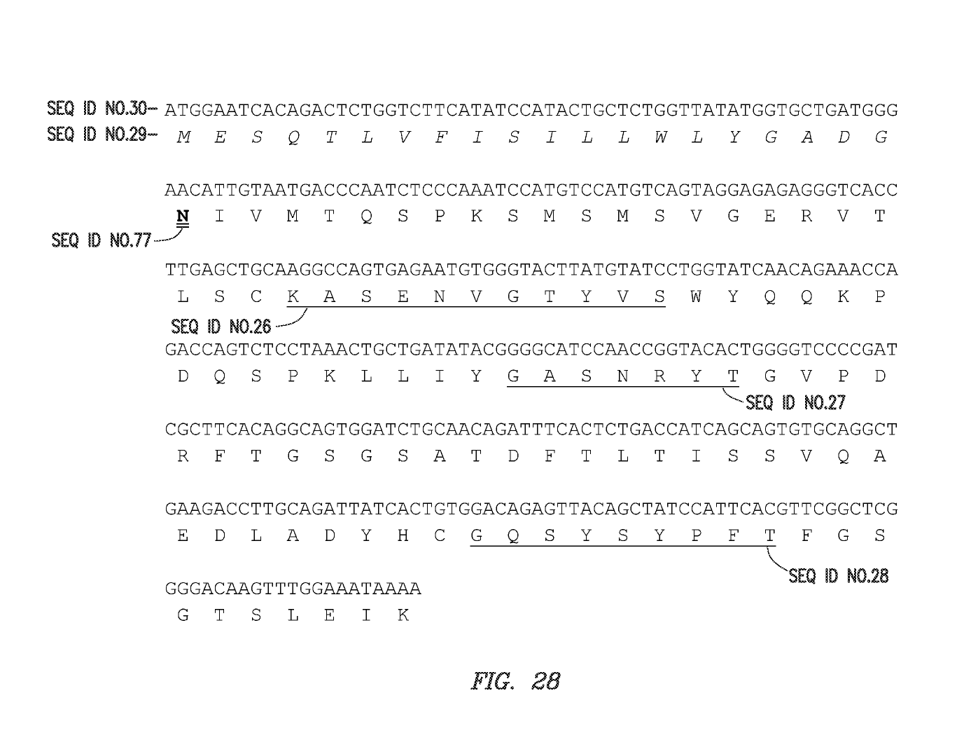

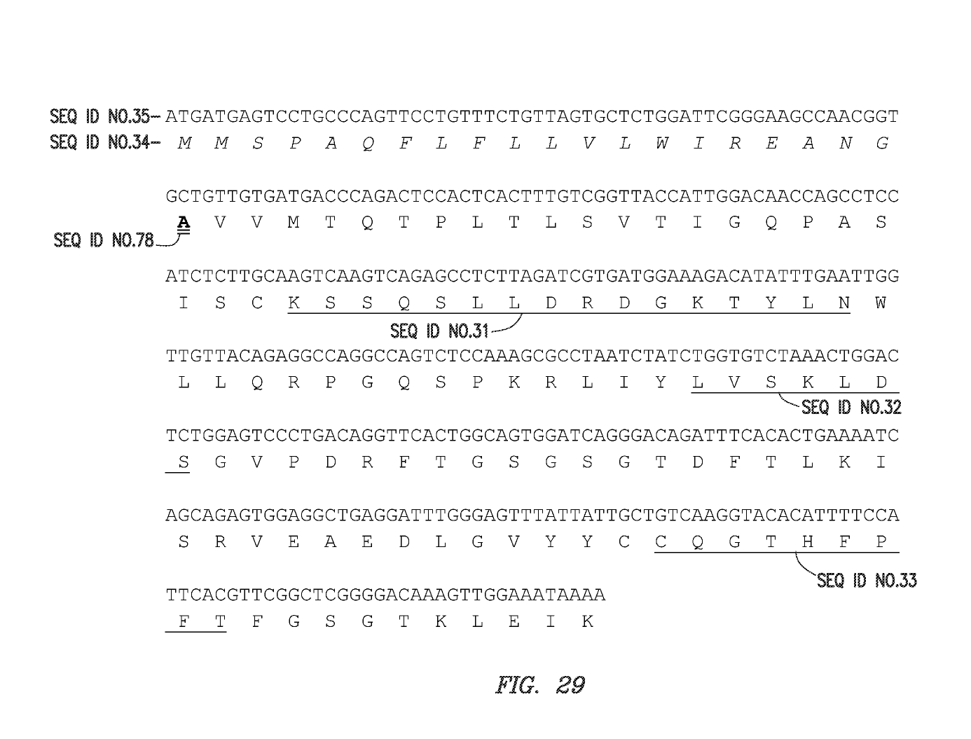

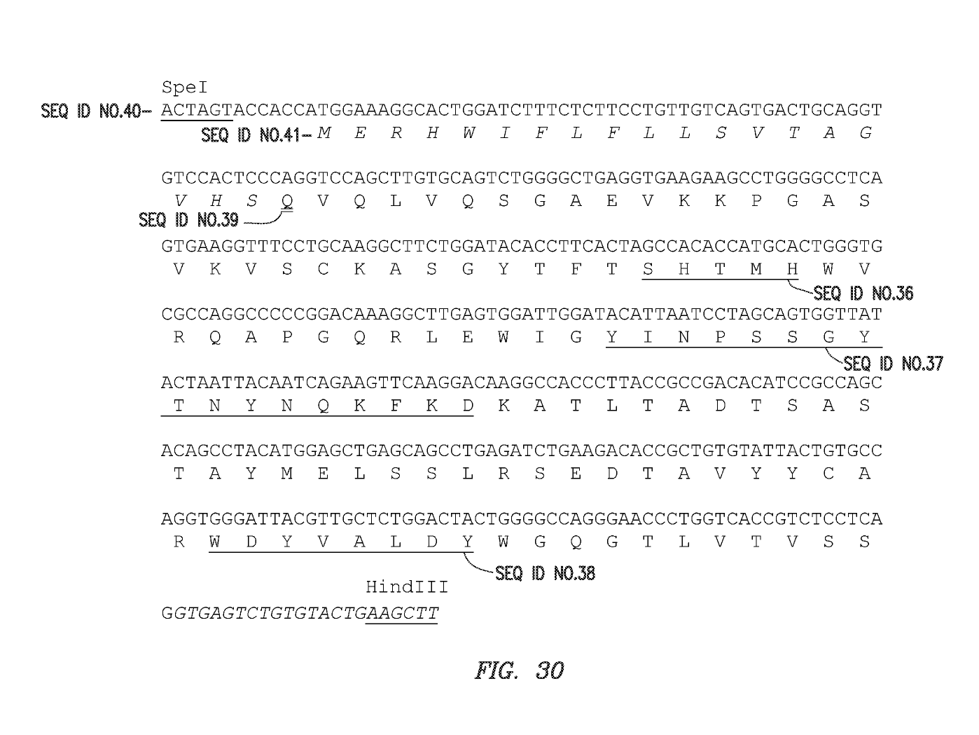

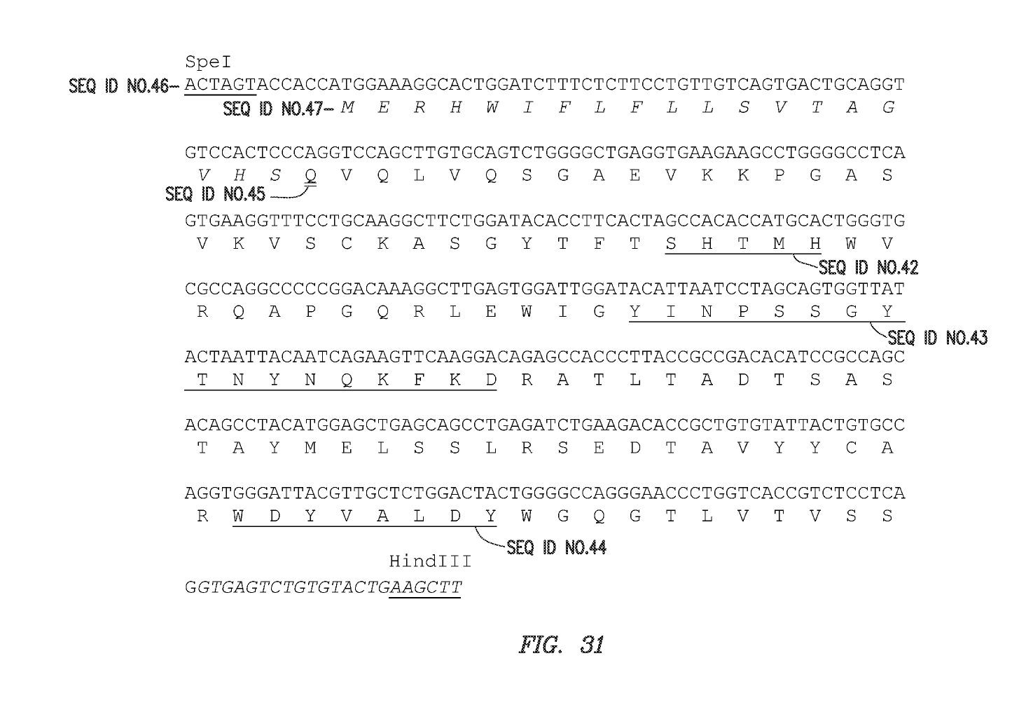

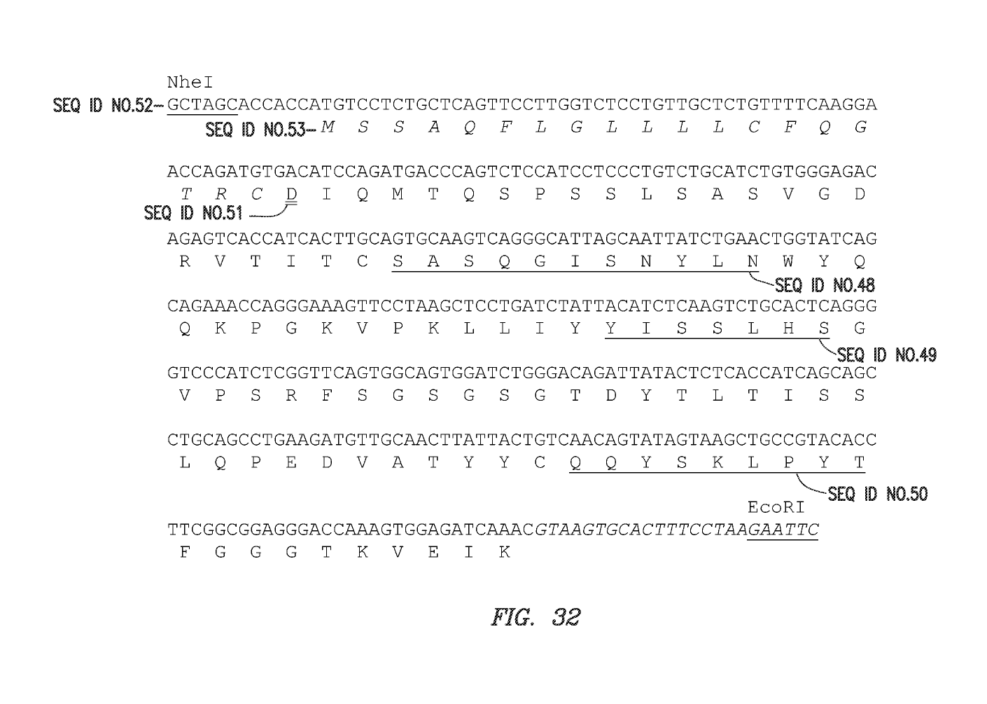

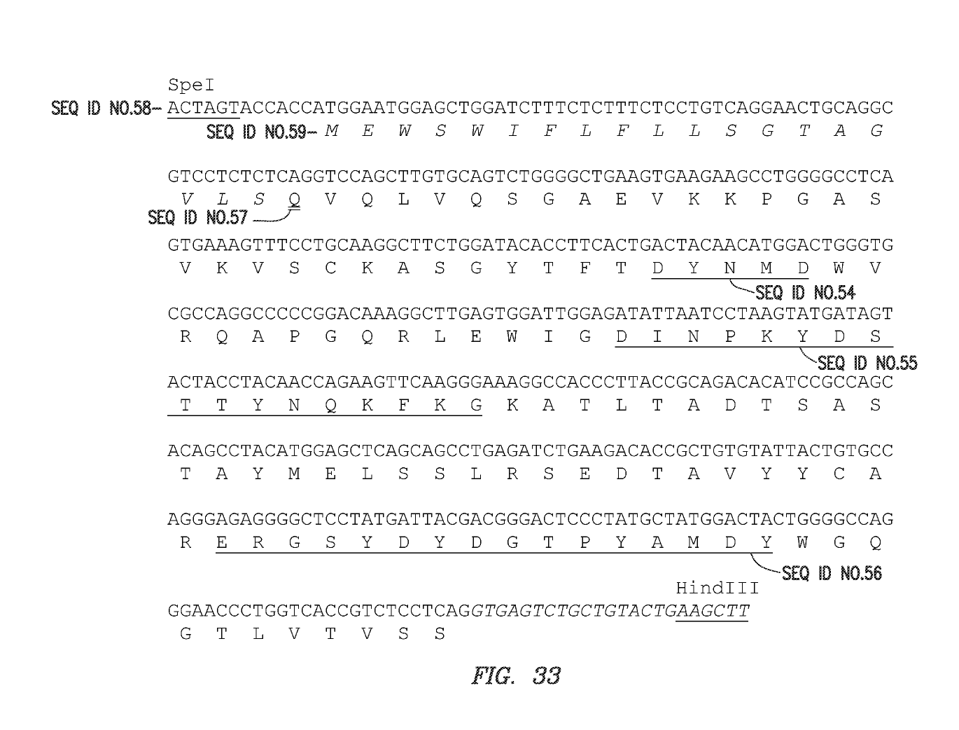

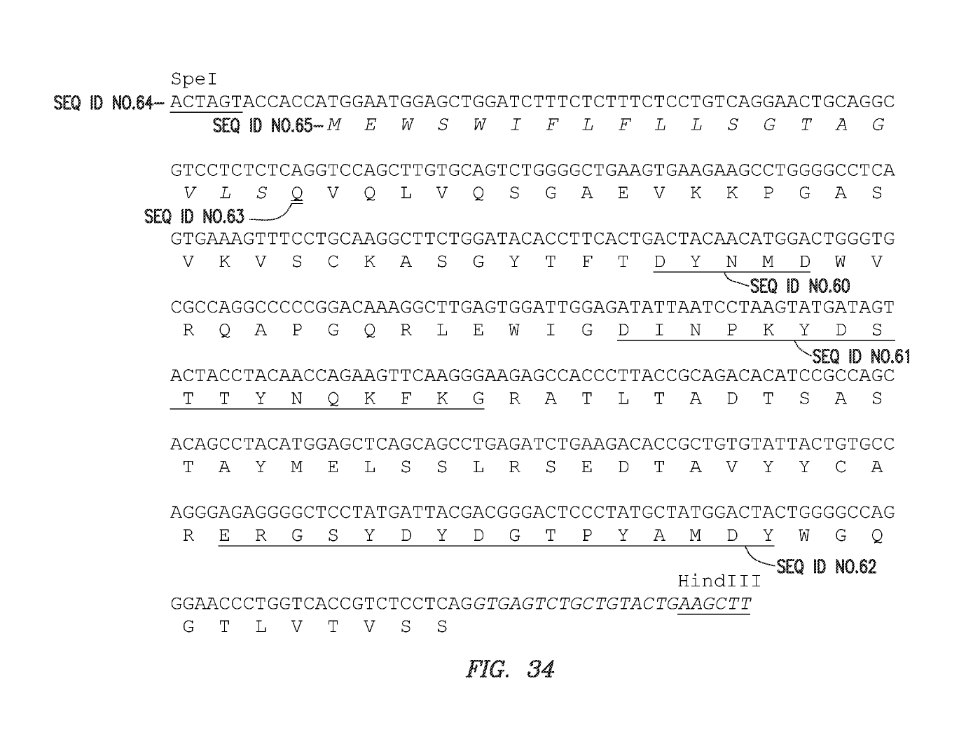

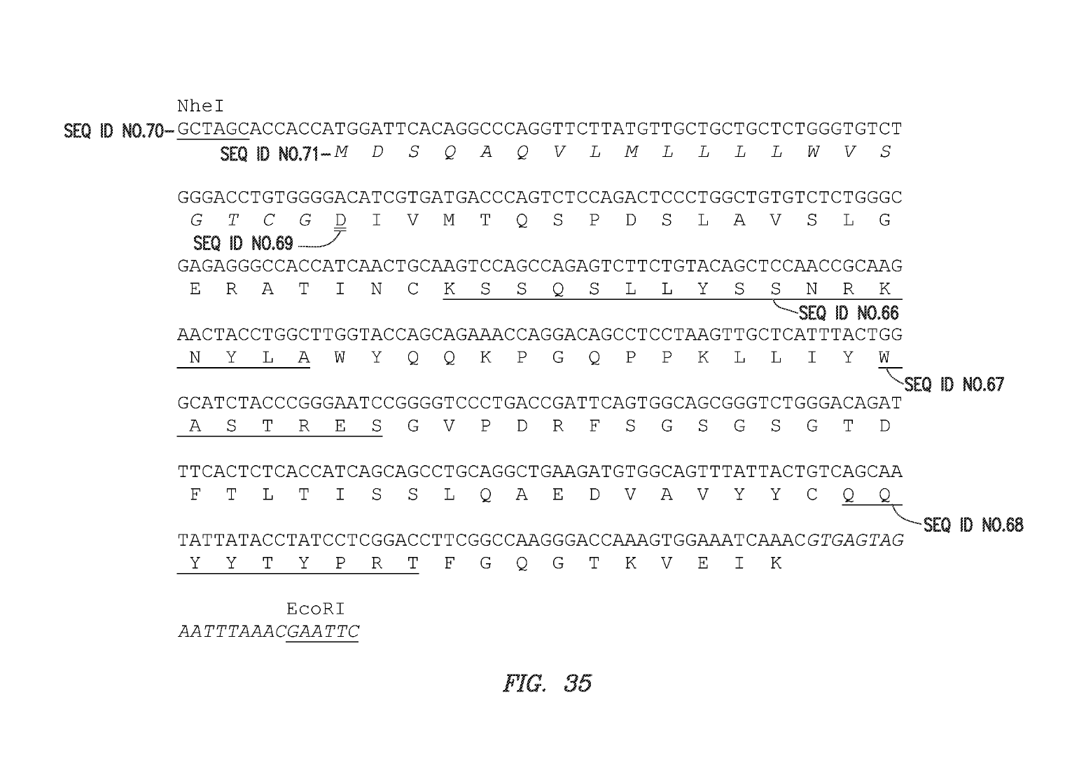

The consensus cDNA sequences of 161-51-1 VH and VL along with deduced amino acid sequences are shown in FIGS. 23 and 24, respectively. In each figure, the signal peptide sequence is in italic and CDR sequences according to the definition of Kabat et al. (Sequences of Proteins of Immunological Interests, Fifth edition, NIH Publication No. 91-3242, U.S. Department of Health and Human Services, 1991) are underlined. Likewise, the consensus cDNA sequences of 161-70-1 VH and VL along with deduced amino acid sequences are shown in FIGS. 25 and 26, respectively. The consensus cDNA sequence of 161-15B-1 VH along with the deduced amino acid sequence is shown in FIG. 27. As for 161-15B-1 VL, two productive VL sequences were obtained. Seven out of nine productive VL sequences derived from 161-15B-1 hybridoma cells had the sequence shown as 161-15B-1 VL1 in FIG. 28. Two out of the nine productive VL sequences had the sequence shown as 161-15B-1 VL2 in FIG. 29. Only one kind of productive VH sequence shown in FIG. 27 was obtained from 161-15B-1 hybridoma cells. Table V provided immediately below provides information related to humanized antibodies described herein.

TABLE V
Humanized mAbs
anti-Sema4A mAb humanization (acid elution)

| Clone name | Lot number |
|---|---|
| ChLB51 | Lot 12/6/11 |
| HuLB51 #1 | Lot 1/12/12, |
| HuLB51 #2 | Lot 1/5/12, |
| ChLB70, | Lot 12/30/11, |
| HuLB70 #1 | Lot 1/13/12, |
| HuLB70 #2 | Lot 1/12/12, |

ChLB51: chimeric 161-51-1;
HuLB51-1 and HuLB51-2: two forms of humanized 161-51-1;
ChLB70: chimeric 161-70-1;
HuLB70-1 and HuLB70-2: two forms of humanized 161-70-1.
*HuLB51-1 and HuLB51-2 are different from each other by one aa in the VH region
*HuLB70-1 and HuLB70-2 are different from each other by one aa in the VH region

Antibody Production Based on Isolated Polynucleotide Sequences from Antibody Producing Cells

Polynucleotide sequences encoding polypeptide components of the antibody can be obtained using standard recombinant techniques. As noted above, desired polynucleotide sequences may be isolated and sequenced from antibody producing cells such as hybridoma cells. Alternatively, polynucleotides can be synthesized using nucleotide synthesizer or PCR techniques.

Once obtained, sequences encoding the polypeptides are inserted into a recombinant vector capable of replicating and expressing heterologous polynucleotides in prokaryotic or eukaryotic hosts. Many vectors that are available and known in the art can be used for this purpose. Selection of an appropriate vector will depend mainly on the size of the nucleic acids to be inserted into the vector and the particular host cell to be transformed with the vector.

Each vector contains various components, depending on its function (amplification or expression of heterologous polynucleotide, or both) and its compatibility with the particular host cell in which it resides. The vector components generally include, but are not limited to: an origin of replication, a selection marker gene, a promoter, a ribosome binding site (RBS), a signal sequence, the heterologous nucleic acid insert and a transcription termination sequence.

Host cells can be transformed, transduced or transfected with expression vectors and/or nucleic acids and cultured in media modified as appropriate for inducing promoters, selecting transformants, or amplifying the genes encoding the desired sequences. For prokaryotic cells, standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75.

When generating antibodies using eukaryotic host cells, the vector components generally include, but are not limited to, one or more of the following: a signal sequence, an origin of replication, one or more marker genes, an enhancer element, a promoter, and a transcription termination sequence. A vector for use in a eukaryotic host cell may also contain a signal sequence or other polypeptide having a specific cleavage site at the N-terminus of the mature protein or polypeptide of interest. The heterologous signal sequence selected preferably is one that is recognized and processed (i.e., cleaved by a signal peptidase) by the host cell. In mammalian cell expression, mammalian signal sequences as well as viral secretory leaders, for example, the herpes simplex gD signal, are available. The DNA for such precursor region is ligated in reading frame to DNA encoding the antibody. Generally, an origin of replication component is not needed for mammalian expression vectors. For example, the SV40 origin may typically be used only because it contains the early promoter.

Expression and cloning vectors used in eukaryotic host cells may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, where relevant, or (c) supply critical nutrients not available from complex media. One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin. Another example of suitable selectable markers for mammalian cells are those that enable the identification of cells competent to take up the antibody nucleic acid, such as DHFR, thymidine kinase, metallothionein-I and -II, preferably primate metallothionein genes, adenosine deaminase, ornithine decarboxylase, etc.

The antibodies and methods that are described herein can be used to prevent or treat inflammatory diseases and conditions, such as osteoarthritis, Rheumatoid arthritis, Crohn's disease, ulcerative colitis, and auto-immune diseases such as lupus and mixed auto-immune disease. For example, the antibodies described herein may be useful in treating a variety of autoimmune and inflammatory disease comprising the step of administering a therapeutically effective amount of the antibody to a subject in need thereof, wherein the autoimmune disease or inflammatory disease is any one or more of the following diseases: insulin-dependent diabetes mellitus (IDDM), diabetes mellitus, multiple sclerosis, experimental autoimmune encephalomyelitis, acute disseminated encephalomyelitis, arthritis, rheumatoid arthritis, experimental autoimmune arthritis, myasthenia gravis, thyroiditis, Hashimoto's disease, primary myxedema, thyrotoxicosis, pernicious anemia, autoimmune atrophic gastritis, Addison's disease, premature menopause, male infertility, juvenile diabetes, Goodpasture's syndrome, pemphigus vulgaris, pemphigoid, sympathetic ophthalmia, phacogenic uveitis, autoimmune haemolyticanaemia, idiopathic leucophenia, primary biliary cirrhosis, active chronic hepatitis Hbₛ₋ᵥₑ, cryptogenic cirrhosis, ulcerative colitis, Sjogren's syndrome, scleroderma, Wegener's granulomatosis, Poly/Dermatomyositis, discoid LE, systemic Lupus erythematosus, Chron's disease, psoriasis, Ankylosingspondylitisis, Antiphospholipid antibody syndrome, Aplastic anemia, Autoimmune hepatitis, Coeliac disease, Graves' disease, Guillain-Barre syndrome (GBS), Idiopathic thrombocytopenic purpura, Opsoclonus myoclonus syndrome (OMS). Optic neuritis, ORd's thyroiditis, Pemphigus, Polyarthritis, Primary biliary cirrhosis, Reiter's syndrome, Takayasu's, Temporal arteritis, Warm autoimmune hemolytic anemia, Wegener's granulomatosis, Alopecia universalis, Behcet's disease, Chagas' disease, Chronic fatigue syndrome, Dysautonomia, Endometriosis, Hidradenitis suppurativa, Interstitial cystitis, Neuromyotonia, Sarcoidosis, Scleroderma, Ulcerative colitis, Vitiligo, Vulvodynia, inflammatory skin diseases, allergic contact dermatitis, H. pylory gastritis, chronic nasal inflammatory disease, arteriosclerosis and graft versus host disease.

More specifically, an “autoimmune disease” as referred herein is a disease or disorder arising from and directed against an individual's own tissues or organs or a co-segregate or manifestation thereof or resulting condition there from. Autoimmune disease may refer to a condition that results from, or is aggravated by, the production by B cells of antibodies that are reactive with normal body tissues and antigens. Also, an autoimmune disease is one that may involve the secretion of an autoantibody that is specific for an epitope from a self antigen (e.g. a nuclear antigen).

Autoimmune diseases or disorders that are treatable and/or preventable by any one or more of the antibodies described herein include, but are not limited to, arthritis (rheumatoid arthritis such as acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis, x-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica (NMO), inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), bowel inflammation, pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, proliferative nephritis, autoimmune polyglandular endocrine failure, balanitis including balanitis circumscripta plasmacellularis, balanoposthitis, erythema annulare centrifugum, erythema dyschromicum perstans, eythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema including allergic or atopic eczema, asteatotic eczema, dyshidrotic eczema, and vesicular palmoplantar eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, systemic lupus erythematosus (SLE) such as cutaneous SLE or subacute cutaneous SLE, neonatal lupus syndrome (NLE), and lupus erythematosus disseminatus, juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, diabetic retinopathy, diabetic nephropathy, diabetic large-artery disorder, immune responses associated with acute and delayed hypersensitivity mediated by cytokines and T-lymphocytes, tuberculosis, sarcoidosis, granulomatosis including lymphomatoid granulomatosis, Wegener's granulomatosis, agranulocytosis, vasculitides, including vasculitis, large-vessel vasculitis (including polymyalgia rheumatica and giant-cell (Takayasu's) arteritis), medium-vessel vasculitis (including Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis, such as Churg-Strauss vasculitis or syndrome (CSS) and ANCA-associated small-vessel vasculitis, temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, Alzheimer's disease, Parkinson's disease, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Behcet's disease/syndrome, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, thermal injury, preeclampsia, an immune complex disorder such as immune complex nephritis, antibody-mediated nephritis, polyneuropathies, chronic neuropathy such as IgM polyneuropathies or IgM-mediated neuropathy, thrombocytopenia (as developed by myocardial infarction patients, for example), including thrombotic thrombocytopenic purpura (TTP), post-transfusion purpura (PTP), heparin-induced thrombocytopenia, and autoimmune or immune-mediated thrombocytopenia such as idiopathic thrombocytopenic purpura (ITP) including chronic or acute ITP, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant-cell hepatitis, chronic active hepatitis or autoimmune chronic active hepatitis, lymphoid interstitial pneumonitis (LIP), bronchiolitis obliterans (non-transplant) vs NSIP, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal or segmental or focal segmental glomerulosclerosis (FSGS), endocrine ophthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia greata, alopecia totalis, CREST syndrome (calcinosis, Raynaud's phenomenon, esophageal dysmotility, sclerodactyl), and telangiectasia), male and female autoimmune infertility, e.g., due to anti-spermatozoan antibodies, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, parasitic diseases such as leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, SCID, acquired immune deficiency syndrome (AIDS), echovirus infection, sepsis, endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant-cell polymyalgia, chronic hypersensitivity pneumonitis, keratoconjunctivitis sicca, epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders, asperniogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, non-malignant thymoma, vitiligo, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, autoimmune polyglandular syndrome type I, adult-onset idiopathic hypoparathyroidism (AOIH), cardiomyopathy such as dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as cosinophilia, pulmonary infiltration eosinophilia, cosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, and disease accompanying vascularization, allergic hypersensitivity disorders, glomerulonephritides, reperfusion injury, ischemic re-perfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, and endometriosis.

The antibodies described herein may have a variety of academic, medical and commercial uses. The antibodies may be used in different types of diagnostic tests, for example, to detect a wide variety of diseases or the presence of drugs (pharmaceuticals), toxins or other proteins including hormones, either in vitro or in vivo. The antibodies described herein may be useful in testing for disease, for example, in serum or blood of patients. The disease may including Human Sema4A related diseases or disease or indications not related to Human Sema4A including various cancers, inflammatory or autoimmune disease. Antibodies may also be used in the radioimmuno-detection and radioimmuno-therapy of cancer, and some new testing methods can utilize these described antibodies to target only the cell membranes of specific cell types, i.e., cancer.

The antibodies described herein could be made part of a kit or other diagnostic package. As such, provided herein is a diagnostic kit, or article of manufacture for use with the pretreatment method herein. The diagnostic kit may comprise any one or more of the following: antagonist/antibody/drug reference material; positive control neutralizing antibody (preferably goat of cyno monkey); Protein A+G column (e.g. Protein A/G column); delipidation reagent; immunoglobulin affinity purification buffer(s) (for example binding, elution and neutralization buffers); complement serum; assay diluent for cells; instruction manual or literature; vial of frozen cells (for example, WIL2 cells); cell labeling reagent (such as CELL TITER GLO®), etc. By way of example, the diagnostic kit may include but is not limited to: (a) delipidation reagent; (b) buffers (e.g. binding and elution buffers) for affinity purification of immunoglobulins; and (c) instruction manual instructing the user of the diagnostic kit to use the kit to pre-treat a biological sample from an autoimmune disease or cancer subject prior to conducting a cell based bioassay (such as a neutralizing antibody assay) on the sample (e.g. to avoid the problem of serum interference). The diagnostic kit optionally further comprises any one or more of: drug reference material, positive control neutralizing antibody, complement serum, assay diluent for cells, and cell labeling reagent, etc.

The antibodies and other discoveries described herein also provide for high throughput screening methods.

### Example I

### CD4+CD11c+ mDC Express Higher Sema4A than Other Subsets in Human PBMC

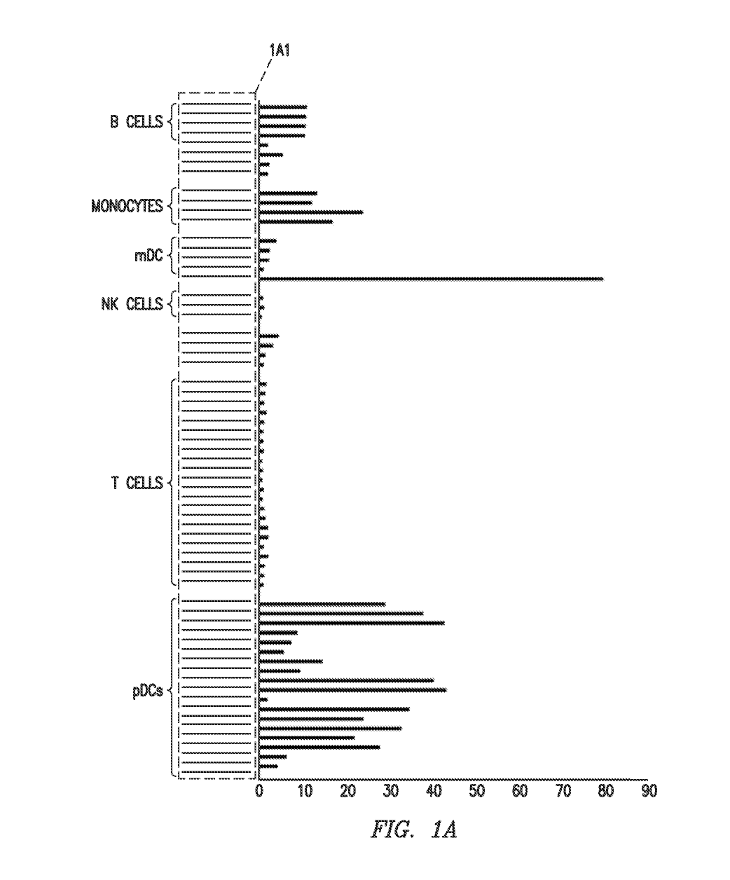

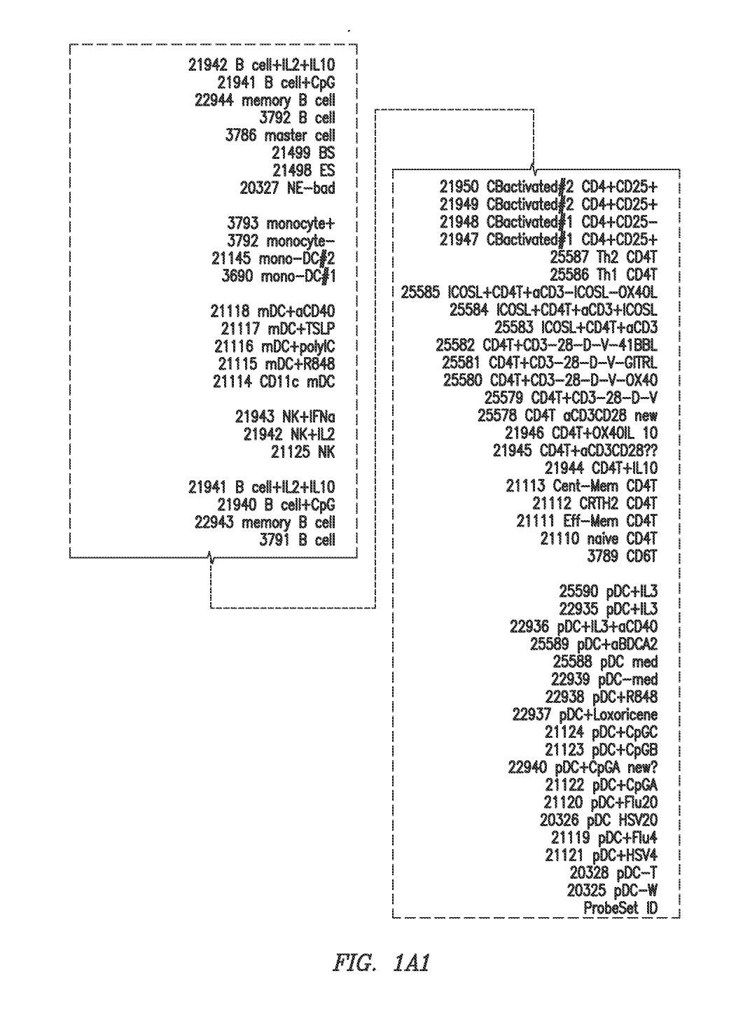

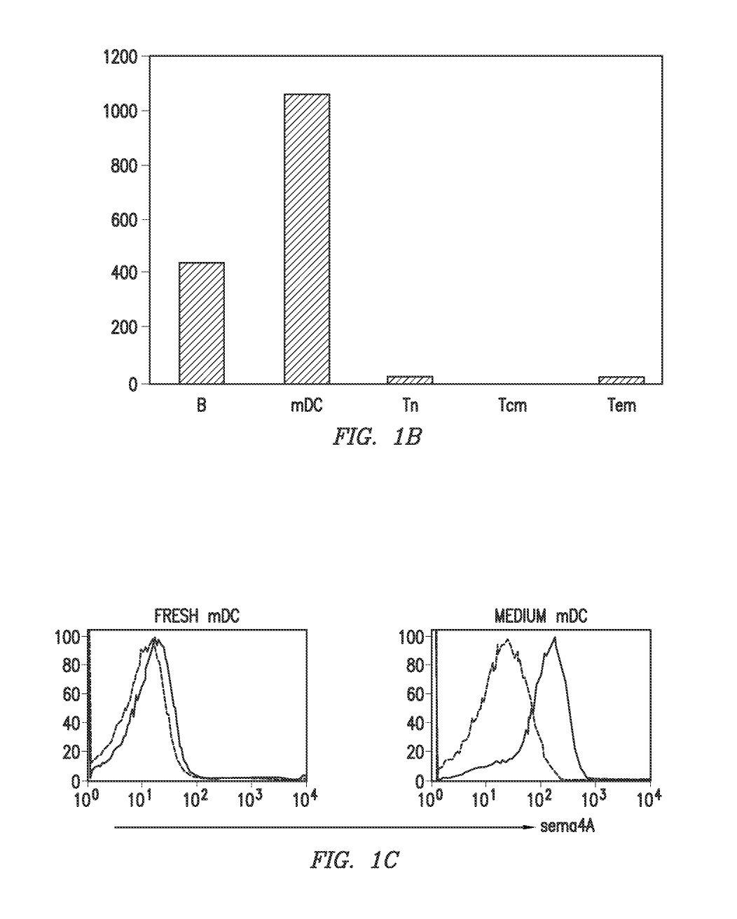

Microarray gene expression analysis shows that among human PBMC, comparing with other subsets including B cell, monocytes, NK cells, T cells and pDCs, CD4+CD11c+ mDC express high level of Sema4A (FIG. 1A). As shown in FIG. 1B, this was confirmed by Q-PCR analyses. Using a mouse anti-human Sema4A mAb generated in our lab and flowcytometry, we found that resting CD4+CD11c+ mDC express surface Sema4A, and the medium cultured mDC express high level surface Sema4A (FIG. 1C).

### Example II

### Human Myeloid DC and Germinal Center B Cells in Human Tonsil Express High sema4A

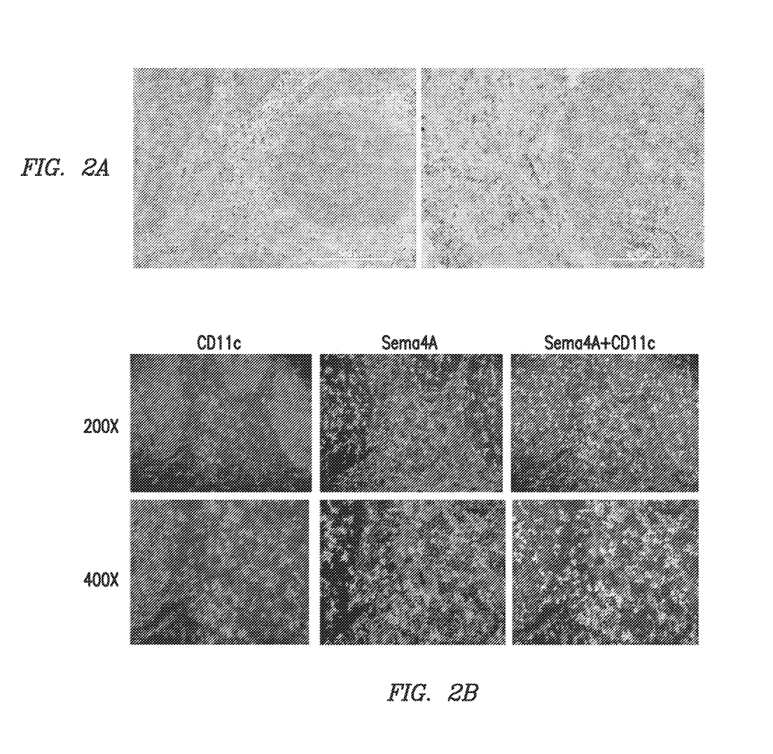

As shown in FIG. 2A, using mouse monoclonal antibodies against human Sema4A, we performed Immunohistology staining on frozen section of human tonsils shows that Sema4A is expressed by germinal center B cells and DCs in the interfollicular areas. As shown in FIG. 2B, double immunofluorescence staining with anti-CD11c (red) and Sema4A (green) further shows that germinal center B cells and a subset of CD11c+ DCs in the interfollicular area express Sema4A.

### Example III

### Sema4A Involves in mDC and T Cell Interaction

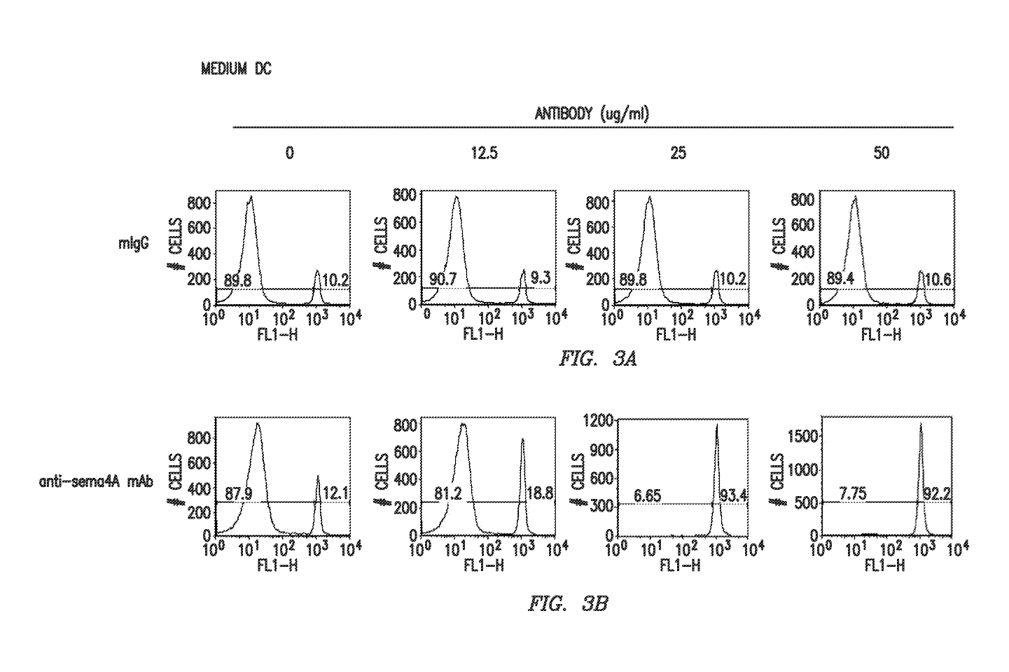

CD4+CD11c+ mDCs are isolated from human PBMC by cell sorting, cultured with medium for 19 hrs, then co-cultured with CFSE-labeled allogeneic naïve CD4+T cells at ratio of T:mDC is 5:1 for 7 days, in the presence of anti-Sema4A blocking mAb or mIgG. In FIG. 3B, according to CFSE dilution, T cell proliferation induced by allogenic mDc which has been pre-treated with medium can be suppressed by anti-sema4A neutralizing mAb synergistically. This phenomenon cannot be observed in mIgG control group, as shown in FIG. 3A.

### Example IV

### Sema4A Co-Stimulates the Proliferation of Human CD4+ Naïve T Cells

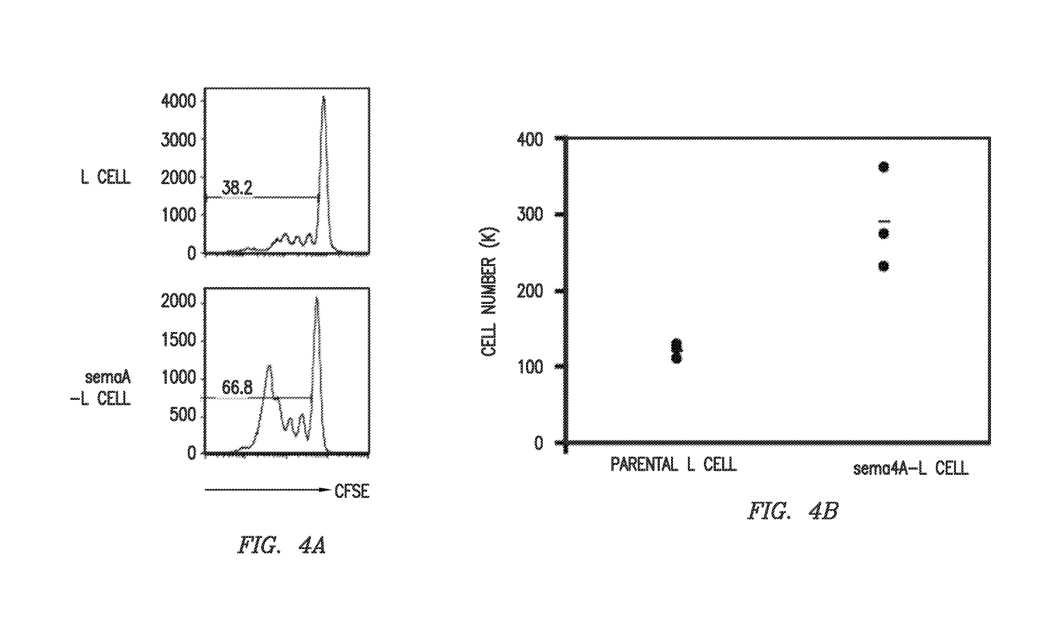

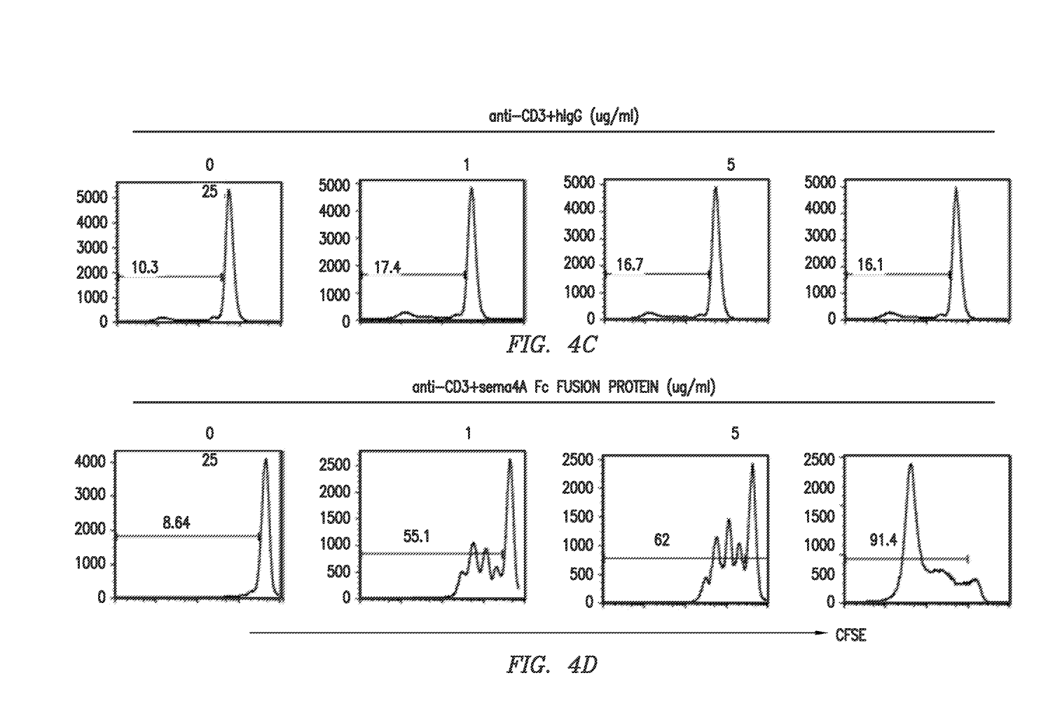

We established Sema4A transfected cell lines as APC. With this cell line, we cultured purified CD4+ naïve T cells in the presence of sub-optimal dose of OKT3 for 7 days, as it is shown in FIG. 4A CFSE dilution, and FIG. 4B Total cell number counting in three donors demonstrated that Sema4A transfected L cells could stimulate CD4+ naïve T cells expansion. Later, we detected if Sema4A-Ig fusion protein has the same function of stimulating CD4+ naïve T cell proliferation. As it is shown in FIGS. 4C & 4D, according to CFSE dilution, Sema4A-Ig fusion protein has synergistic effect on regulating CD4+ naïve T cells proliferation.

### Example V

### Human Anti-Sema4A Monoclonal Antibody Blocks Sema4A Mediated T Cell Proliferation in Presence of OKT3 and Anti-CD28

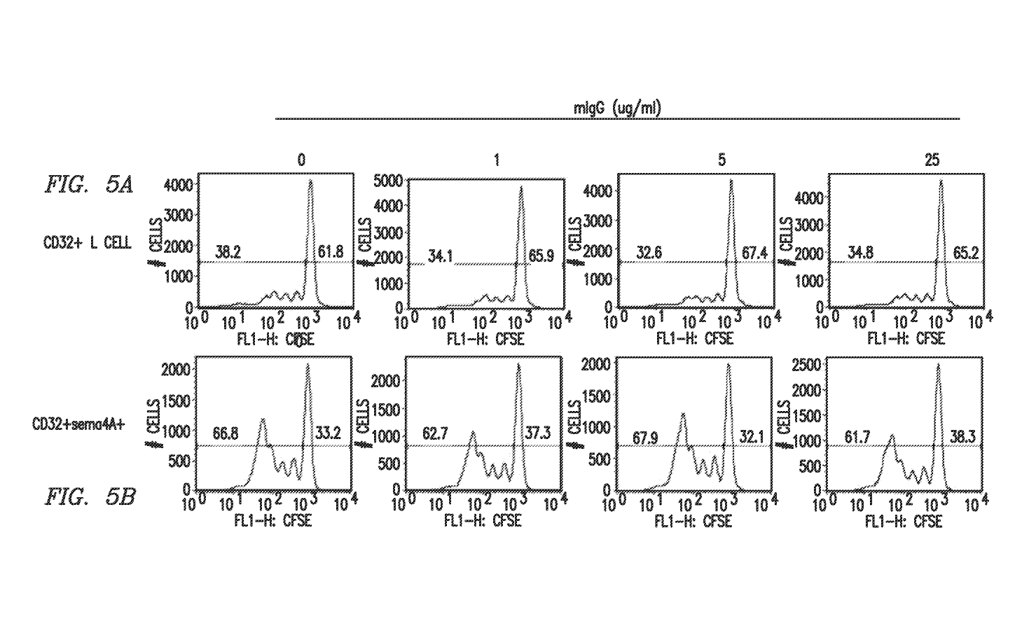

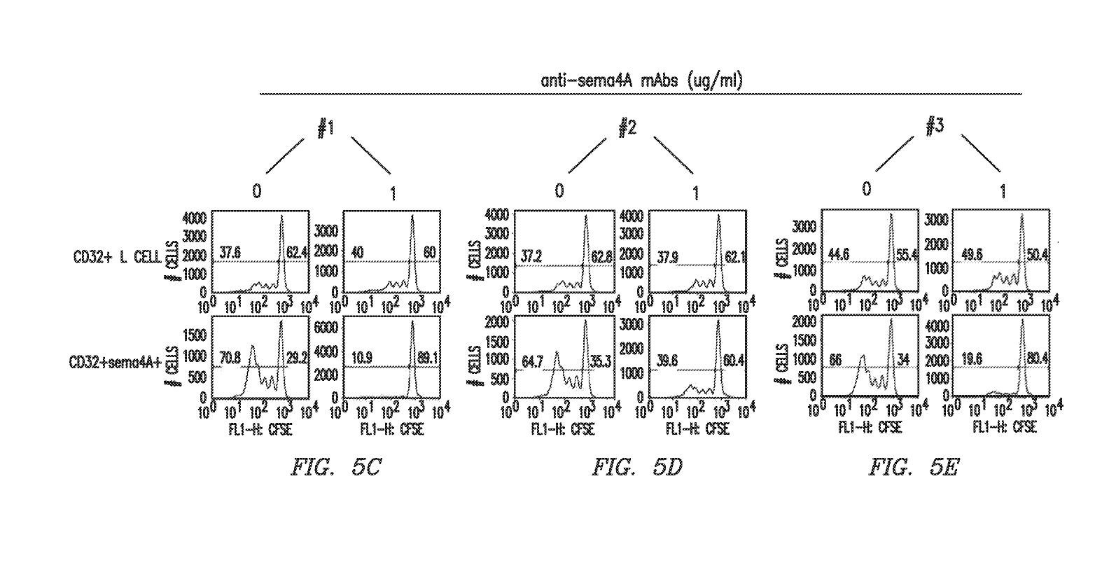

CD4+ naïve T cells were purified by cell sorting first, then labeled with CSFE, and then cultured with Sema4A transfected L cells or parental L cells under neutral condition (anti-CD3 and anti-CD28) in the presence of anti-Sema4A monoclonal antibody or mIgG for 7 days. As it is show in FIGS. 5A & 5B, according to CFSE dilution, T cell proliferation has been enhanced in Sema4A+L cell culture group comparing with parental L cell culture group.

As shown in FIGS. 5A & 5B, while mIgG could not prohibit T cell expansion stimulated by Sema4A. While three mAbs against Sema4A can suppress Sema4A mediated T cell proliferation in terms of Sema4A+ L cell culturing (FIGS. 5C, 5D & 5E

### Example VI

### Sema4A Promotes Naïve CD4+ T Cell Differentiation to TH2

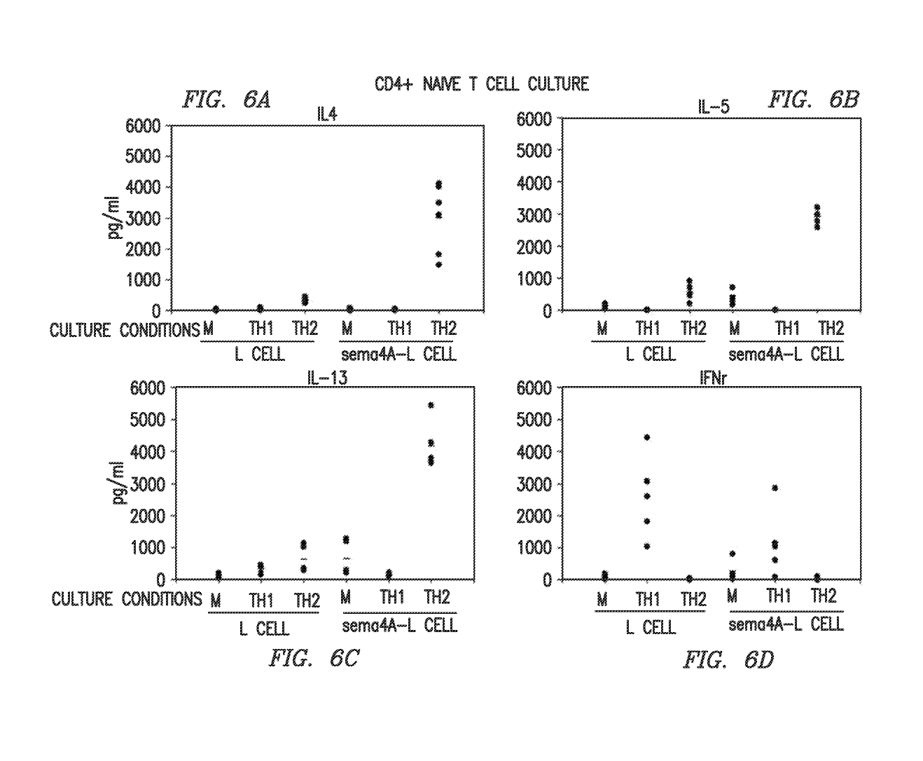

CD4+Tn cells isolated from human peripheral blood by cell sorting were cultured with human Sema4a expressing L cells or parental L cells in a natural condition (anti-CD3 plus anti-CD28), or Th1 condition (anti-CD3, anti-CD28, anti-IL-4 and IL-12), or Th2 condition (anti-CD3, anti-CD28, anti-IFNg, IL-4), respectively for 7 days. Cultured T cells were re-stimulated by anti-CD3 plus anti-CD28 for 24 hours and cytokines released into the culture supernatant were measured by ElSA (R&D systems). As shown in FIGS. 6A-D, while Th1 condition primed CD4+ naïve T cells produce high IFN-g, Th2 conditions primed naïve CD4+ T cells to produce Il-4, IL-5 and IL-13. Sema4A primed anti-CD3/anti-CD28 activated CD4+ naïve T cells to produce Th2 cytokines IL-13, IL-5 and to a lesser extend IL-4, but not much IFN-g. Most strikingly, Sema4A plus Th2 condition primed CD4+ naïve T cells to produced huge amounts of IL-4, IL-5 and IL-13, which were 4-5 times higher than that produced by Th2 cells induced by the conventional Th2 condition. Sema4A had no significant effect on Th1 cells induced by the Th1 condition.

### Example VII

### Sema4A Promotes TH2 Cytokine Production by CRTH2+ TH2 Memory T Cells Induced by IL-4

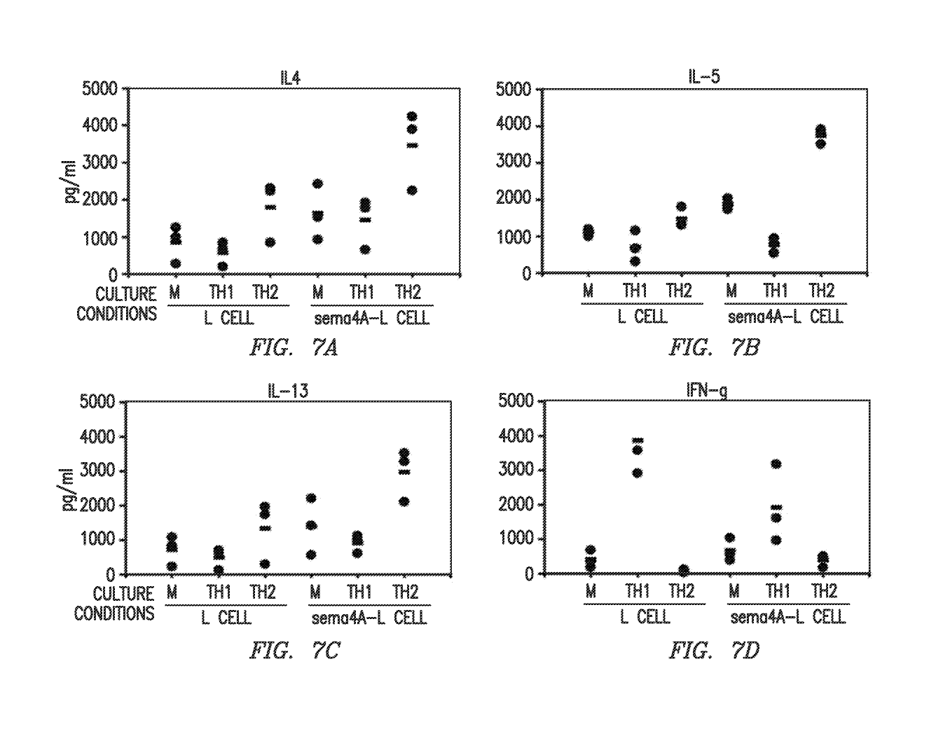

CRTH2+ memory T cells were purified by cell sorter first, then cultured with human Sema4a expressing L cells or parental L cells in a natural condition (anti-CD3 plus anti-CD28), or Th1 condition (anti-CD3, anti-CD28, anti-IL-4 and IL-12), or Th2 condition (anti-CD3, anti-CD28, anti-IFNg, IL-4), respectively for 7 days. Cultured T cells were restimulated by anti-CD3 plus anti-CD28 for 24 hours and cytokines released into the culture supernatant were measured by ElSA (R&D system). Sema4A alone promoted CRTH2+ TH2 memory T cells to produce TH2 cytokines, and Sema4A had strong synergistic effect with Th2 conditions in promoting CRTH2+ memory cells to produce Th2 cytokines (FIGS. 7A-7D). By contrast, Sema4A had no significant effect on CRTH2+ TH2 memory cells cultured in TH 1 condition.

### Example VIII

### Anti-Sema4A Monoclonal Ab Blocks Sema4A Mediated TH2 Cytokines Production

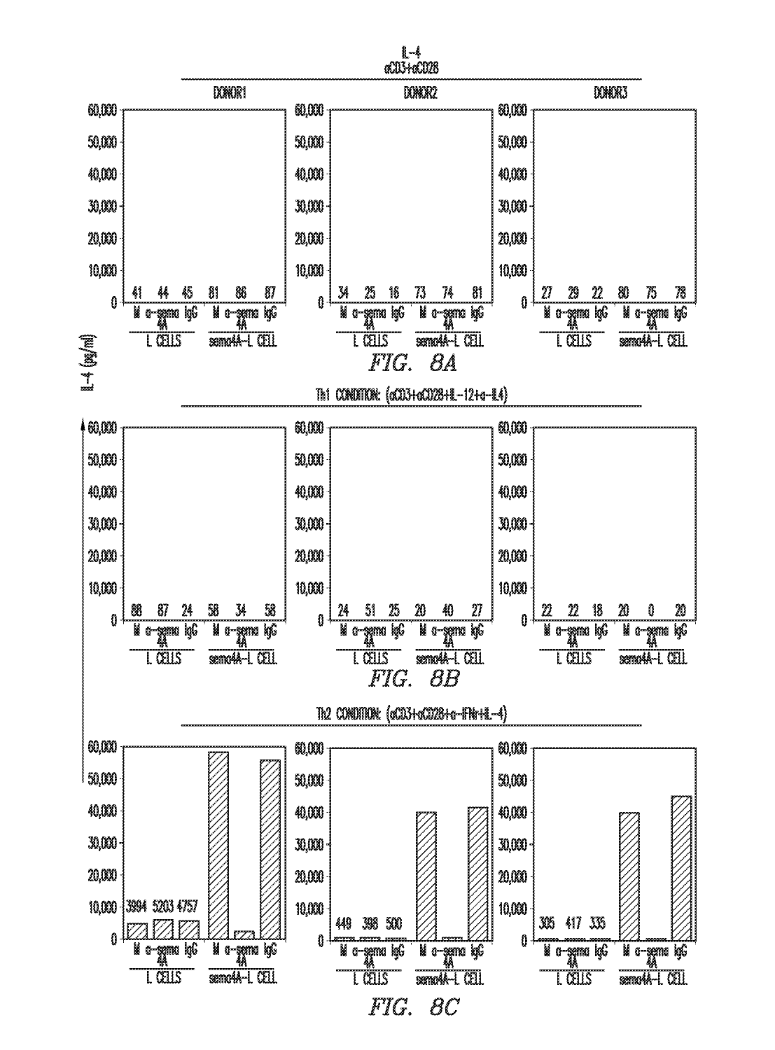

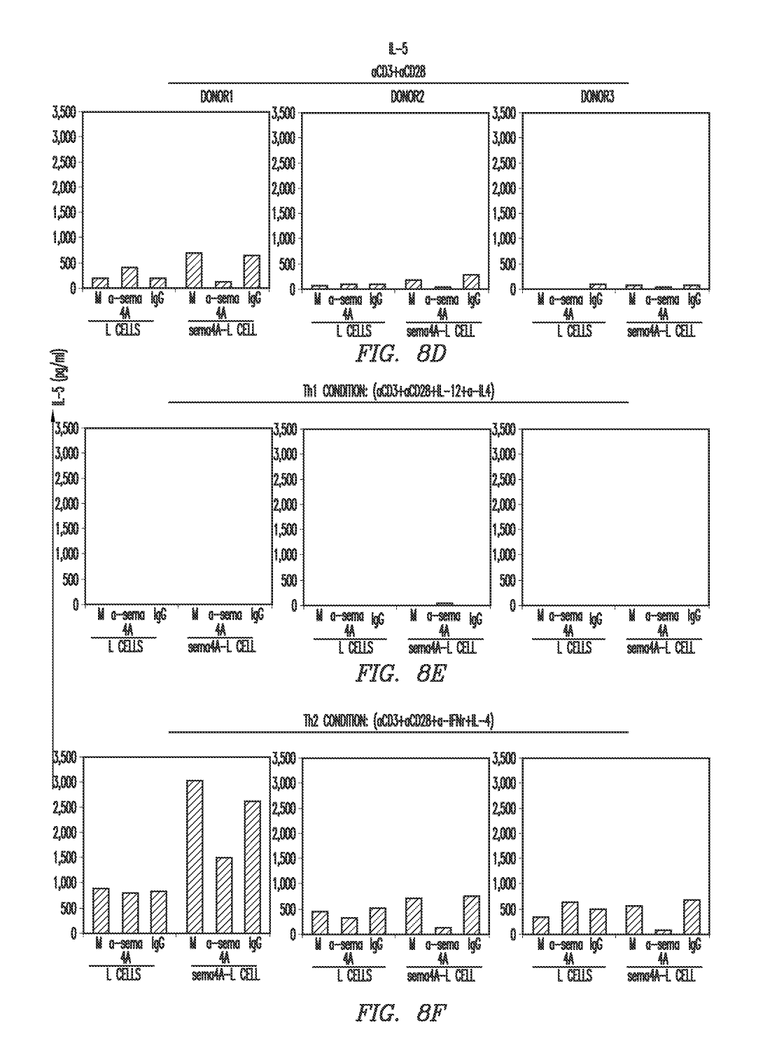

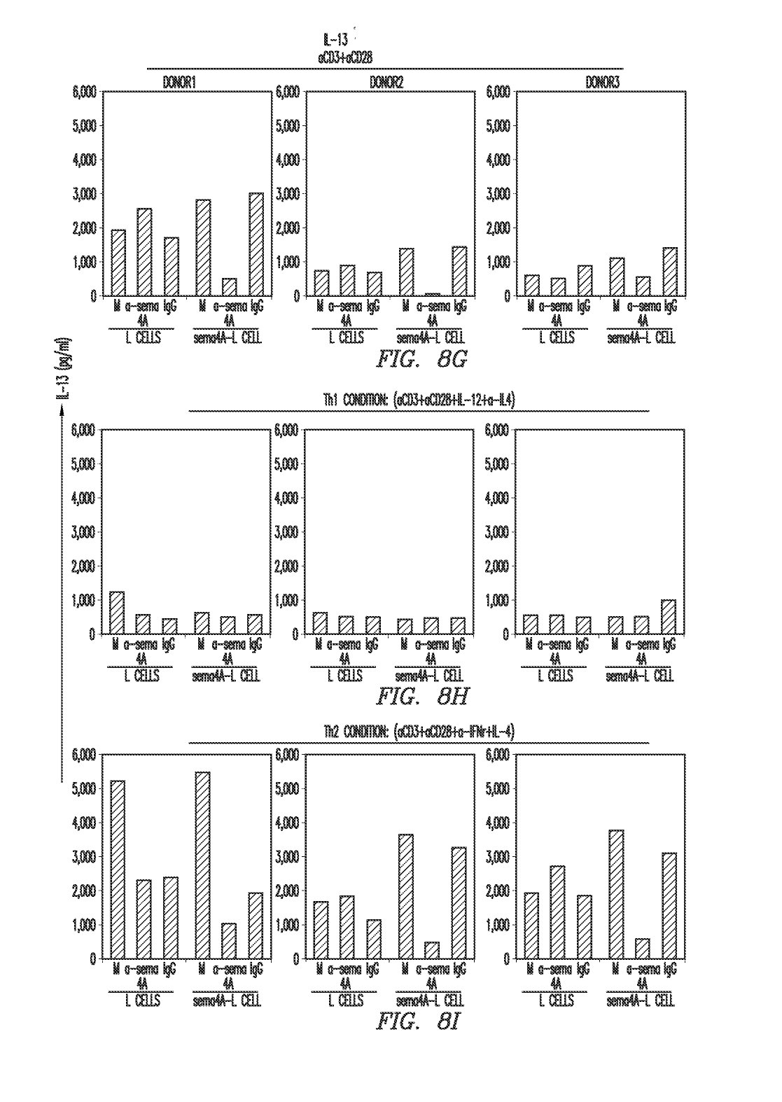

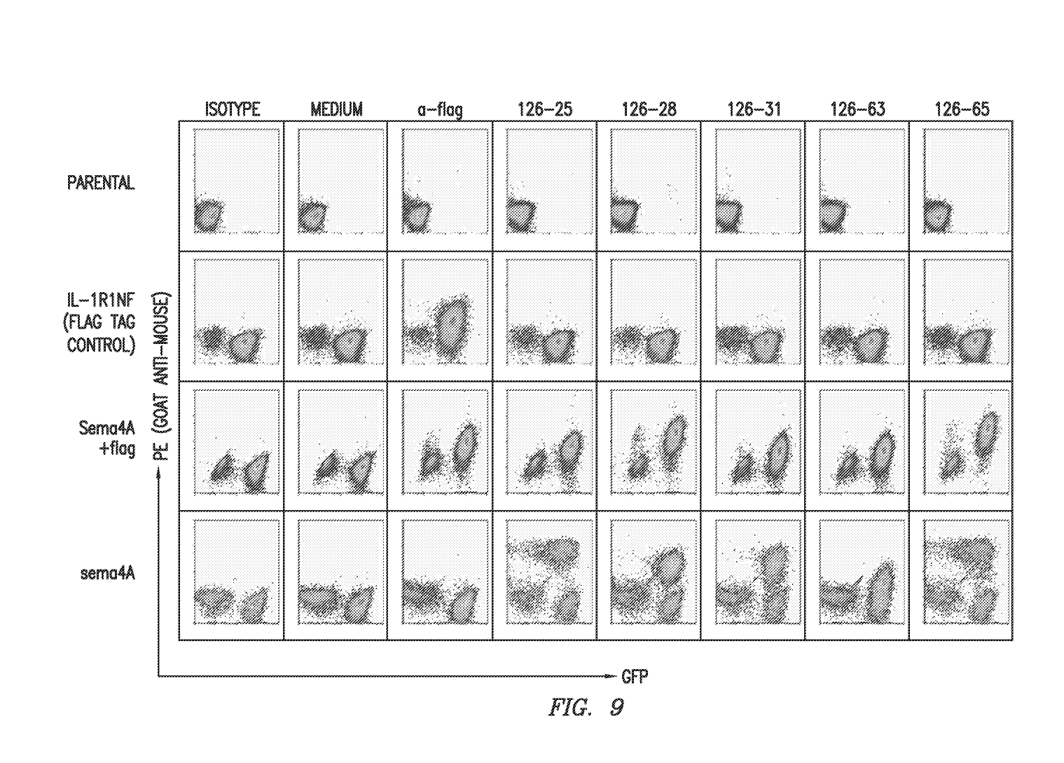

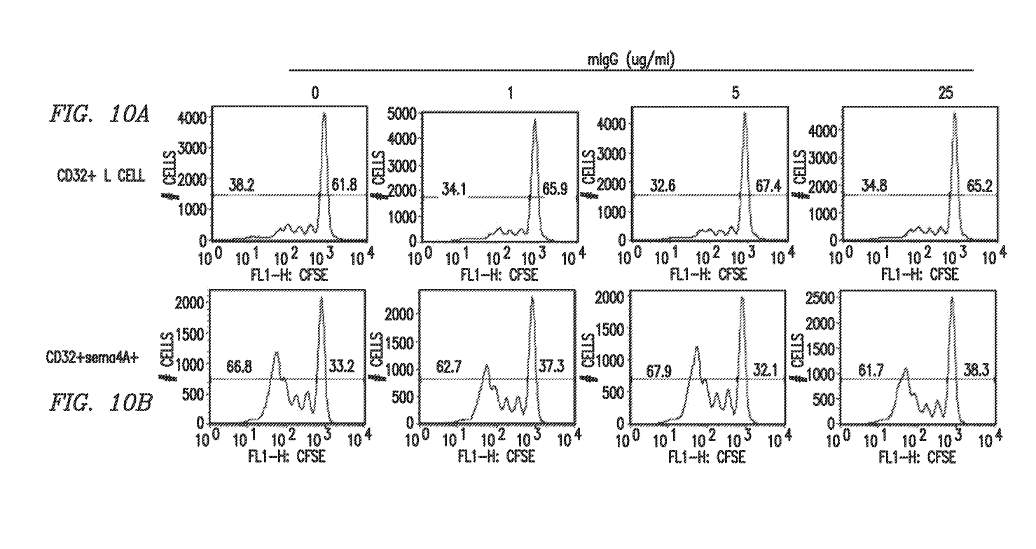

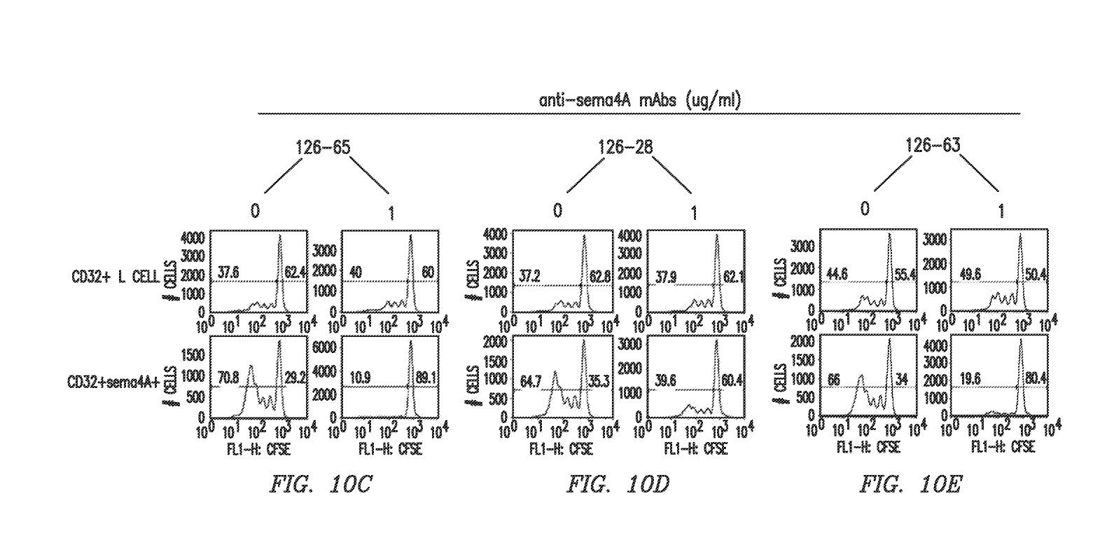

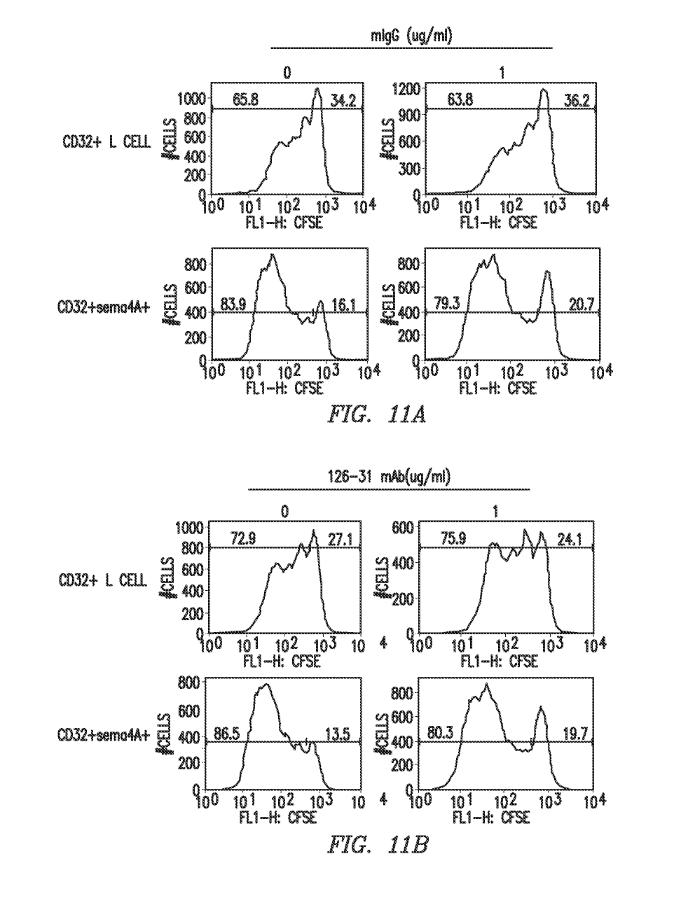

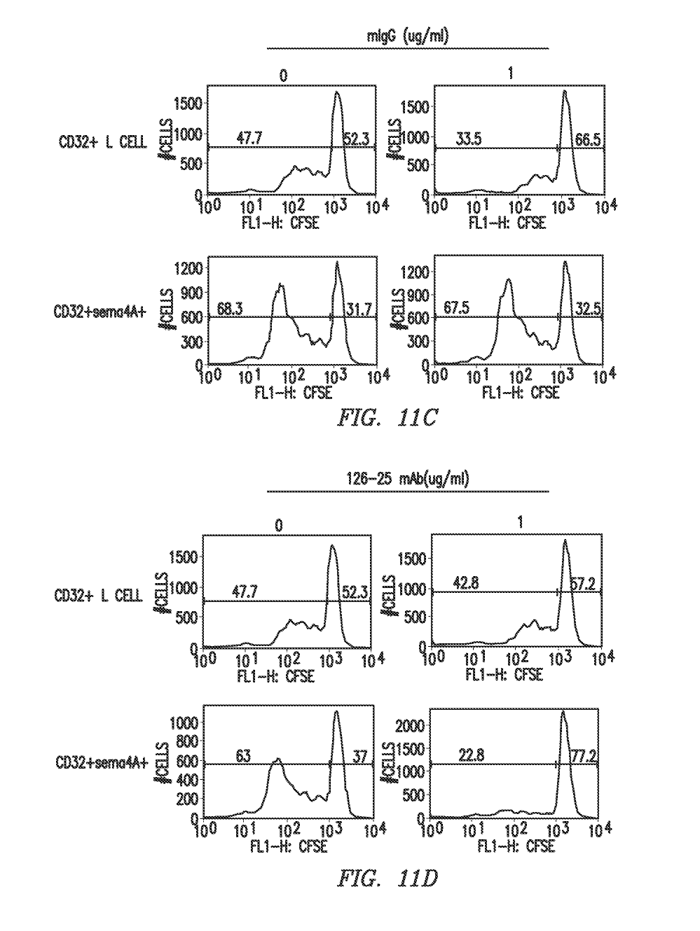

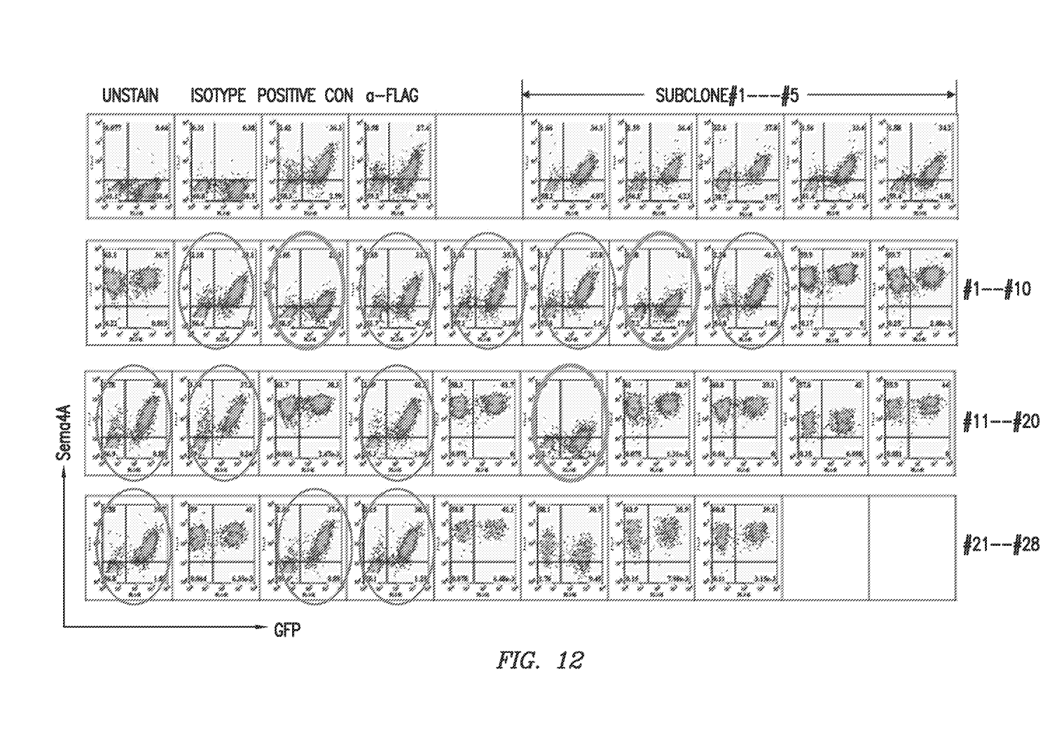

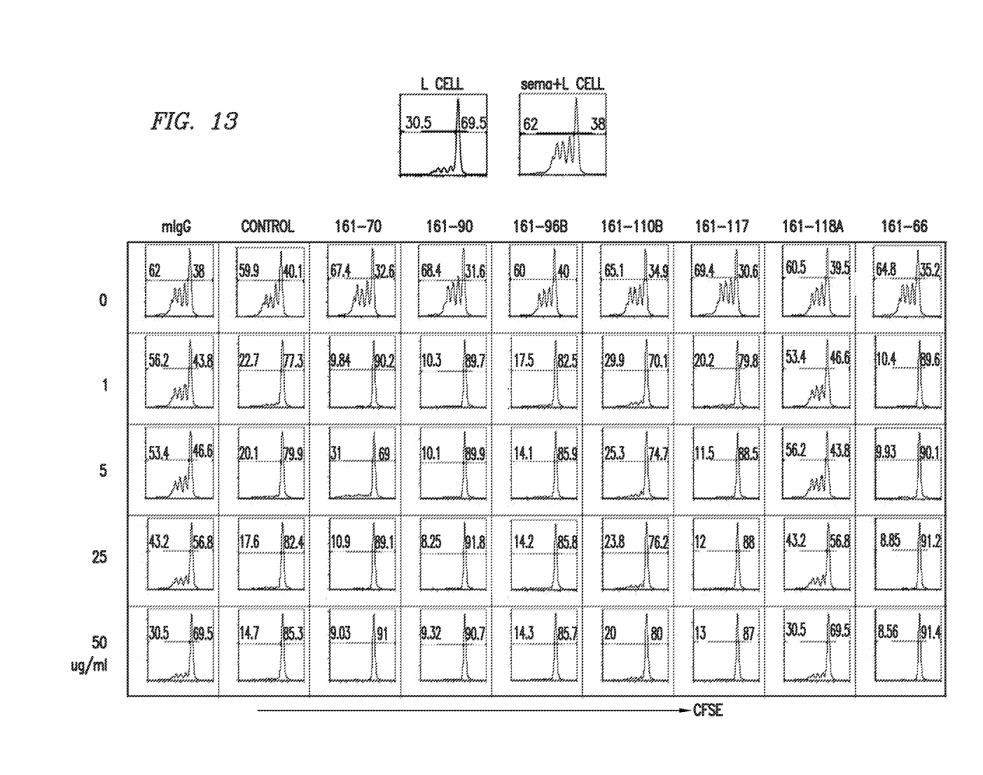

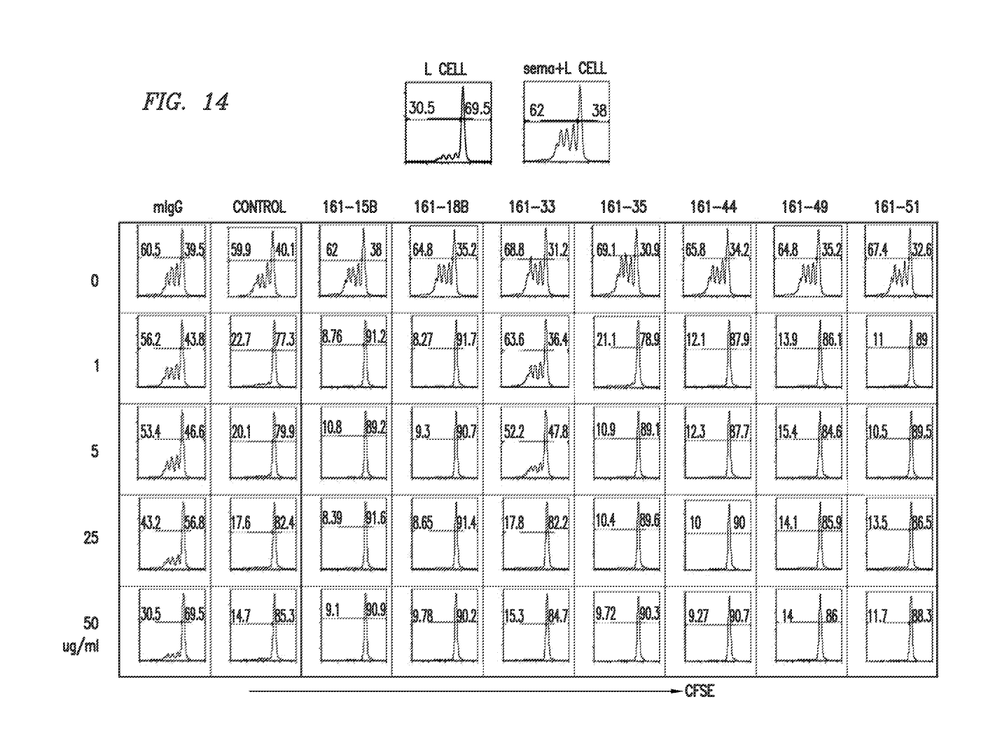

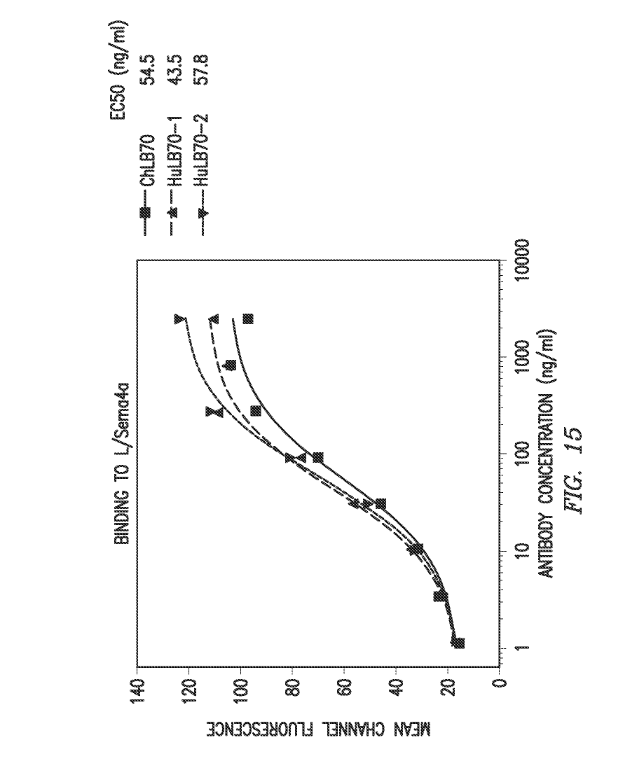

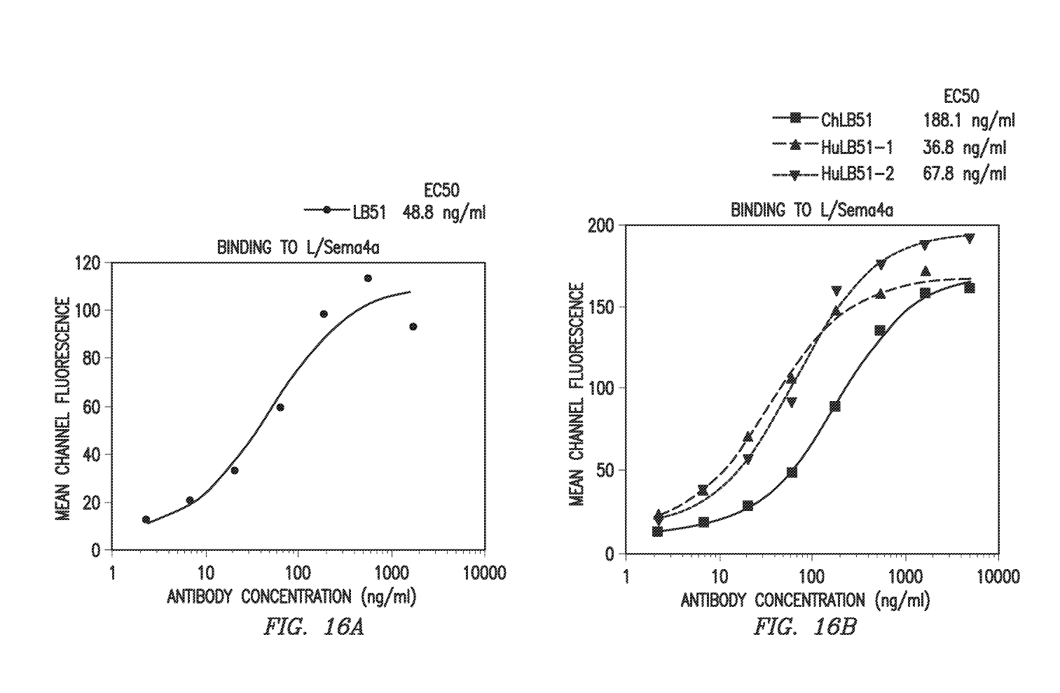

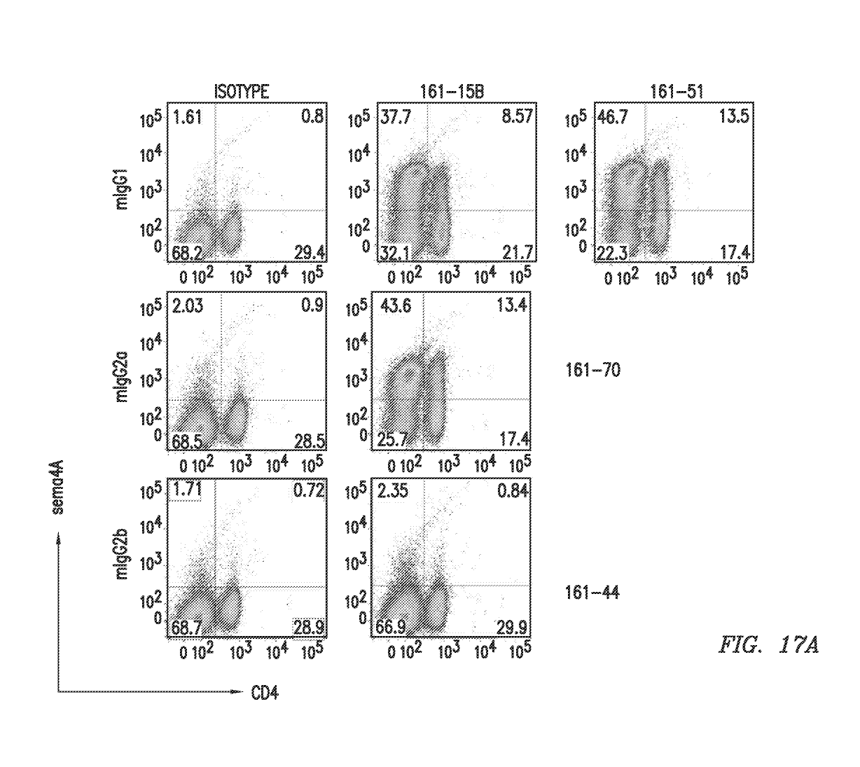

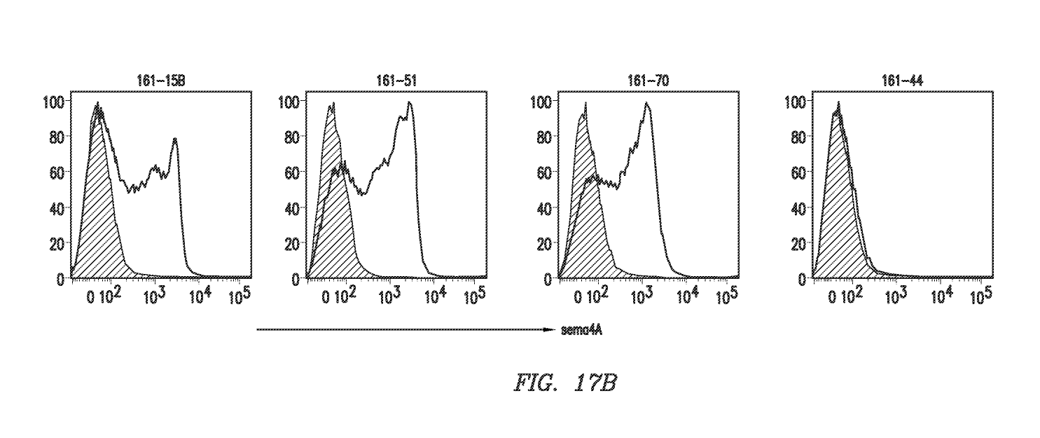

CD4+Tn cells isolated from human peripheral blood by cell sorting were cultured with human Sema4A expressing L cells or parental L cells under a natural condition (anti-CD3 plus anti-CD28), or Th1 condition (anti-CD3, anti-CD28, anti-IL-4 and IL-12), or Th2 condition (anti-CD3, anti-CD28, anti-IFNg, IL-4), with/without neutralizing mAb against human Sema4A respectively for 7 days. Cultured T cells were restimulated by anti-CD3 plus anti-CD28 for 24 hours and cytokines production were measured by EISA (R&D systems). FIGS. 8A through 81 shows that neutralizing mAb against Sema4A can block Th2 cytokines of IL-4 (FIG. 8A-C), IL-5 (FIGS. D-F) and IL-13 (FIGS. 8G-I) production which has been up-regulated by Sema4A in Th2 condition primed CD4+ naïve T cells in 3 donors.

### Example IX

### Functional Assay of Humanized mAbs Clone 161-51 and 161-70

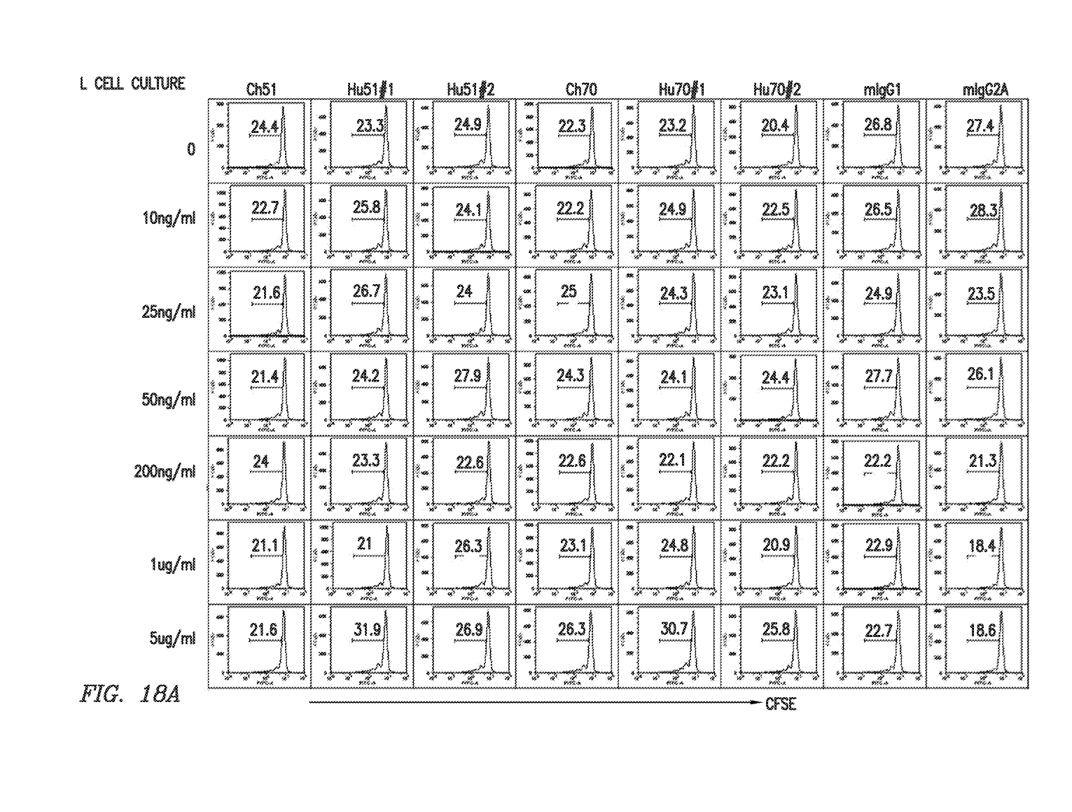

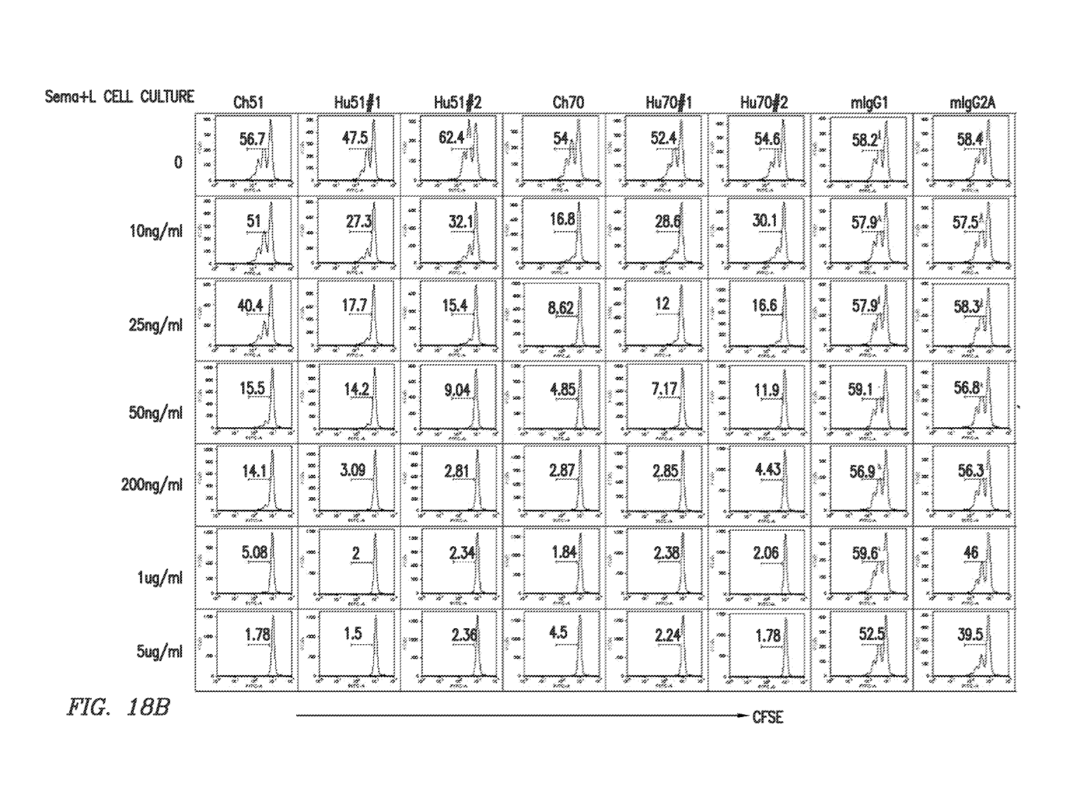

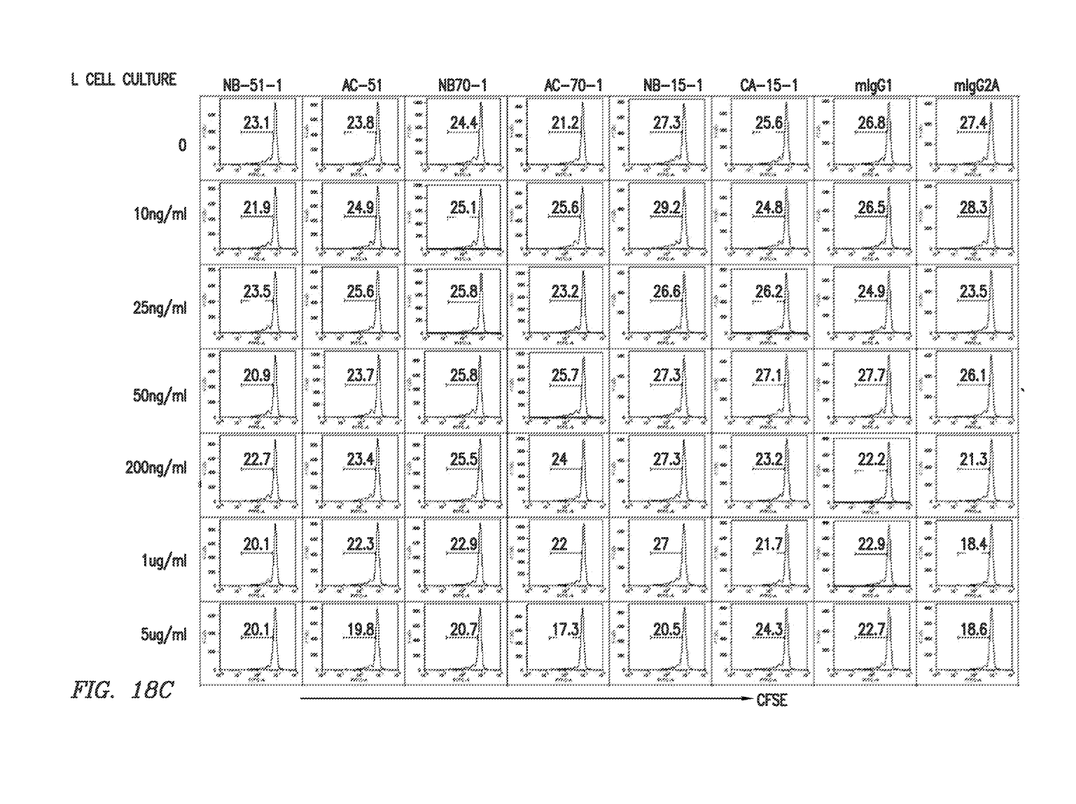

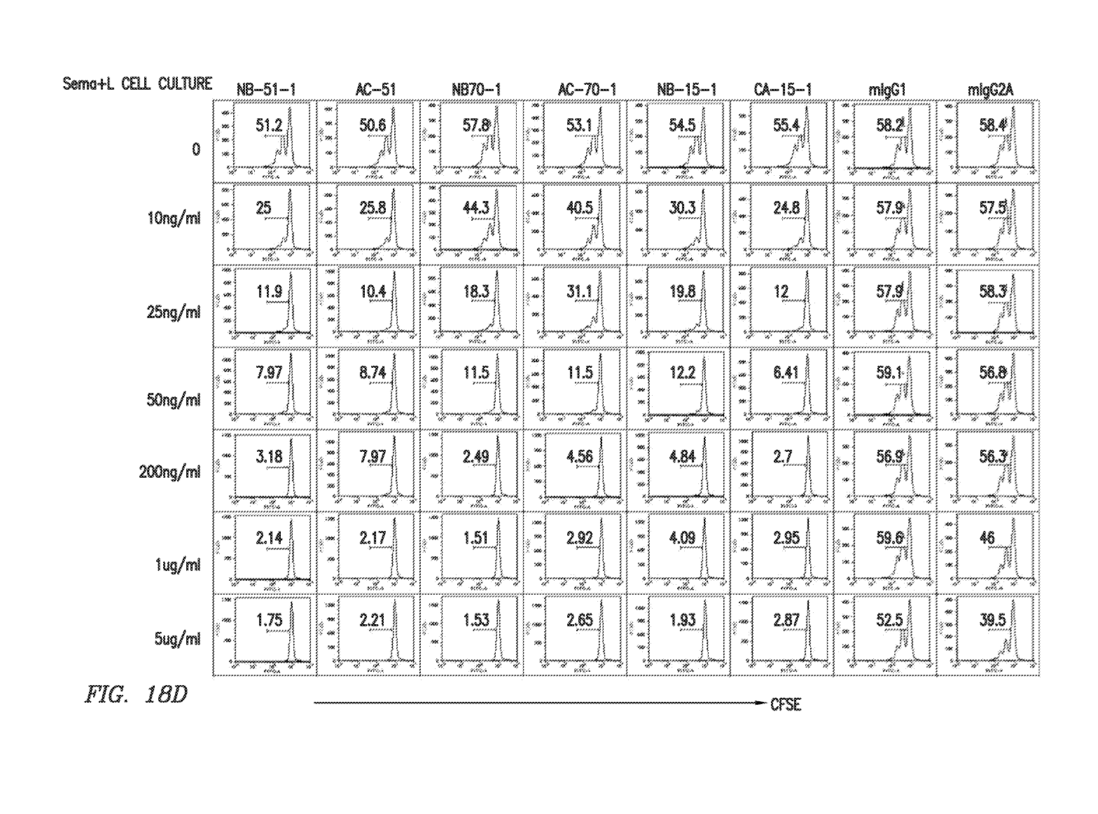

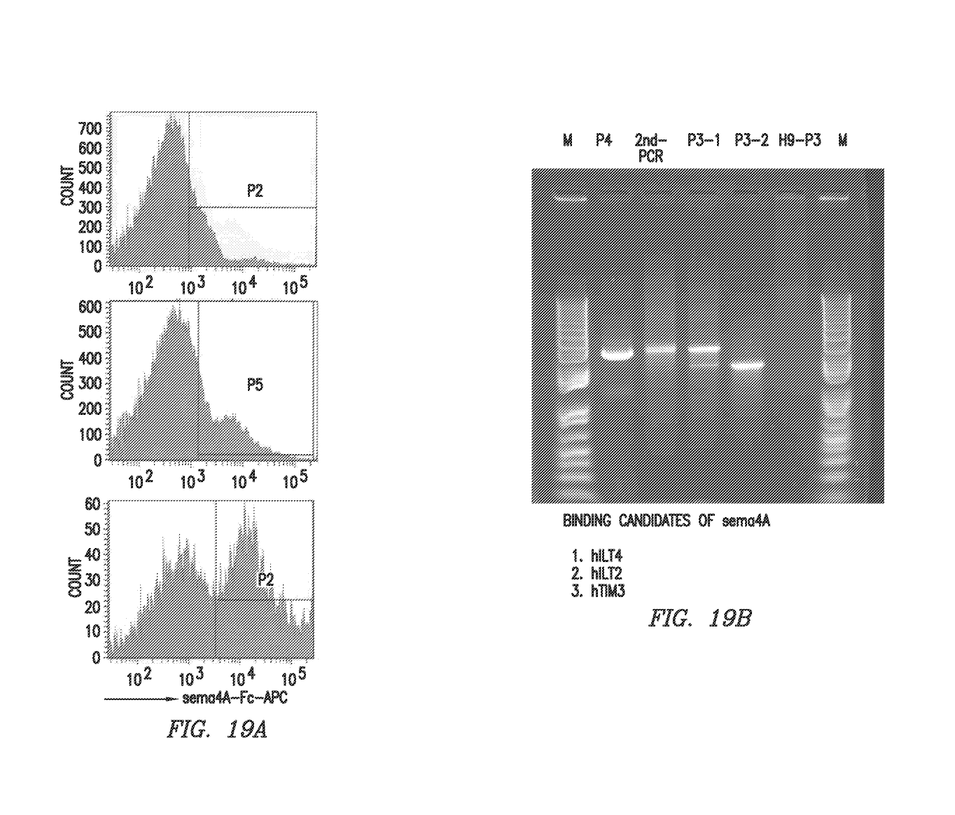

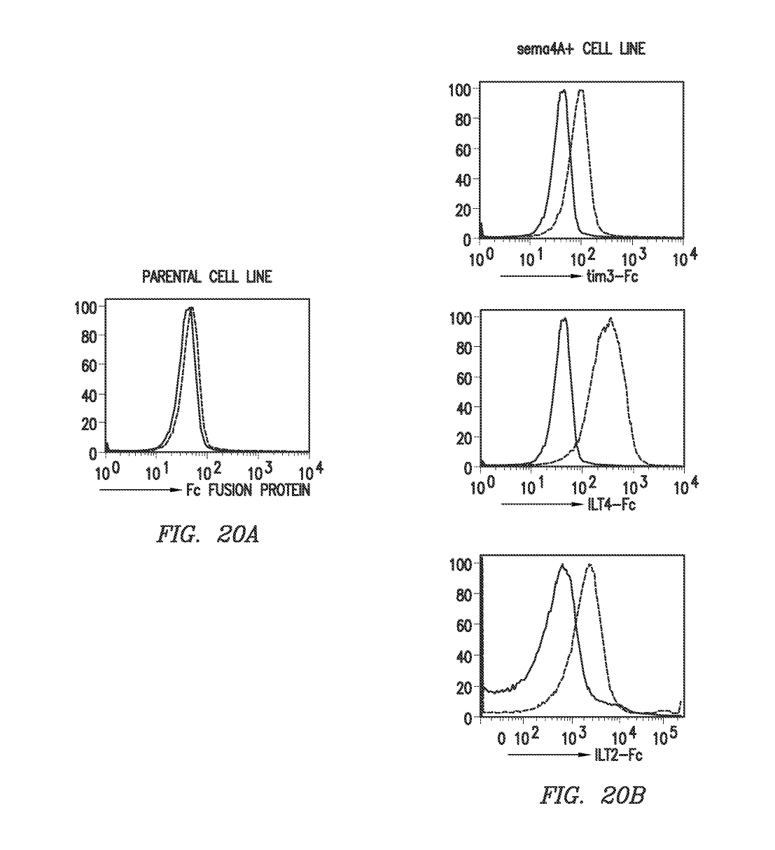

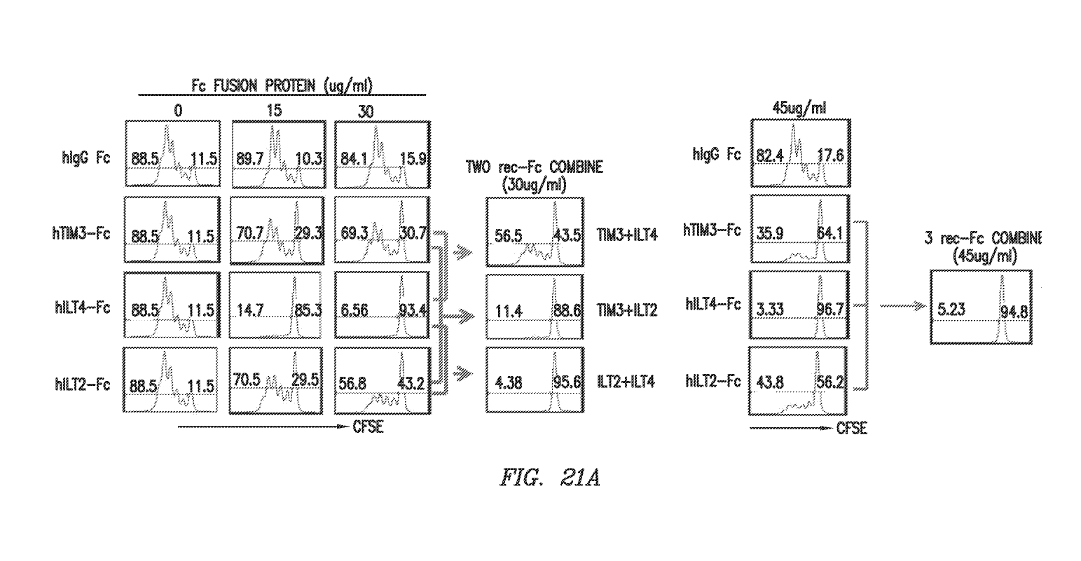

CD4+ naïve T cells were purified by cell sorting first, labeled with CSFE, then cultured with parental L cells or Sema4A transfected L cells under neutral conditions (anti-CD3 and anti-CD28) in the presence of humanized anti-hSema4A monoclonal antibody clone 161-51, 161-70 or mouse anti-hSema4A mAb clone 161-51, 161-70 or mIgG for 7 days. As it is shown in FIGS. 18A-D, according to CFSE dilution, T cell proliferation was enhanced in the Sema4A+L cell culture group as compared to the parental L cell culture group. Compared to mouse mAbs, humanized mAbs have very strong blocking function on suppressing T cell proliferation mediated by hSema4A.

### Example X

### Cloning the Sema4A Receptor with Expression cDNA Library Screening

CD4+ Tn cells were isolated by cell sorting first, then stimulated with immobilized OKT3 plus anti CD28 for 13 hrs until the putative receptors of Sema4A could be detected by FACS with Sema4A-Fc fusion protein staining. Total cDNA was isolated from pre-activated CD4+ T cells and expressing cDNA library was constructed by ATGC Company. The cDNA library expressing cell line was generated by transfecting cDNA library with retrovirus in target cells and enriched with Sema4A-Fc staining and sorting (FIG. 19A). The genome of enriched positive cells was isolated and applied as a template with specific primer design. Genomic PCR and cloning techniques were applied to amplify the inserted cDNA derived from cDNA library (FIG. 19B).

### Example XI

### Recombinant hILT4-Fc, hILT2-Fc and hTIM3-FC Block Sema4A Mediated CD4+ T Cell Proliferation

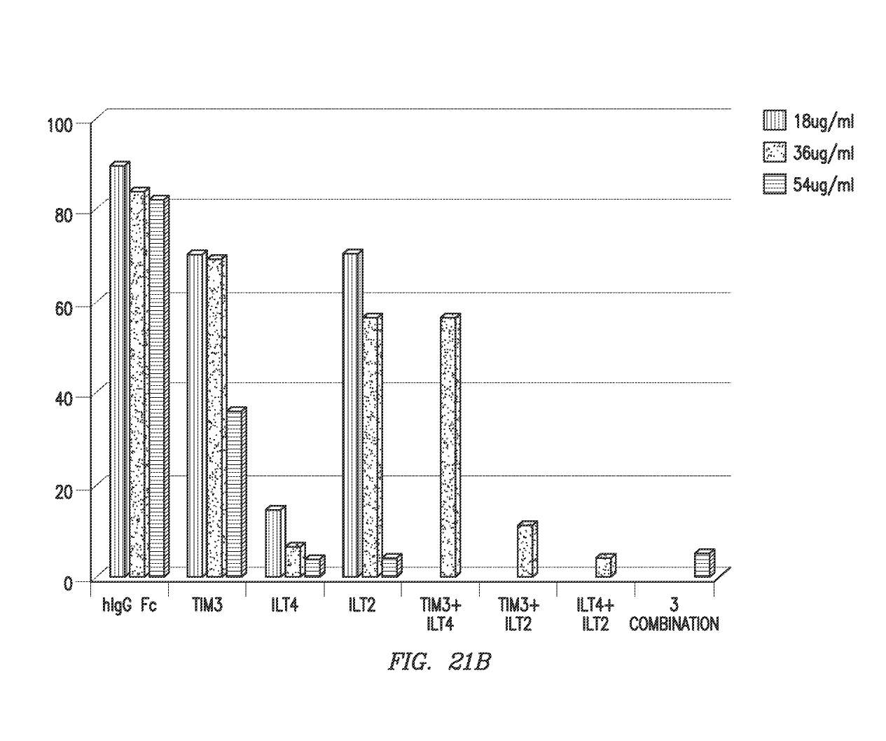

Purified CD4+ naïve T cells were labeled with CFSE, then cultured with sub-optimal dose of OKT3 plus hSema4A-Fc in presence of recombinant hILT4-Fc, hILT2-Fc and hTIM3-FC fusion protein individually or combined in a dose dependent manner for 7 days (hIgG Fe as control group). T cell proliferation was detected by CFSE dilution. The numbers in each square represents the percentage of proliferated T cells as shown in FIG. 21A. FIG. 21B shows that receptors-Fc has synergistic effects on suppressing CD4+ T cell proliferation mediated by hSema4A through CFSE dilution.

### Example XII

### Sema4A is Over-Expressed in Asthma Lung Tissue

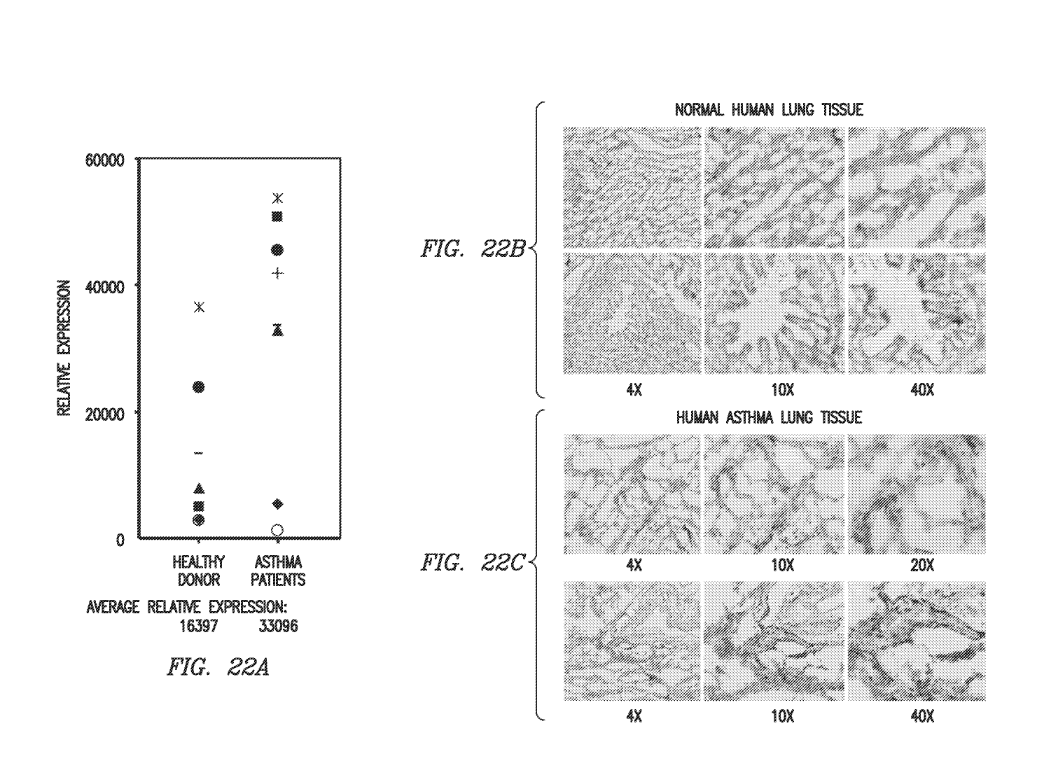

Relative expression of hsema4A in human Lung tissue at RNA level was tested by Q-PCR analysis. Total RNA of lung tissues from asthma or healthy donor tissue was isolated and reverse transcription of the RNA into cDNAs was used as template, with hsema4A (“human sema4A”) as the specific primer. Q-PCR analysis was applied. The expression of hsema4A was evaluated as shown in FIG. 22A. Each dot represents one donor and the short line in each group represents the average expression.

Immune-histologic test of hSema4A expression in human frozen sections was prepared. As shown in FIG. 22B, Sema4A cannot be detected in human normal lung tissue. As further shown by the data in FIG. 22C, Sema4A is highly and specifically expressed on the infiltrated cells in the human asthma lung tissue.

1. The isolated antibody of claim 9, comprising: (a) a heavy chain variable region CDR1 comprising an amino acid sequence at least 90% identical to the amino acid sequence of SEQ ID NO: 36; (b) a heavy chain variable region CDR2 comprising an amino acid sequence at least 90% identical to the amino acid sequence of SEQ ID NO: 37; (c) a heavy chain variable region CDR3 comprising an amino acid sequence at least 90% identical to the amino acid sequence of SEQ ID NO. 38; (d) a light chain variable region CDR1 comprising an amino acid sequence at least 90% identical to the amino acid sequence of SEQ ID NO. 48; (e) a light chain variable region CDR2 comprising an amino acid sequence at least 90% identical to the amino acid sequence of SEQ ID NO. 49; and (f) a light chain variable region CDR3 comprising an amino acid sequence at least 90% identical to the amino acid sequence of SEQ ID NO. 50.

2. The isolated antibody of claim 9, comprising: (a) a heavy chain variable region CDR1 comprising an amino acid sequence at least 90% identical to the amino acid sequence of SEQ ID NO: 54; (b) a heavy chain variable region CDR2 comprising an amino acid sequence at least 90% identical to the amino acid sequence of SEQ ID NO: 55; (c) a heavy chain variable region CDR3 comprising an amino acid sequence at least 90% identical to the amino acid sequence of SEQ ID NO. 56; (d) a light chain variable region CDR1 comprising an amino acid sequence at least 90% identical to the amino acid sequence of SEQ ID NO. 66; (e) a light chain variable region CDR2 comprising an amino acid sequence at least 90% identical to the amino acid sequence of SEQ ID NO. 67; and (f) a light chain variable region CDR3 comprising an amino acid sequence at least 90% identical to the amino acid sequence of SEQ ID NO. 68.

3-8. (canceled)

9. An isolated antibody that binds to an epitope on human sema4A recognized by an antibody having a heavy chain variable region comprising the amino acid sequences of SEQ ID NO. 39, 45, 57, or 63 and a light chain variable region comprising the amino acid sequences of SEQ ID No. 51 or 69.

10-13. (canceled)

14. A method of treating autoimmune disease in a patient in need thereof comprising the step of administering a therapeutically effective amount of an anti-human sema4A antibody to said patient wherein T cell proliferation and/or Th2 differentiation is blocked.

15. A method of treating cancer in a patient in need thereof comprising the step of administering a therapeutically effective amount of an anti-human sema4A antibody to said patient wherein T cell proliferation and/or Th2 differentiation is blocked.

16. A method of treating allergic disease in a patient in need thereof comprising the step of administering a therapeutically effective amount of an anti-human sema4A antibody to said patient wherein T cell proliferation and/or Th2 differentiation is blocked.

17. The isolated antibody of claim 1, wherein the antibody comprises (a) a heavy chain variable region CDR1 comprising the amino acid sequence of SEQ ID NO: 36; (b) a heavy chain variable region CDR2 comprising the amino acid sequence of SEQ ID NO: 37; (c) a heavy chain variable region CDR3 comprising the amino acid sequence of SEQ ID NO. 38; (d) a light chain variable region CDR1 comprising the amino acid sequence of SEQ ID NO. 48; (e) a light chain variable region CDR2 comprising the amino acid sequence of SEQ ID NO. 49; and (f) a light chain variable region CDR3 comprising the amino acid sequence of SEQ ID NO. 50.

18. The isolated antibody of claim 2, wherein the antibody comprises (a) a heavy chain variable region CDR1 comprising the amino acid sequence of SEQ ID NO: 54; (b) a heavy chain variable region CDR2 comprising the amino acid sequence of SEQ ID NO: 55; (c) a heavy chain variable region CDR3 comprising the amino acid sequence of SEQ ID NO. 56; (d) a light chain variable region CDR1 comprising the amino acid sequence of SEQ ID NO. 66; (e) a light chain variable region CDR2 comprising the amino acid sequence of SEQ ID NO. 67; and (f) a light chain variable region CDR3 comprising the amino acid sequence of SEQ ID NO. 68.

19. the antibody of claim 9, wherein the antibody is a humanized antibody.

20. A pharmaceutical composition comprising, an isolated antibody according to claim 9 in a pharmaceutically acceptable carrier.

21. The method of claim 14, wherein the anti-human sema4A antibody in an antibody in accordance with claim 9.

22. The method of claim 15, wherein the anti-human sema4A antibody in an antibody in accordance with claim 9.

23. The method of claim 16, wherein the anti-human sema4A antibody in an antibody in accordance with claim 9.


SEQUENCE LISTING (77 sequences)

SEQ ID NO: 1 - protein, 5 aa, Mus sp.
SHTMH

SEQ ID NO: 2 - protein, 17 aa, Mus sp.
YINPSSGYTNYNQKFKD

SEQ ID NO: 3 - protein, 8 aa, Mus sp.
WDYVALDY

SEQ ID NO: 4 - protein, 136 aa, Mus sp.
MERHWIFLFLLSVTAGVHSQVQLQQSGAELARPGASVKMSCKASGYTFTSHTMHWVKQRPGQGLEWIGYINPSSGYTNYNQKFKDKATLTADKSSSTAYMQLSSLTSEDSAVYYCARWDYVALDYWGQGTSVTVSS

SEQ ID NO: 5 - DNA, 408 bp, Mus sp.
atggaaaggcactggatctttctcttcctgttgtcagtaactgcaggtgtccactcccaggtccagctgcagcagtctggggctgaactggcaagacctggggcctcagtgaagatgtcctgcaaggcttctggctacacctttactagccacacgatgcactgggtaaaacagaggcctggacagggtctggaatggattggatacattaatcctagcagtggttatactaattacaatcagaagttcaaggacaaggccacattgactgcagacaaatcctccagcacagcctacatgcaactgagcagcctgacatctgaggactctgcagtctattactgtgcaagatgggattacgttgctctggactactggggtcaaggaacctcagtcaccgtctcctca

SEQ ID NO: 6 - protein, 11 aa, Mus sp.
SASQGISNYLN

SEQ ID NO: 7 - protein, 7 aa, Mus sp.
YISSLHS

SEQ ID NO: 8 - protein, 9 aa, Mus sp.
QQYSKLPYT

SEQ ID NO: 9 - protein, 127 aa, Mus sp.
MMSSAQFLGLLLLCFQGTRCEIQMTQTTSSLSASLGDRVTISCSASQGISNYLNWYQQKPDGTVKLLIYYISSLHSGVTSRFSGSGSGTDYSLTISNLEPEDIATYYCQQYSKLPYTFGGGTKLEIK

SEQ ID NO: 10 - DNA, 381 bp, Mus sp.
atgatgtcctctgctcagttccttggtctcctgttgctctgttttcaaggtaccagatgtgagatccagatgacacagactacatcctccctgtctgcctctctgggagacagagtcaccatcagttgcagtgcaagtcagggcattagcaattatttaaactggtatcagcagaaaccagatggaactgttaaactcctgatctattacatatcaagtttacactcaggagtcacatcaaggttcagtggcagtgggtctgggacagattattctctcaccatcagcaacctggaacctgaagatattgccacttactattgtcagcagtatagtaagcttccgtacacgttcggaggggggaccaagctggaaataaaa

SEQ ID NO: 11 - protein, 5 aa, Mus sp.
DYNMD

SEQ ID NO: 12 - protein, 17 aa, Mus sp.
DINPKYDSTTYNQKFKG

SEQ ID NO: 13 - protein, 16 aa, Mus sp.
ERGSYDYDGTPYAMDY

SEQ ID NO: 14 - protein, 144 aa, Mus sp.
MEWSWIFLFLLSGTAGVLSEVQLQQFGAELVKPGASVKISCKASGYTFTDYNMDWVKQSHGKSLEWIGDINPKYDSTTYNQKFKGKATLTADKSSSTAYMELRSLTSEDTAVYYCARERGSYDYDGTPYAMDYWGQGTSVTVSS

SEQ ID NO: 15 - DNA, 432 bp, Mus sp.
atggaatggagctggatctttctctttctcctgtcaggaactgcaggtgtcctctctgaggtccagctgcaacagtttggagctgagctggtgaagcctggggcttcagtgaagatatcctgcaaggcttctggctacacattcactgactacaacatggactgggtgaagcagagccatggaaagagccttgagtggattggagatattaatcctaagtatgatagtactacctacaaccagaagttcaagggaaaggccacattgaccgcagacaagtcctccagcacagcctacatggagctccgcagcctgacatctgaggacactgcagtctattactgtgcaagagagaggggctcctatgattacgacgggactccctatgctatggactactggggtcaaggaacctcagtcaccgtctcctca

SEQ ID NO: 16 - protein, 17 aa, Mus sp.
KSSQSLLYSSNRKNYLA

SEQ ID NO: 17 - protein, 7 aa, Mus sp.
WASTRES

SEQ ID NO: 18 - protein, 9 aa, Mus sp.
QQYYTYPRT

SEQ ID NO: 19 - protein, 133 aa, Mus sp.
MDSQAQVLMLLLLWVSGTCGDIVMSQSPSSLAVSVGEKVTMSCKSSQSLLYSSNRKNYLAWYQQKPGQSPKLLIYWASTRESGVPDRFTGSGSGTDFTLTISSVKAEDLAVYYCQQYYTYPRTFGGGTKLEIK

SEQ ID NO: 20 - DNA, 399 bp, Mus sp.
atggattcacaggcccaggttcttatgttactgctgctatgggtatctggtacctgtggggacattgtgatgtcacagtctccatcctccctagctgtgtcagttggagagaaggttactatgagctgcaagtccagtcagagccttttatatagtagcaatcgaaagaactacttggcctggtaccagcagaaaccagggcagtctcctaaactgctgatttactgggcgtccactagggaatctggggtccctgatcgcttcacaggcagtggatctgggacagatttcactctcaccatcagcagtgtgaaggctgaagacctggcagtttattactgtcagcaatattatacctatcctcggacgttcggtggaggcaccaagctggaaatcaaa

SEQ ID NO: 21 - protein, 5 aa, Mus sp.
SYWVN

SEQ ID NO: 22 - protein, 17 aa, Mus sp.
EINPSNGRTDYNEKFKS

SEQ ID NO: 23 - protein, 7 aa, Mus sp.
GLDYFDY

SEQ ID NO: 24 - protein, 135 aa, Mus sp.
MGWSYIILFLVATATDVHSQVQLQQPGAELVKPGASVKLSCKASGYTFTSYWVNWVKQRPGQGLEWIGEINPSNGRTDYNEKFKSKAIMIVDKSSSTAYMQFSSLTSEDSAVYYCAGGLDYFDYWGQGTTLTVSS

SEQ ID NO: 25 - DNA, 405 bp, Mus sp.
atgggatggagctatatcatcctctttttggtagcaacagctacagatgtccactcccaggtccaactgcagcagcctggggctgaactggtgaagcctggggcttcagtgaagctgtcctgcaaggcttctggctacaccttcaccagctactgggtgaactgggtgaagcagaggcctggacaaggccttgagtggattggagagattaatcctagcaacggtcgtactgactacaatgagaagttcaagagcaaggccataatgattgtagacaaatcttctagcacagcctatatgcaattcagcagcctgacatctgaggactctgcggtctattactgtgcagggggactggactactttgactattggggccaaggcaccactctcacagtctcctca

SEQ ID NO: 26 - protein, 11 aa, Mus sp.
KASENVGTYVS

SEQ ID NO: 27 - protein, 7 aa, Mus sp.
GASNRYT

SEQ ID NO: 28 - protein, 9 aa, Mus sp.
GQSYSYPFT

SEQ ID NO: 29 - protein, 127 aa, Mus sp.
MESQTLVFISILLWLYGADGNIVMTQSPKSMSMSVGERVTLSCKASENVGTYVSWYQQKPDQSPKLLIYGASNRYTGVPDRFTGSGSATDFTLTISSVQAEDLADYHCGQSYSYPFTFGSGTSLEIK

SEQ ID NO: 30 - DNA, 381 bp, Mus sp.
atggaatcacagactctggtcttcatatccatactgctctggttatatggtgctgatgggaacattgtaatgacccaatctcccaaatccatgtccatgtcagtaggagagagggtcaccttgagctgcaaggccagtgagaatgtgggtacttatgtatcctggtatcaacagaaaccagaccagtctcctaaactgctgatatacggggcatccaaccggtacactggggtccccgatcgcttcacaggcagtggatctgcaacagatttcactctgaccatcagcagtgtgcaggctgaagaccttgcagattatcactgtggacagagttacagctatccattcacgttcggctcggggacaagtttggaaataaaa

SEQ ID NO: 31 - protein, 16 aa, Mus sp.
KSSQSLLDRDGKTYLN

SEQ ID NO: 32 - protein, 7 aa, Mus sp.
LVSKLDS

SEQ ID NO: 33 - protein, 9 aa, Mus sp.
CQGTHFPFT

SEQ ID NO: 34 - protein, 132 aa, Mus sp.
MMSPAQFLFLLVLWIREANGAVVMTQTPLTLSVTIGQPASISCKSSQSLLDRDGKTYLNWLLQRPGQSPKRLIYLVSKLDSGVPDRFTGSGSGTDFTLKISRVEAEDLGVYYCCQGTHFPFTFGSGTKLEIK

SEQ ID NO: 35 - DNA, 396 bp, Mus sp.
atgatgagtcctgcccagttcctgtttctgttagtgctctggattcgggaagccaacggtgctgttgtgatgacccagactccactcactttgtcggttaccattggacaaccagcctccatctcttgcaagtcaagtcagagcctcttagatcgtgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctgtcaaggtacacattttccattcacgttcggctcggggacaaagttggaaataaaa

SEQ ID NO: 36 - protein, 5 aa, Artificial Sequence
  Description of Artificial Sequence Synthetic peptide
SHTMH

SEQ ID NO: 37 - protein, 17 aa, Artificial Sequence
  Description of Artificial Sequence Synthetic peptide
YINPSSGYTNYNQKFKD

SEQ ID NO: 38 - protein, 8 aa, Artificial Sequence
  Description of Artificial Sequence Synthetic peptide
WDYVALDY

SEQ ID NO: 39 - protein, 117 aa, Artificial Sequence
  Description of Artificial Sequence Synthetic polypeptide
QVQLVQSGAEVKKPGASVKVSCKASGYTFTSHTMHWVRQAPGQRLEWIGYINPSSGYTNYNQKFKDKATLTADTSASTAYMELSSLRSEDTAVYYCARWDYVALDYWGQGTLVTVSS

SEQ ID NO: 40 - DNA, 443 bp, Artificial Sequence
  Description of Artificial Sequence Synthetic polynucleotide
actagtaccaccatggaaaggcactggatctttctcttcctgttgtcagtgactgcaggtgtccactcccaggtccagcttgtgcagtctggggctgaggtgaagaagcctggggcctcagtgaaggtttcctgcaaggcttctggatacaccttcactagccacaccatgcactgggtgcgccaggcccccggacaaaggcttgagtggattggatacattaatcctagcagtggttatactaattacaatcagaagttcaaggacaaggccacccttaccgccgacacatccgccagcacagcctacatggagctgagcagcctgagatctgaagacaccgctgtgtattactgtgccaggtgggattacgttgctctggactactggggccagggaaccctggtcaccgtctcctcaggtgagtctgtgtactgaagctt

SEQ ID NO: 41 - protein, 136 aa, Artificial Sequence
  Description of Artificial Sequence Synthetic polypeptide
MERHWIFLFLLSVTAGVHSQVQLVQSGAEVKKPGASVKVSCKASGYTFTSHTMHWVRQAPGQRLEWIGYINPSSGYTNYNQKFKDKATLTADTSASTAYMELSSLRSEDTAVYYCARWDYVALDYWGQGTLVTVSS

SEQ ID NO: 42 - protein, 5 aa, Artificial Sequence
  Description of Artificial Sequence Synthetic peptide
SHTMH

SEQ ID NO: 43 - protein, 17 aa, Artificial Sequence
  Description of Artificial Sequence Synthetic peptide
YINPSSGYTNYNQKFKD

SEQ ID NO: 44 - protein, 8 aa, Artificial Sequence
  Description of Artificial Sequence Synthetic peptide
WDYVALDY

SEQ ID NO: 45 - protein, 117 aa, Artificial Sequence
  Description of Artificial Sequence Synthetic polypeptide
QVQLVQSGAEVKKPGASVKVSCKASGYTFTSHTMHWVRQAPGQRLEWIGYINPSSGYTNYNQKFKDRATLTADTSASTAYMELSSLRSEDTAVYYCARWDYVALDYWGQGTLVTVSS

SEQ ID NO: 46 - DNA, 443 bp, Artificial Sequence
  Description of Artificial Sequence Synthetic polynucleotide
actagtaccaccatggaaaggcactggatctttctcttcctgttgtcagtgactgcaggtgtccactcccaggtccagcttgtgcagtctggggctgaggtgaagaagcctggggcctcagtgaaggtttcctgcaaggcttctggatacaccttcactagccacaccatgcactgggtgcgccaggcccccggacaaaggcttgagtggattggatacattaatcctagcagtggttatactaattacaatcagaagttcaaggacagagccacccttaccgccgacacatccgccagcacagcctacatggagctgagcagcctgagatctgaagacaccgctgtgtattactgtgccaggtgggattacgttgctctggactactggggccagggaaccctggtcaccgtctcctcaggtgagtctgtgtactgaagctt

SEQ ID NO: 47 - protein, 136 aa, Artificial Sequence
  Description of Artificial Sequence Synthetic polypeptide
MERHWIFLFLLSVTAGVHSQVQLVQSGAEVKKPGASVKVSCKASGYTFTSHTMHWVRQAPGQRLEWIGYINPSSGYTNYNQKFKDRATLTADTSASTAYMELSSLRSEDTAVYYCARWDYVALDYWGQGTLVTVSS

SEQ ID NO: 48 - protein, 11 aa, Artificial Sequence
  Description of Artificial Sequence Synthetic peptide
SASQGISNYLN

SEQ ID NO: 49 - protein, 7 aa, Artificial Sequence
  Description of Artificial Sequence Synthetic peptide
YISSLHS

SEQ ID NO: 50 - protein, 9 aa, Artificial Sequence
  Description of Artificial Sequence Synthetic peptide
QQYSKLPYT

SEQ ID NO: 51 - protein, 107 aa, Artificial Sequence
  Description of Artificial Sequence Synthetic polypeptide
DIQMTQSPSSLSASVGDRVTITCSASQGISNYLNWYQQKPGKVPKLLIYYISSLHSGVPSRFSGSGSGTDYTLTISSLQPEDVATYYCQQYSKLPYTFGGGTKVEIK

SEQ ID NO: 52 - DNA, 415 bp, Artificial Sequence
  Description of Artificial Sequence Synthetic polynucleotide
gctagcaccaccatgtcctctgctcagttccttggtctcctgttgctctgttttcaaggaaccagatgtgacatccagatgacccagtctccatcctccctgtctgcatctgtgggagacagagtcaccatcacttgcagtgcaagtcagggcattagcaattatctgaactggtatcagcagaaaccagggaaagttcctaagctcctgatctattacatctcaagtctgcactcaggggtcccatctcggttcagtggcagtggatctgggacagattatactctcaccatcagcagcctgcagcctgaagatgttgcaacttattactgtcaacagtatagtaagctgccgtacaccttcggcggagggaccaaagtggagatcaaacgtaagtgcactttcctaagaattc

SEQ ID NO: 53 - protein, 126 aa, Artificial Sequence
  Description of Artificial Sequence Synthetic polypeptide
MSSAQFLGLLLLCFQGTRCDIQMTQSPSSLSASVGDRVTITCSASQGISNYLNWYQQKPGKVPKLLIYYISSLHSGVPSRFSGSGSGTDYTLTISSLQPEDVATYYCQQYSKLPYTFGGGTKVEIK

SEQ ID NO: 54 - protein, 5 aa, Artificial Sequence
  Description of Artificial Sequence Synthetic peptide
DYNMD

SEQ ID NO: 55 - protein, 17 aa, Artificial Sequence
  Description of Artificial Sequence Synthetic peptide
DINPKYDSTTYNQKFKG

SEQ ID NO: 56 - protein, 16 aa, Artificial Sequence
  Description of Artificial Sequence Synthetic peptide
ERGSYDYDGTPYAMDY

SEQ ID NO: 57 - protein, 125 aa, Artificial Sequence
  Description of Artificial Sequence Synthetic polypeptide
QVQLVQSGAEVKKPGASVKVSCKASGYTFTDYNMDWVRQAPGQRLEWIGDINPKYDSTTYNQKFKGKATLTADTSASTAYMELSSLRSEDTAVYYCARERGSYDYDGTPYAMDYWGQGTLVTVSS

SEQ ID NO: 58 - DNA, 468 bp, Artificial Sequence
  Description of Artificial Sequence Synthetic polynucleotide
actagtaccaccatggaatggagctggatctttctctttctcctgtcaggaactgcaggcgtcctctctcaggtccagcttgtgcagtctggggctgaagtgaagaagcctggggcctcagtgaaagtttcctgcaaggcttctggatacaccttcactgactacaacatggactgggtgcgccaggcccccggacaaaggcttgagtggattggagatattaatcctaagtatgatagtactacctacaaccagaagttcaagggaaaggccacccttaccgcagacacatccgccagcacagcctacatggagctcagcagcctgagatctgaagacaccgctgtgtattactgtgccagggagaggggctcctatgattacgacgggactccctatgctatggactactggggccagggaaccctggtcaccgtctcctcaggtgagtctgctgtactgaagctt

SEQ ID NO: 59 - protein, 144 aa, Artificial Sequence
  Description of Artificial Sequence Synthetic polypeptide
MEWSWIFLFLLSGTAGVLSQVQLVQSGAEVKKPGASVKVSCKASGYTFTDYNMDWVRQAPGQRLEWIGDINPKYDSTTYNQKFKGKATLTADTSASTAYMELSSLRSEDTAVYYCARERGSYDYDGTPYAMDYWGQGTLVTVSS

SEQ ID NO: 60 - protein, 5 aa, Artificial Sequence
  Description of Artificial Sequence Synthetic peptide
DYNMD

SEQ ID NO: 61 - protein, 17 aa, Artificial Sequence
  Description of Artificial Sequence Synthetic peptide
DINPKYDSTTYNQKFKG

SEQ ID NO: 62 - protein, 16 aa, Artificial Sequence
  Description of Artificial Sequence Synthetic peptide
ERGSYDYDGTPYAMDY

SEQ ID NO: 63 - protein, 125 aa, Artificial Sequence
  Description of Artificial Sequence Synthetic polypeptide
QVQLVQSGAEVKKPGASVKVSCKASGYTFTDYNMDWVRQAPGQRLEWIGDINPKYDSTTYNQKFKGRATLTADTSASTAYMELSSLRSEDTAVYYCARERGSYDYDGTPYAMDYWGQGTLVTVSS

SEQ ID NO: 64 - DNA, 468 bp, Artificial Sequence
  Description of Artificial Sequence Synthetic polynucleotide
actagtaccaccatggaatggagctggatctttctctttctcctgtcaggaactgcaggcgtcctctctcaggtccagcttgtgcagtctggggctgaagtgaagaagcctggggcctcagtgaaagtttcctgcaaggcttctggatacaccttcactgactacaacatggactgggtgcgccaggcccccggacaaaggcttgagtggattggagatattaatcctaagtatgatagtactacctacaaccagaagttcaagggaagagccacccttaccgcagacacatccgccagcacagcctacatggagctcagcagcctgagatctgaagacaccgctgtgtattactgtgccagggagaggggctcctatgattacgacgggactccctatgctatggactactggggccagggaaccctggtcaccgtctcctcaggtgagtctgctgtactgaagctt

SEQ ID NO: 65 - protein, 144 aa, Artificial Sequence
  Description of Artificial Sequence Synthetic polypeptide
MEWSWIFLFLLSGTAGVLSQVQLVQSGAEVKKPGASVKVSCKASGYTFTDYNMDWVRQAPGQRLEWIGDINPKYDSTTYNQKFKGRATLTADTSASTAYMELSSLRSEDTAVYYCARERGSYDYDGTPYAMDYWGQGTLVTVSS

SEQ ID NO: 66 - protein, 17 aa, Artificial Sequence
  Description of Artificial Sequence Synthetic peptide
KSSQSLLYSSNRKNYLA

SEQ ID NO: 67 - protein, 7 aa, Artificial Sequence
  Description of Artificial Sequence Synthetic peptide
WASTRES

SEQ ID NO: 68 - protein, 9 aa, Artificial Sequence
  Description of Artificial Sequence Synthetic peptide
QQYYTYPRT

SEQ ID NO: 69 - protein, 113 aa, Artificial Sequence
  Description of Artificial Sequence Synthetic polypeptide
DIVMTQSPDSLAVSLGERATINCKSSQSLLYSSNRKNYLAWYQQKPGQPPKLLIYWASTRESGVPDRFSGSGSGTDFTLTISSLQAEDVAVYYCQQYYTYPRTFGQGTKVEIK

SEQ ID NO: 70 - DNA, 435 bp, Artificial Sequence
  Description of Artificial Sequence Synthetic polynucleotide
gctagcaccaccatggattcacaggcccaggttcttatgttgctgctgctctgggtgtctgggacctgtggggacatcgtgatgacccagtctccagactccctggctgtgtctctgggcgagagggccaccatcaactgcaagtccagccagagtcttctgtacagctccaaccgcaagaactacctggcttggtaccagcagaaaccaggacagcctcctaagttgctcatttactgggcatctacccgggaatccggggtccctgaccgattcagtggcagcgggtctgggacagatttcactctcaccatcagcagcctgcaggctgaagatgtggcagtttattactgtcagcaatattatacctatcctcggaccttcggccaagggaccaaagtggaaatcaaacgtgagtagaatttaaacgaattc

SEQ ID NO: 71 - protein, 133 aa, Artificial Sequence
  Description of Artificial Sequence Synthetic polypeptide
MDSQAQVLMLLLLWVSGTCGDIVMTQSPDSLAVSLGERATINCKSSQSLLYSSNRKNYLAWYQQKPGQPPKLLIYWASTRESGVPDRFSGSGSGTDFTLTISSLQAEDVAVYYCQQYYTYPRTFGQGTKVEIK

SEQ ID NO: 72 - protein, 117 aa, Mus sp.
QVQLQQSGAELARPGASVKMSCKASGYTFTSHTMHWVKQRPGQGLEWIGYINPSSGYTNYNQKFKDKATLTADKSSSTAYMQLSSLTSEDSAVYYCARWDYVALDYWGQGTSVTVSS

SEQ ID NO: 73 - protein, 107 aa, Mus sp.
EIQMTQTTSSLSASLGDRVTISCSASQGISNYLNWYQQKPDGTVKLLIYYISSLHSGVTSRFSGSGSGTDYSLTISNLEPEDIATYYCQQYSKLPYTFGGGTKLEIK

SEQ ID NO: 74 - protein, 125 aa, Mus sp.
EVQLQQFGAELVKPGASVKISCKASGYTFTDYNMDWVKQSHGKSLEWIGDINPKYDSTTYNQKFKGKATLTADKSSSTAYMELRSLTSEDTAVYYCARERGSYDYDGTPYAMDYWGQGTSVTVSS

SEQ ID NO: 75 - protein, 113 aa, Mus sp.
DIVMSQSPSSLAVSVGEKVTMSCKSSQSLLYSSNRKNYLAWYQQKPGQSPKLLIYWASTRESGVPDRFTGSGSGTDFTLTISSVKAEDLAVYYCQQYYTYPRTFGGGTKLEIK

SEQ ID NO: 76 - protein, 116 aa, Mus sp.
QVQLQQPGAELVKPGASVKLSCKASGYTFTSYWVNWVKQRPGQGLEWIGEINPSNGRTDYNEKFKSKAIMIVDKSSSTAYMQFSSLTSEDSAVYYCAGGLDYFDYWGQGTTLTVSS

SEQ ID NO: 77 - protein, 107 aa, Mus sp.
NIVMTQSPKSMSMSVGERVTLSCKASENVGTYVSWYQQKPDQSPKLLIYGASNRYTGVPDRFTGSGSATDFTLTISSVQAEDLADYHCGQSYSYPFTFGSGTSLEIK